(19) United States
(12) Patent Application Publication
(10) Pub. No.: US 2026/0000662 A1
(43) Pub. Date: Jan. 1, 2026

(54) USE OF ARIMOCLOMOL IN ACTIVATING CLEAR GENE EXPRESSION AS TREATMENT FOR LYSOSOMAL STORAGE DISORDERS

(71) Applicant: Zevra Therapeutics, Inc., Celebration, FL (US)

(72) Inventors: Sven Guenther, Coralville, IA (US); Travis Mickle, Kissimmee, FL (US); Thomas Kirkegaard Jensen, Rødovre (DK)

(21) Appl. No.: 19/255,733

(22) Filed: Jun. 30, 2025

Related U.S. Application Data
(60) Provisional application No. 63/666,647, filed on Jul. 1, 2024.
(60) Provisional application No. 63/666,650, filed on Jul. 1, 2024.
(60) Provisional application No. 63/676,313, filed on Jul. 26, 2024.
(60) Provisional application No. 63/684,069, filed on Aug. 16, 2024.
(60) Provisional application No. 63/697,270, filed on Sep. 20, 2024.
(60) Provisional application No. 63/746,677, filed on Jan. 17, 2025.
(60) Provisional application No. 63/815,174, filed on May 30, 2025.
(60) Provisional application No. 63/822,129, filed on Jun. 11, 2025.

(51) Int. Cl. A61K 31/4545; A61P 3/00

(52) U.S. Cl. CPC A61K 31/4545; A61P 3/00

(57) ABSTRACT

The present disclosure provides methods of treating lysosomal storage disorders such as Niemann-Pick Disease, Type-C, by administering a therapeutically effective amount of arimoclomol. The present disclosure further provides methods of removing lysosomal cholesterol by administering the same.

### STATEMENT OF RELATED APPLICATION

This application claims priority to U.S. Application No. 63/822,129, filed on Jun. 11, 2025, U.S. Application No. 63/815,174, filed on May 30, 2025, U.S. Application No. 63/746,677, filed on Jan. 17, 2025, U.S. Application No. 63/697,270, filed Sep. 20, 2024, U.S. Application No. 63/684,069, filed Aug. 16, 2024, U.S. Application No. 63/676,313, filed Jul. 26, 2024, and U.S. Application No. 63/666,650, filed on Jul. 1, 2024, all of which are incorporated by reference in their entirety herein.

### INTRODUCTION

Neimann-Pick disease, type C (NPC) is an ultra-rare, severely debilitating neurodegenerative lysosomal storage disease whose hallmark clinical complication is progressive encephalopathy via neuronal death that culminates with patients unable to move or communicate in a severe and universally fatal dementia. Although NPC can manifest at any age, it is usually diagnosed in early childhood, and the estimated median age of death is 13 years (Bianconi et al., 2019). There are no FDA-approved disease-modifying therapies for NPC, a disease that uniformly places a tremendous burden on patients, families, and caregivers. The incidence of NPC is approximately 1:100,000 live births in the United States (US) (Geberhiwot et al., 2018), where it affects an estimated 600-900 patients (Burton et al., 2021).

NPC is caused by mutations in the NPC1 (˜95% of cases) or NPC2 (˜5% of cases) genes. Patients with a double functional null mutation have the most rapid disease progression. The consequence of diminished function of either gene is lysosomal intracellular transport dysfunction, resulting in accumulation of cholesterol in lysosomes and therefore a myriad of negative downstream consequences, including cell stress, toxicity, and death. Organs most affected by a buildup of cholesterol include the liver, spleen, and brain, with neurodegeneration of the brain being the main driver of clinical manifestations of NPC.

Despite decades of research and development, there are no approved disease-modifying therapies for patients with NPC in the US, and there are no biomarkers correlated with severity or survival. Patients experience a variety of heterogeneous and disabling neurological signs and symptoms that can require frequent hospitalizations (Imrie et al., 2007; Vanier, 2010; Patterson et al., 2012). Earlier onset of neurological signs and symptoms is predictive of more rapid disease progression (Vanier 2010). At the terminal stage, patients cannot leave their beds due to severe encephalopathy and uncontrolled seizures.

Miglustat is authorized in the European Union (EU) for the treatment of progressive neurological manifestations in patients with NPC (EMA EPAR Zavesca®) and is approved in the US for the treatment of another lysosomal storage disease, mild/moderate type 1 Gaucher's disease (GD) (Zavesca® USPI). While considered the standard of care globally for NPC, miglustat is only available off-label in the US. Current treatment relies largely on symptomatic management including anti-epileptics, anti-spastics, and baclofen, and care from a multidisciplinary team.

With its profoundly negative impact on a patient's daily function, quality of life, and heavy burden on caregivers, there is an urgent unmet medical need for effective and safe pharmacological interventions. The impact on patients with NPC and their caregivers cannot be overstated, given the severe, rapid, and relentless nature of the disease.

Recently, it has been reported that the NPC may be treated by administering a cyclodextrin, which increases the expression of Transcription Factor EB (TFEB). [Singhal et al. Nature Scientific Reports, 2020, 10:8663]. may lead to increased expression of Coordinated Lysosomal Expression and Regulation (CLEAR) network proteins. Notably, cyclodextrin does not appear to change the ratio of cytosolic and nuclear TFEB.

There remains a need in the art for a combination therapy that combines an orally available small molecule method with miglustat for treating NPC that targets the etiology of NPC.

Without wanting to be limited by any one theory, it is believed that cellular levels of proteins encoded by CLEAR genes including the NPC1 gene can be increased by activating TFEB and/or TFE3. It is also believed that there are two pools of these transcription factors-one inside the cytosol and one inside the cell nucleus—and that the nuclear forms represent the activated form. Therefore, increasing the levels of TFEB and/or TFE3 in the nucleus promotes their activation. The nuclear levels of TFEB and/or TFE3 can be increased by either overexpressing these transcription factors without significantly affecting their cytosolic and nuclear ratio, or by shifting the ratio from their cytosolic form to the nuclear forms without increasing their expression levels, or a combination of both.

Arimoclomol is an orally bioavailable small molecule. Arimoclomol oral formulations were developed to support administration that can be swallowed whole or the contents of which can be emptied into liquids or soft foods. Arimoclomol has a novel mechanism of action (MOA), which targets the fundamentals of NPC etiology by increasing the translocation of TFEB and/or TFE3 from the cytosol into the cell nucleus. As a result, arimoclomol upregulates genes of the CLEAR network including NPC1. The overexpression of the NPC1 gene increases the biosynthesis of NPC1 protein.

### BRIEF SUMMARY OF THE INVENTION

The present disclosure provides methods for increasing the removal of lysosomal cholesterol from cells of a patient in need thereof. The present disclosure further provides methods of removing endosomal cholesterol from cells of a patient in need thereof. The present disclosure still further provides methods for increasing cellular production of the protein NPC1 in a patient in need thereof. The present disclosure additionally provides methods of improving the score of at least one domain of the 4-domain Neimann Pick Type C Clinical Severity Scale (4D-NPCCSS) in a patient in need thereof. The present disclosure still further provides methods of reducing and/or delaying the progression of Nieman-Pick disease, Type C, in a patient in need thereof.

In one aspect, the present disclosure related to a method of increasing removal of lysosomal cholesterol from cells of a patient in need thereof, the method comprising the step of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

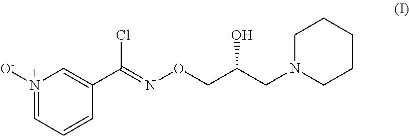

or a pharmaceutically acceptable salt thereof, where increasing the activation of the at least one transcription factor increases the removal of lysosomal cholesterol from the cells of the patient relative to a patient not administered a therapeutically effective amount of the compound of Formula I, or a pharmaceutically acceptable salt thereof.

In yet another aspect, the present technology relates to a method of increasing removal of lysosomal cholesterol from cells of a patient/subject with moderate to severe renal impairment, the method comprising the steps of: determining the Estimated Glomerular Filtration Rate (eGFR) of the subject/patient; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

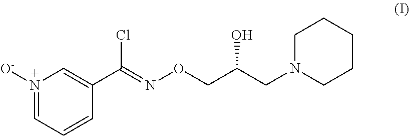

or a pharmaceutically acceptable salt thereof, where increasing the activation of the at least one transcription factor increases the removal of lysosomal cholesterol from the cells of the patient relative to a patient not being administered a therapeutically effective amount of the compound having a structure of Formula I, or a pharmaceutically acceptable salt thereof.

In one aspect, the present technology relates to a method of increasing removal of endosomal cholesterol from cells of a patient in need thereof, the method comprising the step of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

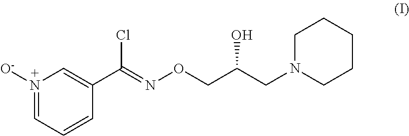

or a pharmaceutically acceptable salt thereof, where increasing the activation of the at least one transcription factor increases the removal of endosomal cholesterol from the cells of the patient relative to a patient not being administered a therapeutically effective amount of the compound having a structure of Formula I, or a pharmaceutically acceptable salt thereof.

In yet another aspect, the present technology relates to a method of removing endosomal cholesterol from cells of a patient/subject with moderate to severe renal impairment, the method comprising the steps of: determining the Estimated Glomerular Filtration Rate (eGFR) of the subject/patient; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

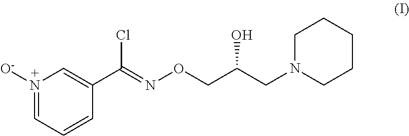

or a pharmaceutically acceptable salt thereof, where increasing the activation of the at least one transcription factor increases the removal of endosomal cholesterol from the cells of the patient relative to a patient not being administered a therapeutically effective amount of the compound having a structure of Formula I, or a pharmaceutically acceptable salt thereof.

In one aspect, the present technology related to a method of increasing cellular production of NPC1 protein in a patient with a mutant Niemann-Pick type-C Protein 1 (NPC1) with decreased functionality compared to wild-type NPC1 protein, the method comprising the step of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

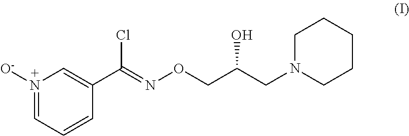

or a pharmaceutically acceptable salt thereof, where increasing the activation of the at least one transcription factor increases cellular production of the mutant NPC1 protein having reduced functionality compared to wild-type NPC1 protein relative to a patient not being administered a therapeutically effective amount of the compound having a structure of Formula I, or a pharmaceutically acceptable salt thereof.

In yet another aspect, the present technology relates to a method of increasing cellular production of NPC1 protein in a patient with a mutant Niemann-Pick type-C Protein 1 (NPC1) with decreased functionality, and moderate to severe renal impairment, the method comprising the steps of: determining the Estimated Glomerular Filtration Rate (eGFR) of the patient with a mutant Niemann-Pick type-C Protein 1 (NPC1) with decreased functionality; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

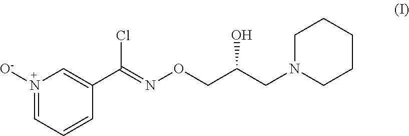

or a pharmaceutically acceptable salt thereof; where increasing the activation of the at least one transcription factor increases cellular production of the mutant NPC1 protein having reduced functionality relative to a patient not being administered a therapeutically effective amount of the compound having a structure of Formula I, or a pharmaceutically acceptable salt thereof.

In one aspect, the present technology relates to a method for improving at least one 4D-NPCCSS scale domain score in a patient in need thereof, the method comprising the step of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

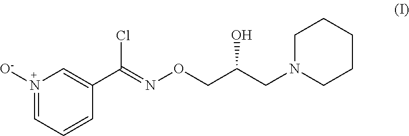

or a pharmaceutically acceptable salt thereof, where increasing the activation of the at least one transcription factor decreases at least one 4D-NPCCSS domain score, wherein the at least one 4D-NPCCSS domain is selected from ambulation, fine motor skills, and speech relative to a patient not being administered a therapeutically effective amount of the compound having a structure of Formula I, or a pharmaceutically acceptable salt thereof.

In yet another aspect, the present technology relates to a method for improving at least one 4D-NPCCSS scale domain score in a patient in need thereof with moderate to severe renal impairment, the method comprising the steps of: determining the Estimated Glomerular Filtration Rate (eGFR) of the patient with a mutant Niemann-Pick type-C Protein 1 (NPC1) with decreased functionality; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

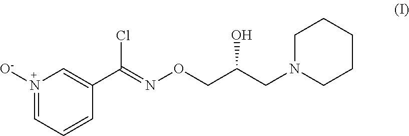

or a pharmaceutically acceptable salt thereof, where increasing the activation of the at least one transcription factor decreases at least one 4D-NPCCSS domain score, wherein the at least one 4D-NPCCSS domain is selected from ambulation, fine motor skills, and speech relative to a patient not being administered a therapeutically effective amount of the compound having a structure of Formula I, or a pharmaceutically acceptable salt thereof.

In one aspect, the present technology relates to a method for reducing NPC disease progression in a patient in need thereof, the method comprising the step of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

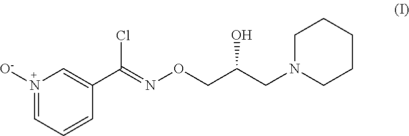

or a pharmaceutically acceptable salt thereof, wherein administering the compound having a structure of Formula I reduces the progression of NPC disease as compared to a placebo group.

In yet another aspect, the present technology relates to a method for reducing NPC disease progression in a patient in need thereof with moderate to severe renal impairment, the method comprising the steps of: determining the Estimated Glomerular Filtration Rate (eGFR) of the patient with a mutant Niemann-Pick type-C Protein 1 (NPC1) with decreased functionality; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

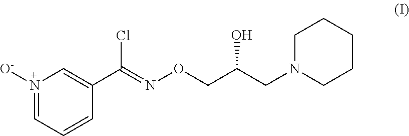

or a pharmaceutically acceptable salt thereof, wherein administering the compound having a structure of Formula I reduces the progression of NPC disease as compared to a placebo group.

In one aspect, the present technology relates to a method of delaying NPC disease progression in a patient in need thereof, the method comprising the step of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

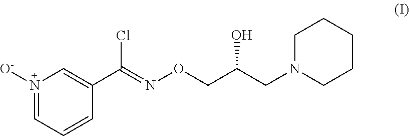

or a pharmaceutically acceptable salt thereof, wherein administering the compound having a structure of Formula I delays the progression of NPC disease as compared to a placebo group.

In yet another aspect, the present technology relates to a method of delaying NPC disease progression in a patient in need thereof, with moderate to severe renal impairment, the method comprising the steps of: determining the Estimated Glomerular Filtration Rate (eGFR) of the patient with a mutant Niemann-Pick type-C Protein 1 (NPC1) with decreased functionality; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

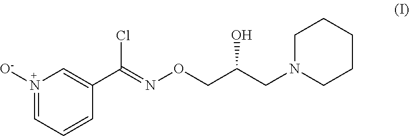

or a pharmaceutically acceptable salt thereof, wherein administering the compound having a structure of Formula I reduces the progression of NPC disease as compared to a placebo group.

### BRIEF SUMMARY OF THE FIGURES

FIG. 1 shows the role of NPC1 in Normal Lysosomal Function.

FIG. 2 shows the role of NPC1 in untreated NPC disease state.

FIG. 3 shows the mechanism of action of arimoclomol in the NPC disease state.

FIG. 4 shows the mechanism of action of combined arimoclomol and miglustat treatment improving lysosomal function.

FIG. 5 shows the mechanism of action through which arimoclomol activates TFEB and/or TFE3 thereby upregulating CLEAR gene expression of NPC1 protein.

FIG. 6 shows the TFE3 nucleus-cytosol Filipin staining intensity ratios in healthy human (WT) and human NPC fibroblasts.

FIG. 7 shows the TFE3 and/or TFEB nucleus-cytosol intensity ratios in healthy human (WT) fibroblasts and HeLa cells treated with arimoclomol in combination with NPC1 inhibitor U-18666A.

FIG. 8 shows the effect of arimoclomol on the expression of TFEB and TFE3.

FIG. 9 shows the enhanced binding of TFE3 to CLEAR elements in target gene promoters after arimoclomol treatment.

FIG. 10 shows the expression levels of selected CLEAR network genes after treatment with 400 μM Arimoclomol in wild type fibroblasts.

FIG. 11 shows the expression levels of selected CLEAR network genes and HSPA1A after treatment with arimoclomol and miglustat.

FIG. 12 shows the effect of arimoclomol on gene expression of NPC1, GBA, and HSPA1A.

FIG. 13 shows the NPC1 protein concentrations in NPC patient fibroblast cell lines relative to wild type.

FIG. 14 shows the effects of arimoclomol treatment on NPC1 protein concentrations in NPC fibroblasts with different mutations.

FIG. 15 shows the mean increase in NPC1 protein level expression in NPC patient fibroblasts as a function of increasing concentration of arimoclomol.

FIG. 16 shows the results of Endo H assays with arimoclomol to assess NPC1 protein maturation and localization.

FIG. 17 shows the quantification of unesterified cholesterol levels in the lysosomal compartment of human NPC fibroblasts after arimoclomol treatment as measured by Filipin staining intensity.

FIG. 18 shows the survival beyond 75, 80, and 85 Days in female Npc1^{-/-} mice treated with arimoclomol (30 mg/kg/day) or vehicle control.

FIG. 19 shows the concentration of mature isoform 1 NPC1 protein in the brains of NPC-dependent mice.

FIG. 20 shows the Filipin staining intensity vs control in lysosomal compartments of NPC (I1061T/I1061T) human fibroblasts following treatment with arimoclomol and miglustat for 7 and 14 days.

FIG. 21 shows the survival beyond 16, 17, and 18 weeks of age in female Npc^{1nmf164} mutant mice treated with arimoclomol.

FIG. 22 shows the decrease in unesterified cholesterol determined by Filipin Staining based on exposure to increasing arimoclomol concentrations.

FIG. 23 shows the survival beyond 75, 80, and 85 days of age in female Npc1^{-/-} mutant mice treated with arimoclomol plus miglustat or vehicle control.

FIG. 24 shows the survival of Npc1^{-/-} mice treated with a combination of arimoclomol and miglustat vs vehicle control.

FIG. 25 shows the number of rearing events in wild type mice, untreated, and arimoclomol or arimoclomol/miglustat treated NPC mutant mice.

FIG. 26 shows the change in total rearing events in NPC-independent and NPC-dependent mice with arimoclomol treatment.

FIG. 27 shows the change in survival time in NPC-independent and NPC-dependent mice with arimoclomol treatment.

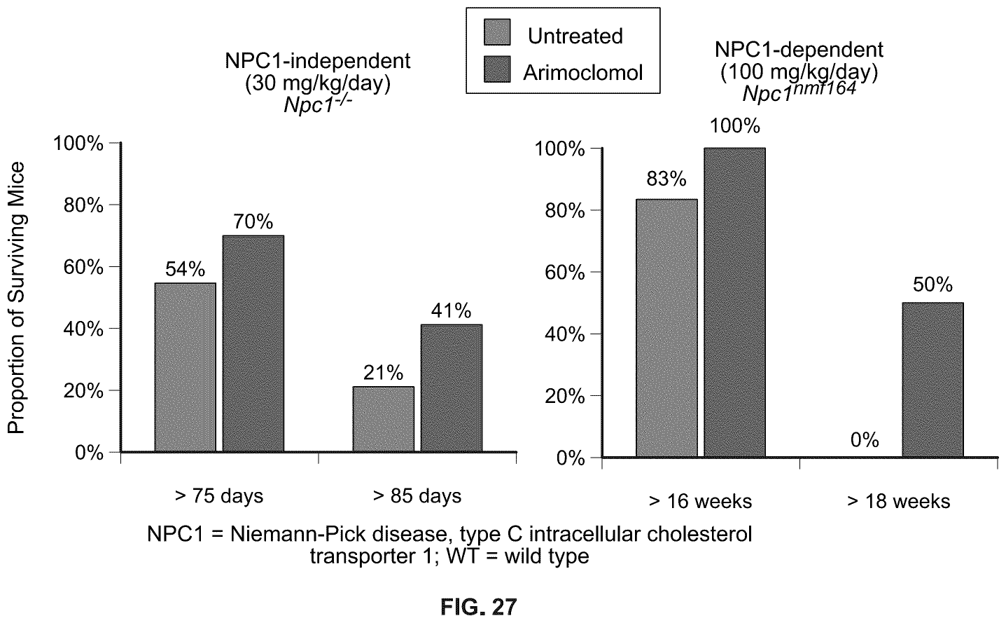

FIG. 28 shows the total cholesterol levels in the liver of wild type, untreated, and arimoclomol-treated Npc^{nmf164} mice.

FIG. 29 shows the concentrations of NPC1 protein in the liver of wild type, untreated and arimoclomol-treated Npc^{nmf164} mice.

FIG. 30 shows the myelin basic protein levels in the brain of wild type, untreated, and arimoclomol-treated Npc^{nmf16}4 mice.

FIG. 31 shows the results of double-blind studies measuring the change from baseline in 4D-NPCCSS for patients administered arimoclomol (top) and arimoclomol and miglustat (bottom) compared to placebo.

### DETAILED DESCRIPTION

The present disclosure provides methods of treating Niemann-Pick Disease, Type C, in a patient in need thereof by administering to the patient in need thereof a composition comprising a therapeutically effective amount of arimoclomol or a pharmaceutically acceptable salt thereof. The present disclosure still further provides methods of increasing removal of lysosomal cholesterol from the cells of a patient in need thereof suffering from a lysosomal storage disorder. The present disclosure additionally provides methods of removing endosomal cholesterol from the cells of a patient in need thereof suffering from a lysosomal storage disorder. The present disclosure further provides methods of increasing cellular expression of the NPC1 protein in a patient suffering from a lysosomal storage disorder. Finally, the present disclosure provides methods for improving at least one 4-D NPCCSS score in a patient suffering from a lysosomal storage disorder.

### I. INTRODUCTION

NPC is a genetic disease, inherited in an autosomal recessive pattern, where both parents contribute a mutated NPC1 or NPC2 gene to their child. In 95% of cases, NPC is caused by mutations in the NPC1 gene (Vanier, 2010; Geberhiwot et al., 2018). Double functional null NPC1 genotype predicts an early infantile age of onset and severe NPC. NPC1 and NPC2 genes encode lysosomal proteins that are essential in intracellular transport and metabolism of cholesterol and other lipids. The dysfunction of either of these NPC genes results in a reduced amount of properly folded and mature NPC1 protein. The consequence is lysosomal dysfunction with accumulation of lipids resulting in a negative impact on downstream pathways. For example, lysosomal sphingolipid degradation becomes impaired leading to accumulation of several complex sphingolipids in various tissues including the brain and liver (Vanier and Latour, 2015; Breiden and Sandhoff, 2020). This lipid accumulation is cytotoxic and causes neurodegeneration and peripheral organ dysfunction (Lloyd-Evans and Platt, 2010; Platt, 2018). Visceral, systemic forms of the disease present first, including cholestasis and an enlarged spleen. NPC is characterized by a variety of heterogenous disabling symptoms including epilepsy and difficulties with basic functions such as walking, motor coordination, swallowing, speaking, concentrating, and remembering, that can require frequent hospitalization (Vanier, 2010; Stampfer et al., 2013; Wraith et al., 2014; Patterson et al., 2017).

NPC is a very heterogeneous, relentlessly progressive, neurodegenerative disease that ultimately results in early death. Non-specific symptoms present in irregular patterns that vary by person—with some progressing rapidly in a 12-month period and some progressing more slowly. Patients endure a progressive and substantial decline in both function and quality of life. This impact on patients and their caregivers cannot be understated given the nature of the disease, the burden of care, and the emotional toll on caregivers as the patients afflicted with the most severe and progressive manifestations of the disease are often young children.

As no FDA-approved pharmaceutical therapies are available for the treatment of NPC, there is a high unmet medical need for effective and safe pharmacological treatments that can delay the progression of this severely debilitating and fatal disease.

The inventors unexpectedly discovered that oral administration of arimoclomol activates the Coordinated Lysosomal Expression and Regulation (“CLEAR”) network of genes by translocating Transcription Factor EB (TFEB) and Transcription Factor E3 (TFE3) from the cytosol to the cell nucleus without meaningfully changing their total cell concentrations. The resulting higher nucleus-to-cytosol concentration ratios of these transcription factors increase their binding to CLEAR network genes. The CLEAR genes contain the gene encoding the Niemann-Pick, Type C Proteins 1 and 2 (NPC1 and NPC2) which are instrumental in cholesterol trafficking. Increasing the expression of the NPC1 protein removes cholesterol from neuronal cells, which in turns slows the progression of the NPC disease.

Reference will now be made in detail to exemplary embodiments of the claimed invention. While the claimed invention will be described in conjunction with the exemplary embodiments, it will be understood that it is not intended to limit the claimed invention to those embodiments. To the contrary, it is intended to cover alternatives, modifications, and equivalents, as may be included within the spirit and scope of the claimed invention, as defined by the appended claims.

Those of ordinary skill in the art may make modifications and variations to the embodiments described herein without departing from the spirit or scope of the claimed invention. In addition, although certain methods and materials are described herein, other methods and materials that are similar or equivalent to those described herein can also be used to practice the claimed invention.

In addition, any of the compositions or methods provided, disclosed, or described herein can be combined with one or more of any of the other compositions and methods provided, disclosed, or described herein.

### Definitions

“A” and “an” as it relates to the present technology, means the singular form, but includes the plural form unless clear from the context.

“About” as it related to the present technology means, as it applied to measured quantities, +/−10% of the stated measured value; for example “about 100 mg” means 100 mg+/−10%, i.e. 90-110 mg. Unless specifically stated or obvious from context, as used herein, the term “about” is understood as within a range of normal tolerance in the art, for example, within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “or” means, and is used interchangeably with, the term “and/or,” unless context clearly indicates otherwise.

As used herein, the term “such as” means, and is used interchangeably with, the phrase “such as, for example” or “such as but not limited.”

As used herein, the term “subject” means a human or animal, including but not limited to a human or animal patient.

“Treating” as it related to the present technology means any of the following: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

“Isoform 1” as it relates to the present technology means a mature form of NPC1 protein.

“Maturation,” “maturing,” or “mature” as it relates to the present technology means the process and/or characteristic of a protein reaching its folded state.

“Mechanism of Action of Arimoclomol” or “Arimoclomol MOA” as it related to the present technology means the biological pathway by which arimoclomol treats the NPC Disease State. Arimoclomol targets NPC etiology by both NPC1-independent and NPC1-dependent pathways: (1) via the NPC1-independent pathway, arimoclomol upregulates expression of all CLEAR genes, thereby mitigating the deleterious effects of impaired cholesterol trafficking and improving overall cell health; (2) via the NPC1-dependent pathway, CLEAR gene upregulation increases production of the NPC1 protein. Though still mutated, overproducing the reduced function protein improves lysosomal function and cholesterol elimination as compared to normal level(s) of production of the reduced function protein (NPC1).

“Myelin Basic Protein” and/or “MBP” as it relates to the present technology means a protein believed to be important in the process of myelination of nerves in the nervous system.

The myelin sheath is a multi-layered membrane, unique to the nervous system, that functions as an insulator to greatly increase the velocity of axonal impulse conduction. MBP maintains the correct structure of myelin, interacting with the lipids in the myelin membrane.

“Rearing” as used herein, means a functional behavior in rodents that plays an important role in exploring and interacting with the environment. As such, rearing is a complex behavior that involves aspects such as locomotion, balance, exploratory drive, spatial awareness, cognitive mapping, sequence learning, and decision making. Therefore, the ability to rear is not simply an indicator of muscle function but a broader marker of brain health in rodents. The same brain regions (particularly the cerebellum, hippocampus, and midbrain) that are responsible for controlling rearing behavior in rodents are involved in recruiting muscles during movements related to fine motor function and swallow in humans. Rearing activity in NPC mice is therefore an appropriate indicator of neuronal health in brain regions relevant to functional endpoints like the Fine Motor Skills and Swallow domains of the NPCCSS in human NPC patients.

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

### II. COMPOUNDS OF FORMULA I AND PHARMACEUTICALLY ACCEPTABLE SALTS THEREOF

In an embodiment, the present disclosure provides arimoclomol or a pharmaceutically acceptable salt thereof. “Arimoclomol” as it related to the present technology means N-[2-hydroxy-3-(1-piperidinyl)-propoxy]-pyridine-1-oxide-3-carboximidoyl chloride, its stereoisomers and the acid addition salts thereof. Stereoisomers of arimoclomol include, but are not limited to (+)-(R)-N-[2-hydroxy-3-(1-piperidinyl)-propoxy]-pyridine-1-oxide-3-carboximidoyl chloride, (−)-(S)-N-[2-hydroxy-3-(1-piperidinyl)-propoxy]-pyridine-1-oxide-3-carboximidoyl chloride,(Z)-(R)-N-[2-hydroxy-3-(1-piperidinyl)-propoxy]-pyridine-1-oxide-3-carboximidoyl chloride,(E)-(R)-N-[2-hydroxy-3-(1-piperidinyl)-propoxy]-pyridine-1-oxide-3 carboximidoyl chloride,(Z)-(S)-N-[2-hydroxy-3-(1-piperidinyl)-propoxy]-pyridine-1-oxide-3-carboximidoyl chloride; and (E)-(S)-N-[2-hydroxy-3-(1-piperidinyl)-propoxy]-pyridine-1-oxide-3-carboximidoyl chloride. Arimoclomol has a structure of Formula I:

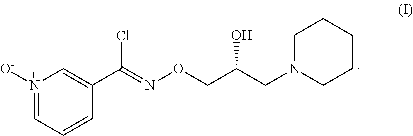

“Pharmaceutically acceptable salt” as it relates to the present technology means a salt which is not harmful to the subjects. Such salts include pharmaceutically acceptable basic or acid addition salts as well as pharmaceutically acceptable metal salts, ammonium salts and alkylated ammonium salts.

Acid addition salts include, but are not limited to, salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 66, 2, (1977) which is incorporated herein by reference.

Other pharmaceutically acceptable salts include, but are not limited to, acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, or undecylenate. In the preferred embodiments, the anionic salt form is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite.

In one alternative embodiment, the pharmaceutically acceptable salt of arimoclomol is arimoclomol citrate (N-[2-hydroxy-3-(1-piperidinyl)-propoxy]-pyridine-1-oxide-3-carboximidoyl chloride citrate).

An overview of the four-steps process for synthesizing and purifying arimoclomol and the pharmaceutically acceptable salts thereof is outlined below:

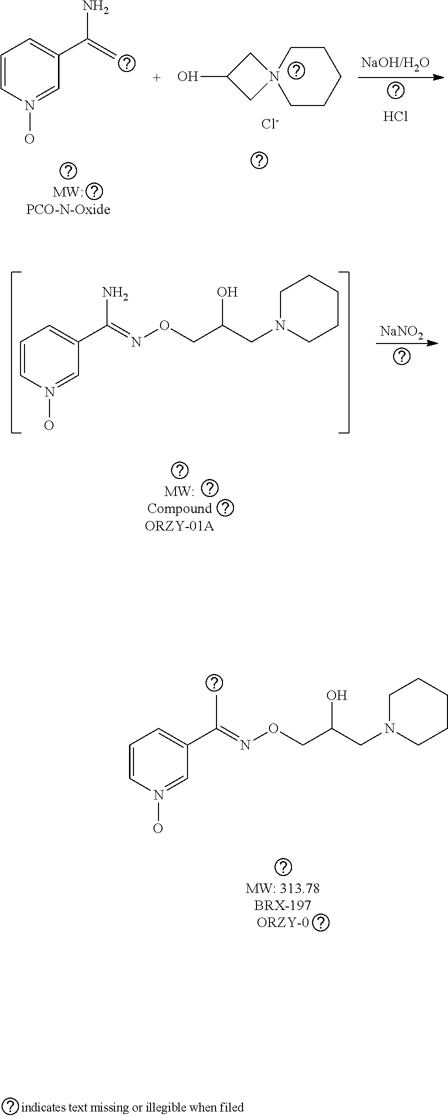

### Step 1: Overview of Process for Preparing ORZY-01

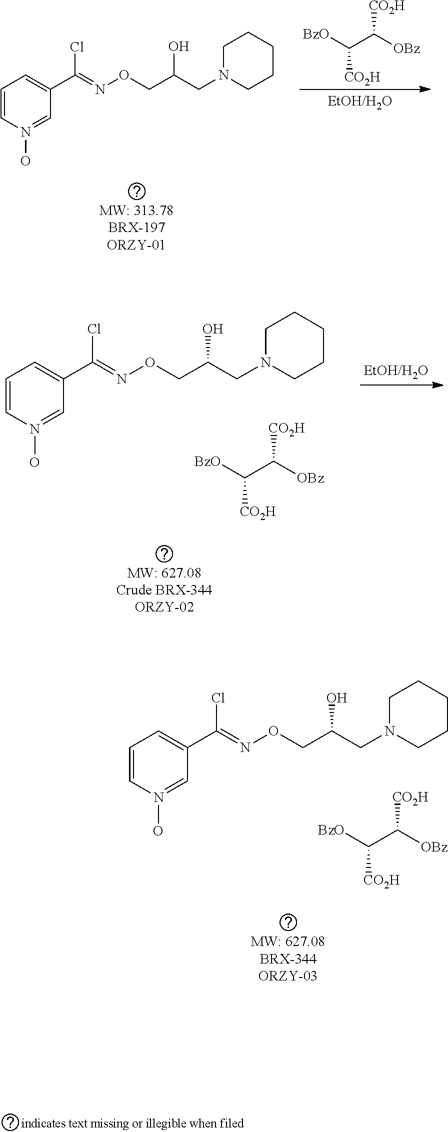

### Step 2: Overview of Process for Preparing ORZY-03

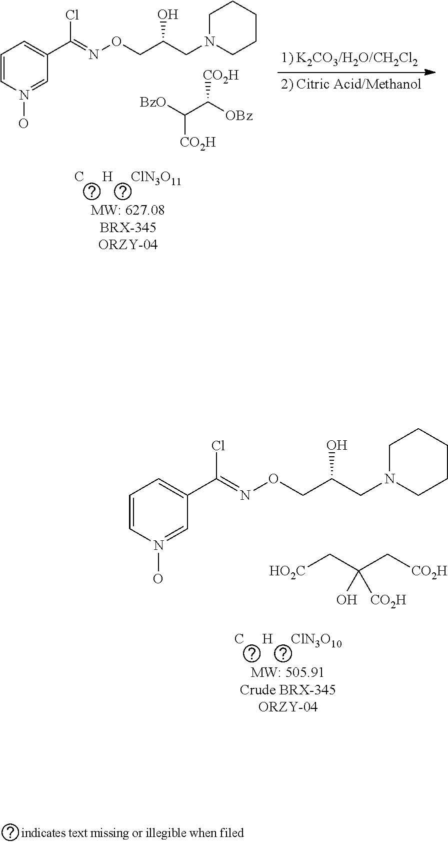

### Step 3: Overview of Process for Preparing Crude BRX-345 (ORZY-04)

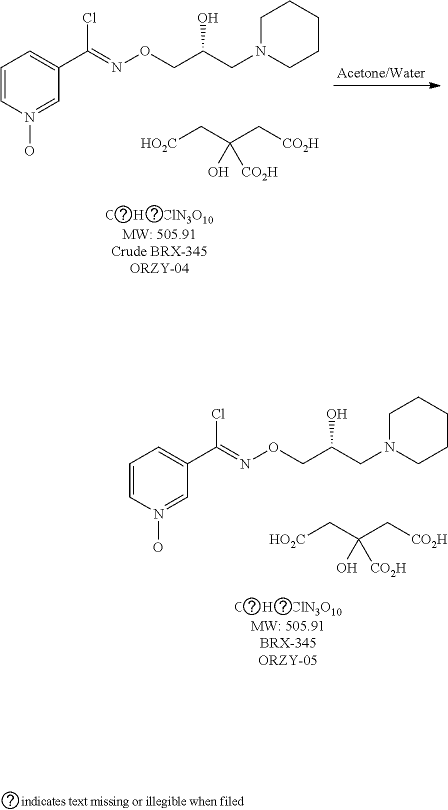

### Step 4: Overview of Process for Preparing BRX-345 (ORZY-OS)

The disclosed methods contribute to control of both the chiral purity (i.e., the enantiomeric excess) of N-{[(2R)-2-hydroxy-3-piperidin-1-ylpropyl]oxy}pyridine-3-carboximidoyl chloride 1-oxide citrate is above the threshold set by the regulatory authorities, and that the ultra-pure composition is essentially free of previously identified by-products, such N-nitrosopiperidine.

The chiral purity of the ultra-pure composition comprising arimoclomol citrate is resultant of the chiral resolution step, i.e. the method according to “Step 2” of the present disclosure.

The present disclosure provides a correlation between the cooling rate of the crude reaction mixture in Step 2 and the chiral purity of ORZY-03. The chiral purity of ORZY-03 obtained in Step 2 is retained toward the end-product, but may be further enhanced by re-crystallization.

### III. COMPOSITIONS COMPRISING ARIMOCLOMOL AND PHARMACEUTICALLY ACCEPTABLE SALTS THEREOF

In an embodiment, the present disclosure provides compositions comprising a therapeutically effective amount of arimoclomol or a pharmaceutically acceptable salt thereof.

“Pharmaceutical composition,” as used herein, means a composition comprising at least one active pharmaceutical ingredient (API) and, optionally, one or more excipients as defined herein. “Excipients” as used herein, means pharmaceutically inert compounds which may include one or more of the following types: antiadherents, binders, coatings, disintegrants, gel forming agents, fillers, flavors and colors, glidants, lubricants, preservatives, sorbents and sweeteners.

“Therapeutically effective amount” as it relates to the present technology means an amount that has a pharmacological effect. “Pharmaceutically effective amount” may be used interchangeably with “therapeutically effective amount,” which as used herein, means an amount effective for treating a disease or condition.

A “therapeutically acceptable salt” as used herein is a pharmaceutically acceptable salt of arimoclomol in the composition of the present technology, which, when used in a therapeutically effective amount, is effective for treating a disease, condition, or syndrome.

In certain embodiments, the therapeutically effective amount of arimoclomol ranges from 0.1 mg/kg/day to about 30 mg/kg/day. Alternatively, the therapeutically effective amount ranges from about 0.1 mg/day to about 500 mg/day. In certain embodiments, the therapeutically effective amount is selected from 141 mg, 186 mg, 273 mg, or 372 mg per day. In an alternative embodiment, the therapeutically effective amount is selected from 93 mg, 141 mg, 186 mg, or 372 mg per day. In an alternative embodiment, the therapeutically effective amount is selected from 93 mg, 141 mg, 186 mg, 273 mg, or 372 mg per day.

In yet another embodiment, the present disclosure provides compositions comprising arimoclomol or a pharmaceutically acceptable salt thereof, wherein the composition is formulated for parenteral administration. “Parenteral administration” as it relates to the present technology, means administration by injection, infusion, intravenous such as through a drip line. Parenteral administration may also mean through dermal absorptions such as from a skin patch.

In yet another embodiment, the present disclosure provides compositions comprising arimoclomol or a pharmaceutically acceptable salt thereof, wherein the composition is formulated for oral administration. In a still further embodiment, the composition is a solid oral dosage formulation. “Solid oral dosage formulation” as it relates to the present technology means dosage forms that include but are not limited to sublingual, gummy, chewable tablet, rapidly dissolving tablet, tablet, capsule, caplet, troche, lozenge, powder, oral thin film (OTF), oral strip, rectal film, or suppository. In some embodiments, the dosage forms are to be administered orally. Preferred oral administration forms are capsule, tablet, solutions and OTF. Solid oral dosage formulations can optionally include one or more of the following types of excipients: antiadherents, binders, coatings, disintegrants, gel forming agents, fillers, flavors and colors, glidants, lubricants, preservatives, sorbents and sweeteners.

Other compounds which may be included by admixture are, for example, medically inert ingredients, e.g., solid and liquid diluents, such as lactose, dextrose, saccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate, hydrogenated oils, sodium stearyl fumarate, and/or polyethylene glycols; gelling agents such as colloidal clays, polyethylene oxide, hydroxypropyl methyl cellulose, or carbomers; thickening agents such as gum tragacanth or sodium alginate, binding agents such as starches, arabic gums, gelatin, methylcellulose, carboxymethylcellulose or polyvinylpyrrolidone (povidone); disintegrating agents such as starch, alginic acid, alginates, crospovidone, or sodium starch glycolate; effervescing mixtures; dyestuff; sweeteners; wetting agents such as lecithin, polysorbates, poloxamer, sorbitan monoesters, glyceryl monooleates, or laurylsulfates; and other therapeutically acceptable accessory ingredients, such as humectants, preservatives, buffers and antioxidants, which are known additives for such formulations. In a still further embodiment, the solid oral dosage formulation is selected from the group consisting of a sublingual, a gummy, a chewable tablet, a rapidly dissolving tablet, a tablet, a capsule, a caplet, a troche, a lozenge, an oral powder, a solution, a thin strip, an oral thin film (OTF), an oral strip, and a rectal film.

Alternatively, the oral dosage formulation is liquid oral dosage formulation. “Liquid oral dosage form” as it relates to the present technology means solutions, syrups, emulsions, or suspensions. The syrups may contain as carrier, for example, saccharose or saccharose with glycerol and/or mannitol and/or sorbitol. In particular a syrup for diabetic subjects can contain as carriers only products, for example sorbitol, which does not metabolize to glucose, or which metabolizes to only a very small amount of glucose. The suspensions and the emulsions may contain a carrier, for example a natural gum, agar, sodium alginate, pectin, methylcellulose, carboxymethylcellulose or polyvinyl alcohol. Liquid oral formulations of the present technology can also be included in a solution, a suspension or a slurry in an aqueous liquid or a non-aqueous liquid. The formulation can be an emulsion, such as an oil-in-water liquid emulsion or a water-in-oil liquid emulsion. The oils can be administered by adding the purified and sterilized liquids to a prepared enteral formula, which is then placed in the feeding tube or gastric tube of a subject who is unable to swallow. In a still further embodiment, the liquid oral dosage formulation is selected from the group consisting of syrups, emulsions, solutions, and suspensions.

In a still further embodiment, the present disclosure provides for a composition comprising a therapeutically effective amount of a compound of Formula I, wherein the composition is formulated for extended release of the compound of Formula I. “Formulated for extended release,” as used herein, means a composition comprising one or more extended release excipients. “Extended release excipients,” as used herein, one or more materials used to prolong the release of an active pharmaceutical into the blood compared to a formulation lacking said materials.

Common extended release excipients include, but are not limited to hydrophilic polymers such as hydroxypropyl methylcellulose (HPMC), hydroxy propyl cellulose (HPC), sodium alginate, carbomers, and xanthan gum; hydrophobic polymers such as ethylcellulose, cellulose acetate, or polyvinyl acetate; coating agents such as Eudragit® polymers, ethylcellulose, polyvinyl acetate, or shellac; osmotic agents such as sodium chloride, mannitol, lactose, or polyethylene oxide (PEO); lubricants and glidants such as magnesium stearate, talc, or colloidal silicon dioxide; plasticizers such as triethyl citrate, diethyl phthalate, or polyethylene glycol (PEG); pH-dependent polymers such as Eudragit® polymer, cellulose acetate phthalate (CAP), Hypromellose phthalate (HPMCP); and/or waxes and lipids such as carnauba wax, beeswax, hydrogenated castor oil, or glyceryl behenate.

In certain embodiments, the composition is formulated in a unit dose form. “Unit dose form” as it related to the present technology means a single entity of a solid therapeutic dosage form (e.g., 1 capsule, 1 tablet) or a single volume dispensed from a non-solid dosage form (e.g., 5 mL of a liquid or syrup). When the unit dose is administered once per day, the dosing is “q.d.” dosing. In certain other embodiments, unit dose forms are administered multiple times per day, such a “b.i.d.” (twice daily) or “t.i.d.” (three times per day). In certain embodiments, the unit dose form is selected from 31 mg t.i.d., 47 mg t.i.d., 62 mg t.i.d, 93 mg t.i.d., or 124 mg t.i.d.; alternatively, the unit dose form is selected from 47 mg t.i.d., 62 mg t.i.d, 93 mg t.i.d., or 124 mg t.i.d.

### IV. METHODS OF TREATMENT

In one embodiment, the present disclosure provides a method of increasing removal of lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

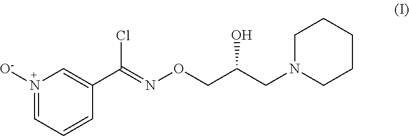

or a pharmaceutically acceptable salt thereof, wherein increasing the activation of the at least one transcription factor increases the removal of lysosomal cholesterol from the cells of the patient relative to a patient not administered a therapeutically effective amount of the compound of Formula I, or a pharmaceutically acceptable salt thereof.

“Increasing removal of lysosomal cholesterol,” as used herein, means increasing the amount of lysosomal cholesterol removed from the lysosome, or, in an alternative, increasing the rate at which cholesterol is removed from the lysosome, as compared to a patient who is not being treated with a therapeutically effective amount of a compound of Formula I.

“Lysosomal storage disorder” or “Lysosomal Storage Disorders” as it related to the present technology means a disease or disorder associated with reduced lysosomal function. Lysosomal storage diseases (LSDs) are a group of approximately 40 rare inherited metabolic disorders that result from defects in lysosomal function. LSDs are caused by lysosomal dysfunction usually as a consequence of deficiency of a single enzyme required for the metabolism of lipids, glycoproteins or mucopolysaccharides. Although each disorder results from different gene mutations that translate into a deficiency in enzyme activity, they all share a common biochemical characteristic-all lysosomal disorders originate from an abnormal accumulation of substances inside the lysosome. Individually LSDs occur with incidences of less than 1:100000, however, as a group the incidence is about 1:5000-1:10000. Most of these disorders are autosomal recessively inherited, however a few are X-linked recessively inherited, such as Fabry disease.

The lysosomal storage diseases are generally classified by the nature of the primary stored material involved, and can be broadly broken into the following: lipid storage disorders (or lipidoses), mainly sphingolipidoses (including Gaucher's and Niemann-Pick diseases); gangliosidosis (including Tay-Sachs disease); leukodystrophies; mucopolysaccharidoses (including Hunter syndrome and Hurler disease); glycoprotein storage disorders (glycoproteinosis); and nucolipidoses. Depending on the severity of the disease, patients either die at a young and unpredictable age, many within a few months or years of birth, whereas others survive into early adulthood finally succumbing to the various pathologies of their particular disorder. The symptoms of LSD vary, depending on the particular disorder and can be mild to severe. They can include developmental delay, movement disorders, seizures, dementia, deafness and/or blindness. Some people with LSD have enlarged livers (hepatomegaly) and enlarged spleens (splenomegaly), pulmonary and cardiac problems, and abnormal bone growth. The majority of patients are initially screened by an enzyme assay, which is the most efficient method to arrive at a definitive diagnosis. In some families where the disease-causing mutation(s) is known and in certain genetic isolates, mutation analysis may be performed. As there may be numerous different mutations, sequencing of the gene encoding the particular affected enzyme is sometimes necessary to confirm the diagnosis. Prenatal diagnosis may be useful when there is a known genetic risk factor. FEB and TFE3 activate expression of genes responsible for lysosomal protein synthesis. Lysosomes are organelles responsible for removing waste from the cell. Genes activated by TFEB and TFE3 are collectively known as the CLEAR network of genes that include NPC1. In cells unaffected by NPC, normally assembled and maturated proteins migrate to the lysosomal membrane where they play a critical role in transporting cholesterol and other lipids out of the lysosome. In healthy cells, this process supports elimination of cellular waste (autophagy) and promotes healthy neuronal function.

“Nieman-Pick Disease” or “NP” as it relates to the present technology means a group of rare genetic diseases of varying severity. These are inherited metabolic disorders in which sphingomyelin accumulates in lysosomes in cells of many organs. NP types A, A/B, and B are caused by mutations in the SMPD1 gene, which causes a deficiency of an acid sphingomyelinase (ASM). NP type C is now considered a separate disease, as SMPD1 is not involved, and there is no deficiency in ASM. State. Arimoclomol targets NPC etiology by both NPC1-independent and NPC1-dependent pathways: (1) via the NPC1-independent pathway, arimoclomol upregulates expression of all CLEAR genes, thereby mitigating the deleterious effects of impaired cholesterol trafficking and improving overall cell health; (2) via the NPC1-dependent pathway, CLEAR gene upregulation increases production of the NPC1 protein. Though still mutated, overproducing the reduced function protein improves lysosomal function and cholesterol elimination.

“Nieman-Pick Disease Type C” or “NPC” as it relates to the present technology means a rare progressive genetic disorder characterized by an inability of the body to transport cholesterol and other fatty substances (lipids) inside of cells. This leads to the abnormal accumulation of these substances within various tissues of the body, including brain tissue.

“Nieman-Pick Type C Protein 1” or “NPC1” as it relates to the present technology means the gene encoding the Niemann-Pick type C protein 1, also referred to in the art as the NPC intracellular cholesterol transporter 1. The term “NPC1” refers to the protein product of the NPC1 gene. In patients with NPC, the NPC1 protein has substantially diminished quantity and functionality because mutations in NPC genes prevent most NPC1 protein from completing assembly and maturation in the endoplasmic reticulum and Golgi apparatus. Furthermore, the small amount of NPC1 protein that does localize in the lysosome is thought to be less effective, leading to cholesterol accumulation and neuronal death.

“NPC1^{-/-}” as it relates to the present technology means a mouse model that possesses a null allele mutation in the NPC1 gene which results in expression of a non-functional mutant NPC1 protein.

“NPC1^{nmf164}” as it relates to the present technology means a mouse model that possesses a point mutation in the NPC1 genes which results in expression of a mutant NPC1 protein with reduced function compared to wild-type NPC1 protein.

“NPC1-dependent” as it relates to the present technology means a biological pathway that involves the NPC1 gene and/or the NPC1 protein.

“NPC1-independent” as it relates to the present technology means a biological pathway that does not involve the NPC1 gene or NPC1 protein.

“Transcription Factor EB” as it relates to the present technology means protein that in humans is encoded by the TFEB gene. TFEB is a master gene for lysosomal biogenesis.[7] It encodes a transcription factor that coordinates expression of lysosomal hydrolases, membrane proteins and genes involved in autophagy. Upon nutrient depletion and under aberrant lysosomal storage conditions such as in lysosomal storage diseases, TFEB translocate from the cytoplasm to the nucleus, resulting in the activation of its target genes.

“Transcription Factor E3” as it relates to the present technology means a protein that in humans is encoded by the TFE3 gene. TFE3, a member of the helix-loop-helix family of transcription factors, binds to the mu-E3 motif of the immunoglobulin heavy-chain enhancer and is expressed in many cell types.

“Miglustat,” as used herein, means 1,5-(butylimino)-1,5-dideoxy-D-glucitol, N-butyldeoxynojirimycin, (2R,3R,4R,5S)-1-butyl-2-(hydroxymethyl)-3,4,5-piperidinetriol; (2R,3R,4R,5S)-1-butyl-2-(hydroxymethyl)piperidine-3,4,5-triol; 1,5-(butylimino)-1,5-dideoxy-D-glucitol; 1,5-dideoxy-1,5-N-butylimino-D-glucitol; 3,4,5-piperidinetriol, 1-butyl-2-(hydroxymethyl)-, (2R-(2alpha,3beta,4alpha,5beta))-; 3,4,5-Piperidinetriol, 1-butyl-2-(hydroxymethyl)-, (2R,3r,4R,5S)-; Brazaves; BuDNJ; butyldeoxynojirimycin; D-glucitol, 1,5-(butylimino)-1,5-dideoxy-; miglustatum; N-(n-butyl)-1,5-dideoxy-1,5-imino-D-glucitol; N-(n-butyl)-1-deoxynojirimycin; N-(n-butyl)deoxynojirimycin; N-Bu-DNJ; n-butyl deoxynojirimycin; n-butyl dnj; N-butyl-1-deoxynojirimycin; N-butyldeoxynojirimycin; N-butyl-deoxynojirimycin; N-Butyl-DNJ; N-butylmoranoline; Opfolda; Vevesca; Yargesa; Zavesca and/or NB-DNJ.

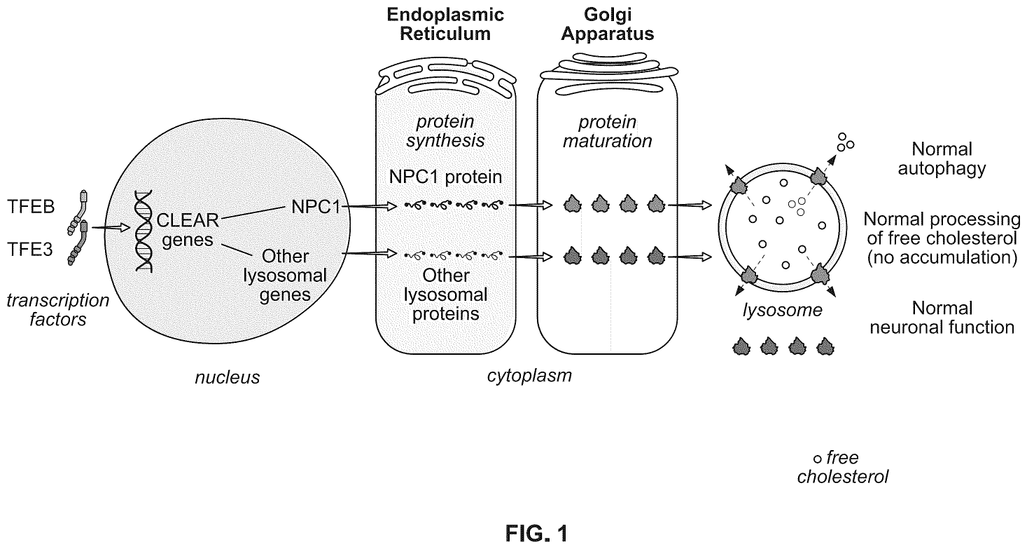

FIG. 1 depicts the relationship between the CLEAR gene network, NPC1 expression, and cholesterol trafficking in a healthy patient. Healthy lysosomal function requires a number of proteins regulated by TFEB and TFE3 to maintain homeostasis. The ratio of cytosolic and nuclear pools of these transcription factors determines the degree of their activation whereby translocation to the nucleus correlates with increased gene expression. Both transcription factors promote expression of genes responsible for lysosomal protein synthesis and other autophagy related functions. Lysosomes are organelles responsible for removing waste materials from the cell. Genes activated by TFEB and TFE3 are collectively known as the CLEAR network of genes. In cells unaffected by NPC, normally assembled and maturated NPC protein migrates to the lysosomal membrane where they play a critical role in transporting cholesterol out of the lysosome. In healthy cells, this process supports elimination of waste (autophagy) to maintain healthy cellular function.

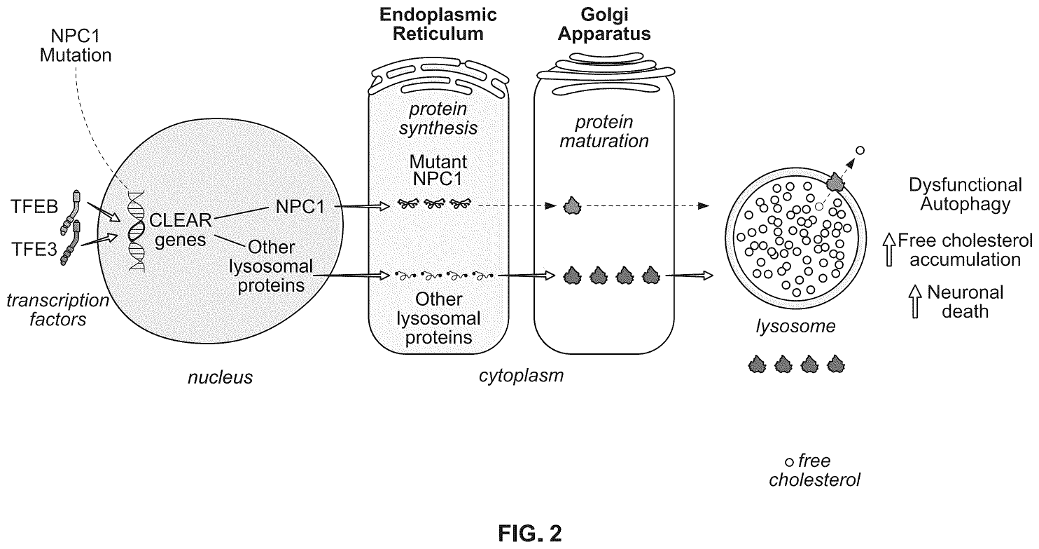

FIG. 2 shows the same relationship in NPC patient cells. In patients with NPC missense mutations, the NPC1 protein typically has diminished quantity and functionality because mutations in the NPC1 genes prevent most NPC1 protein from completing assembly and maturation in the endoplasmic reticulum (ER) and Golgi apparatus. The degree of early degradation and residual protein function depends on the genotype. Nonetheless, a large portion of mutations including the most common I1061T variant remain functional but are subject to early degradation by quality control checks in the ER. As a result, only very small amounts of NPC1 protein are being incorporated into lysosomal membranes, leading to cholesterol accumulation and neuronal death.

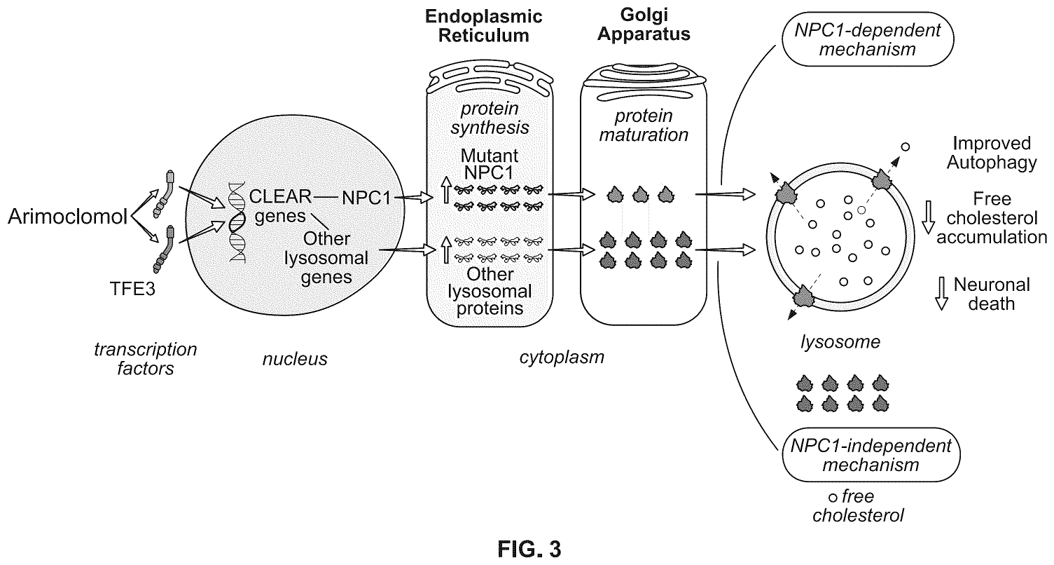

FIG. 3 depicts one route by which arimoclomol targets NPC etiology by both NPC1-dependent and NPC1-independent pathways. For NPC1-dependent patients, CLEAR gene upregulation increases production of the NPC1 protein. Though still mutated, overproducing the protein that typically has residual function if trafficked properly improves lysosomal function through increased export of cholesterol. For NPC1-independent patients, arimoclomol upregulates expression of CLEAR genes, thereby rescuing impaired autophagy flux to improve overall cell health and lifespan.

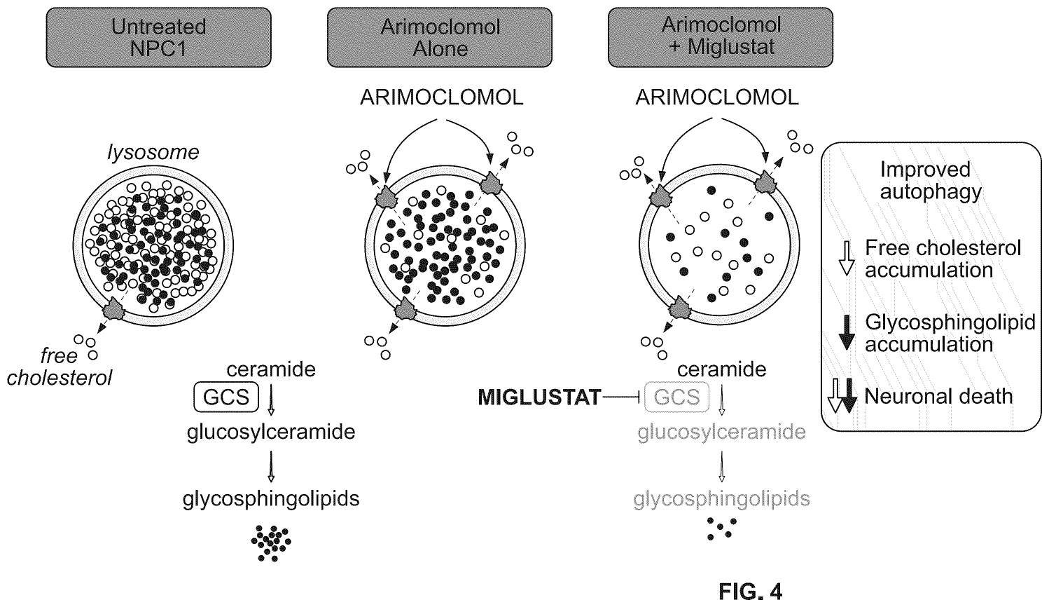

FIG. 4 illustrates how co-administration of miglustat with arimoclomol further improves cholesterol trafficking. Miglustat acts to block glucosylceramide synthase (GCS) which reduces the production of glycosylceramine and by extension glycophingolipids, which results in an overall lowering of lipid build up in the cell.

In yet another embodiment, the present disclosure provides a method of increasing removal of lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I or a pharmaceutically acceptable salt thereof, wherein the lysosomal cholesterol is unesterified cholesterol. “Unesterified cholesterol” as it relates to the present technology means “free cholesterol,” or cholest-5-en-3β-ol. “Free cholesterol,” as it relates to the present technology means cholesterol not attached to a fatty acid. “Free Cholesterol” as it relates to the present technology includes, and may be used interchangeably with “unesterified cholesterol” or “cholesterol.”

In yet another embodiment, the present disclosure provides a method of increasing removal of lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I or a pharmaceutically acceptable salt thereof, wherein the patient has a disease associated with impaired cholesterol trafficking. In certain embodiments, the disease associated with impaired cholesterol trafficking is NPC (Niemann-Pick, Type C disease). Alternatively, the NPC disease is selected from the group consisting of NPC, Type 1 (NPC-1) and NPC Type 2 (NPC2). In one embodiment, the NPC disease is NPC1.

In yet another embodiment, the present disclosure provides a method of increasing removal of lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I or a pharmaceutically acceptable salt thereof, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. Alternatively, the pharmaceutically acceptable salt is citrate. In one embodiment, the compound of Formula I is arimoclomol citrate.

In yet another embodiment, the present disclosure provides a method of increasing removal of lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I or a pharmaceutically acceptable salt thereof, wherein the compound having structure of Formula I is N-[(2R,Z)-2-hydroxy-3-(1-piperidyl)propoxy]pyridine-3-carboximidoyl chloride, 1-oxide citrate.

In yet another embodiment, the present disclosure provides a method of increasing removal of lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I or a pharmaceutically acceptable salt thereof, wherein the therapeutically effective amount of the compound having the structure of Formula I is a daily dosage of about 1 to about 30 mg/kg/day; alternatively, the daily dosage is 30 mg/kg/day. In a still further embodiment, the therapeutically effective amount ranges from about 0.1 mg to about 600 mg per day; alternatively, the therapeutically effective amount ranges from about 50 mg to about 500 mg per day; alternatively, from about 100 mg to about 400 mg per day. In certain embodiments, the therapeutically effective amount is selected from about 93 mg per day, about 141 mg per day, about 189 mg per day, about 279 mg per day, or about 372 mg per day. In an alternative embodiment, the therapeutically effective amount is selected from about 93 mg per day, about 141 mg per day, about 189 mg per day, about 279 mg per day, or about 372 mg per day.

In yet another embodiment, the present disclosure provides a method of increasing removal of lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I or a pharmaceutically acceptable salt thereof, wherein the daily dosage is administered in about 1-3 unit doses per day. In one embodiment, the daily dosage is administered in 3 unit doses per day.

In yet another embodiment, the present disclosure provides a method of increasing removal of lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I or a pharmaceutically acceptable salt thereof, wherein the therapeutically effective amount is selected from 47 mg t.i.d., 63 mg t.i.d., 93 mg t.i.d., or 124 mg t.i.d. “T.i.d,” as used herein, means three times per day. In an alternative embodiment, the therapeutically effective amount is selected from 31 mg t.i.d., 47 mg t.i.d., 63 mg t.i.d., 93 mg t.i.d., or 124 mg t.i.d.

In yet another embodiment, the present disclosure provides a method of increasing removal of lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I or a pharmaceutically acceptable salt thereof, wherein the composition is combined with soft food prior to administration. In a further embodiment, the composition is dispersed in water prior to administration. The composition may be dispersed in water by, for example, opening a capsule and emptying the contents of the capsule into the water.

In yet another embodiment, the present disclosure provides a method of increasing removal of lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I or a pharmaceutically acceptable salt thereof, wherein the composition is an oral dosage formulation. In a further embodiment, the oral dosage formulation is a solid oral dosage formulation. In certain embodiments, the solid oral dosage is selected from the group consisting of tablets, capsules, caplets, films, and powders and may further one or more excipients.

Alternatively, the oral dosage formulation is a liquid oral dosage formulation. In yet another embodiment, the liquid oral dosage formulation comprises 3.1 mg/mL arimoclomol. In certain embodiments, the liquid oral dosage formulation further comprises a thickening agent. In a still further embodiment, the liquid oral dosage is formulated for administration via a feeding tube or gastric tube.

In yet another embodiment, the present disclosure provides a method of increasing removal of lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I or a pharmaceutically acceptable salt thereof, wherein the composition, wherein the composition is formulated for parenteral administration. Examples of parenteral administration include, but are not limited to, subcutaneous, intramuscular, intravenous, and intrathecal.

For patients with renal impairment, the method further comprises the steps of determining the Estimated Glomerular Filtration Rate (eGFR) of the subject/patient; and modifying the therapeutically effective amount based on whether the patient has mild to moderate renal impairment or moderate to severe renal impairment. “Moderate to severe renal impairment,” as used herein means a eGFR ranging from 15 mL/min to 50 mL/min. The therapeutically effective amount of the compound having a structure of Formula I, or a pharmaceutically acceptable salt thereof, for patients with moderate to severe renal impairment is about 1 to about 15 mg/kg/day. In one embodiment, the therapeutically effective amount is 15 mg/kg/day. In certain embodiments, the therapeutically effective amount is administered in 2 unit doses per day.

In yet another embodiment, the present disclosure provides a method of removing endosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

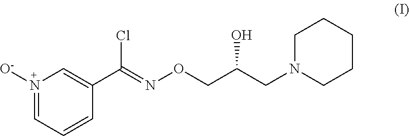

or a pharmaceutically acceptable salt thereof, where increasing the activation of the at least one transcription factor increases the removal of endosomal cholesterol from the cells of the patient relative to a patient not administered a therapeutically effective amount of the compound of Formula I, or a pharmaceutically acceptable salt thereof. In an alternative embodiment, the method increases the removal of endosomal cholesterol from cells.

“Increasing removal of endosomal cholesterol,” as used herein, means increasing the amount of endosomal cholesterol removed from the endosome, or, in an alternative, increasing the rate at which cholesterol is removed from the endosome, as compared to a patient who is not being treated with a therapeutically effective amount of a compound of Formula I.

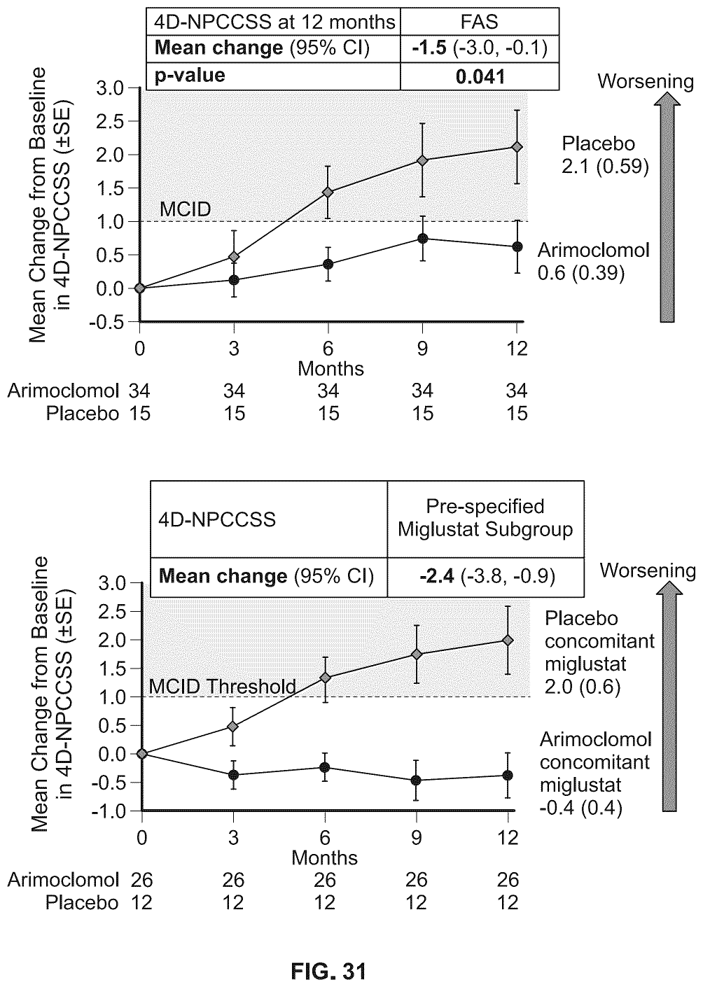

FIG. 31 shows the reduction of unesterified cholesterol in NPC patient fibroblasts with increasing arimoclomol dosage determined by Filipin Staining. “Filipin Staining,” as used herein means a technique used in cell biology and microscopy to detect and visualize free cholesterol in cell membranes. Depending on the specific genotype and the functionality of the resulting mutant protein, this greater availability of trafficking competent NPC1 protein that can reach the late endosomal/lysosomal membranes results in various degrees of improved clearance of unesterified cholesterol.

In yet another embodiment, the present disclosure provides a method of removing endosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, pharmaceutically acceptable salt thereof, wherein the endosomal cholesterol is unesterified cholesterol.

In yet another embodiment, the present disclosure provides a method of removing endosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, pharmaceutically acceptable salt thereof, wherein the patient has a disease associated with impaired cholesterol trafficking. In a still further embodiment, the disease is NPC (Niemann-Pick, Type C disease). In certain embodiments, the NPC disease is selected from the group consisting of NPC, Type 1 (NPC-1) and NPC Type 2 (NPC2). Alternatively, the NPC disease is NPC1.

In yet another embodiment, the present disclosure provides a method of removing endosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, pharmaceutically acceptable salt thereof, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a still further embodiment, the pharmaceutically acceptable salt is citrate; alternatively, the compound of Formula I is arimoclomol citrate.

In yet another embodiment, the present disclosure provides a method of removing endosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, pharmaceutically acceptable salt thereof, wherein the compound having structure of formula (I) is N-[(2R,Z)-2-hydroxy-3-(1-piperidyl)propoxy]pyridine-3-carboximidoyl chloride, 1-oxide citrate.

In yet another embodiment, the present disclosure provides a method of removing endosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or pharmaceutically acceptable salt thereof, wherein the therapeutically effective amount of the compound having the structure of Formula I is a daily dosage of about 1 to about 30 mg/kg/day; alternatively, the daily dosage is 30 mg/kg/day. In a still further embodiment, the therapeutically effective amount ranges from about 0.1 mg to about 600 mg per day; alternatively, the therapeutically effective amount ranges from about 50 mg to about 500 mg per day; alternatively, from about 100 mg to about 400 mg per day. In certain embodiments, the therapeutically effective amount is selected from about 141 mg per day, about 189 mg per day, about 279 mg per day, or about 372 mg per day. In an alternative embodiment, the therapeutically effective amount is selected from about 93 mg per day, about 141 mg per day, about 189 mg per day, about 279 mg per day, or about 372 mg per day.

In yet another embodiment, the present disclosure provides a method of removing endosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or pharmaceutically acceptable salt thereof, wherein the daily dosage is administered in about 1-3 unit doses per day; alternatively, the daily dosage is administered in 3 unit doses per day.

In yet another embodiment, the present disclosure provides a method of removing lysosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I or a pharmaceutically acceptable salt thereof, wherein the therapeutically effective amount is selected from 47 mg t.i.d., 63 mg t.i.d., 93 mg t.i.d., or 124 mg t.i.d. “T.i.d,” as used herein, means three times per day. In an alternative embodiment, the therapeutically effective amount is selected from 31 mg. t.i.d., 47 mg t.i.d., 63 mg t.i.d., 93 mg t.i.d., or 124 mg t.i.d.

In yet another embodiment, the present disclosure provides a method of removing endosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or pharmaceutically acceptable salt thereof, wherein the composition is combined with soft food prior to administration; alternatively, the composition is dispersed in water prior to administration.

In yet another embodiment, the present disclosure provides a method of removing endosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or pharmaceutically acceptable salt thereof, wherein the composition is an oral dosage formulation. In a further embodiment, the oral dosage formulation is a solid oral dosage formulation. In a still further embodiment, the solid oral dosage is selected from the group consisting of tablets, capsules, caplets, films, and powders. In certain embodiments, the solid oral dosage form further comprises one or more excipients.

In yet another embodiment, the present disclosure provides a method of removing endosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or pharmaceutically acceptable salt thereof, wherein the oral dosage formulation is a liquid oral dosage formulation In a still further embodiment, the liquid oral dosage formulation further comprises a thickening agent; alternatively, the liquid oral dosage formulation is formulated for administration via a feeding tube or gastric tube.

In yet another embodiment, the present disclosure provides a method removing endosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or pharmaceutically acceptable salt thereof, wherein the composition is formulated for parenteral administration.

For patients with renal impairment, the method further comprises the steps of determining the Estimated Glomerular Filtration Rate (eGFR) of the subject/patient; and modifying the therapeutically effective amount based on whether the patient has mild to moderate renal impairment or moderate to severe renal impairment. “Moderate to severe renal impairment,” as used herein means a eGFR ranging from 15 mL/min to 50 mL/min. The therapeutically effective amount of the compound having a structure of Formula I, or a pharmaceutically acceptable salt thereof, for patients with moderate to severe renal impairment is about 1 to about 15 mg/kg/day. In one embodiment, the therapeutically effective amount is 15 mg/kg/day. In certain embodiments, the therapeutically effective amount is administered in 2 unit doses per day.

In an embodiment, the present disclosure provides a method of increasing cellular production of NPC1 protein in a patient with a mutant Niemann-Pick type-C Protein 1 (NPC1) with decreased functionality compared to wild-type NPC1 protein, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

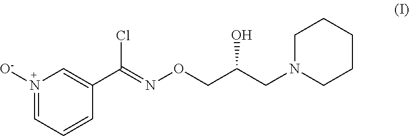

or a pharmaceutically acceptable salt thereof, where increasing the activation of the at least one transcription factor increases cellular production of the mutant NPC1 protein having reduced functionality, relative to a patient not administered a therapeutically effective amount of the compound of Formula I, or a pharmaceutically acceptable salt thereof.

“Increasing cellular production of NPC1 protein in a patient with a mutant Niemann-Pick type-C Protein 1 (NPC1) with decreased functionality,” as used herein, means increasing the amount of NPC1 produced by the cell, or, in an alternative, increasing the rate at which NPC1 protein is produced, as compared to a patient having a mutant Niemann-Pick type-C Protein 1 (NPC1) with decreased functionality compared to wild-type NPC1 who is not being treated with a therapeutically effective amount of a compound of Formula I.

In yet another embodiment, the present disclosure provides a method of increasing cellular production of NPC1 protein in a patient with a mutant Niemann-Pick type-C Protein 1 (NPC1) with decreased functionality compared to wild-type NPC1 protein, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the disease or disorder resulting in mutant Niemann-Pick type C Protein 1 (NPC1) with deceased functionality is Niemann-Pick disease. In a still further embodiment, the Niemann-Pick disease is selected from the group consisting of NPC-1 and NPC-2; alternatively, the NPC disease is NPC1.

In yet another embodiment, the present disclosure provides a method of increasing cellular production of NPC1 protein in a patient with a mutant Niemann-Pick type-C Protein 1 (NPC1) with decreased functionality, compared to wild-type NPC1 protein, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a still further embodiment, the pharmaceutically acceptable salt is citrate; alternatively, the compound of Formula I is arimoclomol citrate.

In yet another embodiment, the present disclosure provides a method of increasing cellular production of NPC1 protein in a patient with a mutant Niemann-Pick type-C Protein 1 (NPC1) with decreased functionality compared to wild-type NPC1 protein, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the compound having structure of formula (I) is N-[(2R,Z)-2-hydroxy-3-(1-piperidyl)propoxy]pyridine-3-carboximidoyl chloride, 1-oxide citrate.

In yet another embodiment, the present disclosure provides a method of increasing cellular production of NPC1 protein in a patient with a mutant Niemann-Pick type-C Protein 1 (NPC1) with decreased functionality compared to wild-type NPC1 protein, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the therapeutically effective amount of the compound having the structure of Formula I is a daily dosage of about 1 to about 30 mg/kg/day; alternatively, the daily dosage is 30 mg/kg/day. In an alternative embodiment, the therapeutically effective amount ranges from about 0.1 mg to about 600 mg per day; alternatively the therapeutically effective amount ranges from about 50 mg to about 500 mg per day; alternatively, from about 100 mg to about 400 mg per day. In certain embodiments, the therapeutically effective amount is selected from about 141 mg per day. About 189 mg per day, about 279 mg per day, or about 372 mg per day. In yet another alternative embodiment, the therapeutically effective amount is selected from about 93 mg per day, about 141 mg per day, about 189 mg per day, about 279 mg per day, or about 372 mg per day.

In yet another embodiment, the present disclosure provides a method of increasing cellular production of NPC1 protein in a patient with a mutant Niemann-Pick type-C Protein 1 (NPC1) with decreased functionality compared to wild-type NPC1, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the daily dosage is administered in about 1-3 unit doses per day. In a still further embodiment, the daily dosage is administered in 3 unit doses per day. In an embodiment, the daily dosage is selected from 31 mg t.i.d., 47 mg t.i.d., 62 mg t.i.d., 93 mg t.i.d., or 124 mg t.i.d.; alternatively, the daily dosage is selected from 47 mg t.i.d., 62 mg t.i.d., 93 mg t.i.d., or 124 mg t.i.d..

In yet another embodiment, the present disclosure provides a method of increasing cellular production of NPC1 protein in a patient with a mutant Niemann-Pick type-C Protein 1 (NPC1) with decreased functionality compared to wild-type NPC1 protein, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is combined with soft food prior to administration; alternatively, the composition is dispersed in water prior to administration.

In yet another embodiment, the present disclosure provides a method of increasing cellular production of NPC1 protein in a patient with a mutant Niemann-Pick type-C Protein 1 (NPC1) with decreased functionality compared to wild-type NPC1 protein, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is an oral dosage formulation. In a still further embodiment, the oral dosage formulation is a solid oral dosage formulation. In certain embodiments, the solid oral dosage is selected from tablets, capsules, caplets, films, and powders and may further comprises one or more excipients.

In yet another embodiment, the present disclosure provides a method of increasing cellular production of NPC1 protein in a patient with a mutant Niemann-Pick type-C Protein 1 (NPC1) with decreased functionality compared to wild-type NPC1 protein, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the oral dosage formulation is a liquid oral dosage formulation. In a still further embodiment, the liquid oral dosage formulation comprises 3.1 mg/mL arimoclomol. In a still further embodiment, the liquid oral dosage formulation further comprises a thickening agent; alternatively, the liquid oral dosage formulation is formulated for administration via a feeding tube or gastric tube.

In yet another embodiment, the present disclosure provides a method of increasing cellular production of NPC1 protein in a patient with a mutant Niemann-Pick type-C Protein 1 (NPC1) with decreased functionality compared to wild-type NPC1 protein, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is formulated for parenteral administration.

For patients with renal impairment, the method further comprises the steps of determining the Estimated Glomerular Filtration Rate (eGFR) of the subject/patient; and modifying the therapeutically effective amount based on whether the patient has mild to moderate renal impairment or moderate to severe renal impairment. “Moderate to severe renal impairment,” as used herein means a eGFR ranging from 15 mL/min to 50 mL/min. The therapeutically effective amount of the compound having a structure of Formula I, or a pharmaceutically acceptable salt thereof, for patients with moderate to severe renal impairment is about 1 to about 15 mg/kg/day. In one embodiment, the therapeutically effective amount is 15 mg/kg/day. In certain embodiments, the therapeutically effective amount is administered in 2 unit doses per day.

In an embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

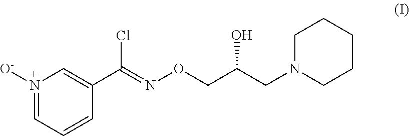

or a pharmaceutically acceptable salt thereof, where increasing the activation of the at least one transcription factor decreases at least one 4D-NPCCSS domain score, wherein the at least one 4D-NPCCSS domain is selected from ambulation, fine motor skills, and speech, relative to a patient not administered a therapeutically effective amount of the compound of Formula I, or a pharmaceutically acceptable salt thereof.

“Improving at least one 4D-NPCCSS scale domain score,” as used herein, means decreasing at least one domain score from the 4D-NPCCSS test relative to a patient not administered a therapeutically effective amount of the compound of Formula I, or a pharmaceutically acceptable salt thereof. In a non-limiting example, a 1-point decrease in the ambulation domain score is an improvement in at least one 4D-NPCCSS scale domain score.

“Four-domain Niemann-Pick disease type C Clinical Severity Scale” and/or “4D-NPCCSS” as it relates to the present technology means a composite clinical severity scale (hereafter “NPCCSS”; see Yanjanin et al.). A full “17-domain NPCCSS score” incorporates clinical signs and symptoms in nine major (ambulation, cognition, eye movement, fine motor, hearing, memory, seizures, speech, swallowing) and eight minor (auditory brainstem response, behavior, gelastic cataplexy, hyperreflexia, incontinence, narcolepsy, psychiatric, respiratory problems) domains to determine a score which describes the severity of the subject's NPC progression (a higher score, the more progressed/severe the disease is). An abridged “5-domain NPCCSS score” is successfully used by clinicians, and incorporates clinical signs and symptoms from the major domains of ambulation, cognition, fine motor, speech and swallowing (see Cortina-Borja). The updated “4-domain NPCCSS score” is used based on Ambulation, Fine Motor Skills, Speech, and updated Swallow criteria. To score the NPCCSS, clinicians evaluated the patient's clinical symptoms and assigned a score of 0 to 5 in each domain, based on defined criteria. The 4D-NPCCSS total score ranges from 0 to 20 points, with higher scores representing more severe clinical impairment. Strong correlations between each of the four domains of the 4D-NPCCSS and relevant performance-based tests support that the 4D-NPCCSS is well-defined and appropriately standardized for use in clinical trials in NPC and provide additional support for the validity of all four domains. A 1-point difference in the score constitutes a clinically meaningful change in condition for a patient with NPC.

Because the NPCCSS did not have a pre-determined minimal clinically important difference (MCID) meaningful change in 4D-NPCCSS was discussed in interviews with caregivers/patients and clinicians. In the interviews with patients and caregivers, more than 70% of respondents stated that a 1-point change is meaningful in any of the 4D-NPCCSS domains. An even larger proportion of participants (77.8%) indicated that any slowing of disease progression would be meaningful. Therefore, a 1-point change in the 4D-NPCCSS was established as a clinically meaningful threshold.

In yet another embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the administration of the compound having a structure of Formula I results in a decrease in at least one scale score selected from SARA GAIT, SARA Finger chase, SARA Nose-finger test, SARA Fast alternating hand movements, 9-HPT, and SARA Speech disturbance.

“SARA” or “S.A.R.A.” as used herein, means Scale for the Assessment and Rating of Ataxia. SARA is a clinical scale developed by Schmitz-Hübsch et al. which assesses a range of different impairments in cerebellar ataxia (Weyer A, Abele M, Schmitz-Hubsch T, Schoch B, Frings M, Timmann D. Reliability And validity of the scale for the assessment and rating of ataxia: a study in 64 Ataxia patients. Movement Disorders 2007; 22:1633-7). The scale is made up of 8 items related to gait, stance, sitting, speech, finger-chase test, nose-finger test, fast alternating movements and heel-shin test. Once each of the 8 categories have been assessed, the total is calculated to determine the severity of ataxia. “9-HPT_ as used herein, means Nine Hole Peg Test which is a standardized assessment used to measure fine motor dexterity and hand function, often in individuals with neurological conditions. It involves placing and removing nine pegs into nine holes on a board, with the time taken to complete the task being recorded.

In yet another embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a still further embodiment, the pharmaceutically acceptable salt is citrate; alternatively, the compound of Formula I is arimoclomol citrate.

In yet another embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the compound having structure of Formula I is N-[(2R,Z)-2-hydroxy-3-(1-piperidyl)propoxy]pyridine-3-carboximidoyl chloride, 1-oxide citrate.

In yet another embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the therapeutically effective amount of the compound having the structure of Formula I is a daily dosage of about 1 to about 30 mg/kg/day; alternatively, the daily dosage is 30 mg/kg/day. In a still further embodiment, the therapeutically effective amount ranges from about 0.1 mg to about 600 mg per day; alternatively the therapeutically effective amount ranges from about 50 mg to about 500 mg per day; alternatively, from about 100 mg to about 400 mg per day. In certain embodiments, the therapeutically effective amount is selected from about 141 mg per day. About 189 mg per day, about 279 mg per day, or about 372 mg per day. In an alternative embodiment, the therapeutically effective amount is selected from about 93 mg per day, about 141 mg per day, about 189 mg per day, about 279 mg per day, or about 372 mg per day.

In yet another embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the daily dosage is administered in about 1-3 unit doses per day. In a still further embodiment, the daily dosage is administered in 3 unit doses per day.

In yet another embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the therapeutically effective amount is selected from 31 mg t.i.d, 47 mg t.i.d., 63 mg t.i.d., 93 mg t.i.d., or 124 mg t.i.d. “T.i.d,” as used herein, means three times per day.

In yet another embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is combined with soft food prior to administration; alternatively, the composition is dispersed in water prior to administration.

In yet another embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is an oral dosage formulation. In a still further embodiment, the oral dosage formulation is a solid oral dosage formulation. In certain embodiments, the solid oral dosage is selected from tablets, capsules, caplets, films, and powders and may further comprises one or more excipients.

In yet another embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the oral dosage formulation is a liquid oral dosage formulation. In a still further embodiment, the liquid oral dosage formulation comprises 3.1 mg/mL arimoclomol. In certain embodiments, the liquid oral dosage formulation further comprises a thickening agent; alternatively, the liquid oral dosage formulation is formulated for administration via a feeding tube or gastric tube.

In yet another embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is formulated for parenteral administration.

In yet another embodiment, the present disclosure provides a method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, wherein the administration of the compound having a structure of Formula I results in a decrease in at least one scale score selected from SARA GAIT, SARA Finger chase, SARA Nose-finger test, SARA Fast alternating hand movements, 9-HPT, and SARA Speech disturbance.

For patients with renal impairment, the method further comprises the steps of determining the Estimated Glomerular Filtration Rate (eGFR) of the subject/patient; and modifying the therapeutically effective amount based on whether the patient has mild to moderate renal impairment or moderate to severe renal impairment. “Moderate to severe renal impairment,” as used herein means a eGFR ranging from 15 mL/min to 50 mL/min. The therapeutically effective amount of the compound having a structure of Formula I, or a pharmaceutically acceptable salt thereof, for patients with moderate to severe renal impairment is about 1 to about 15 mg/kg/day. In one embodiment, the therapeutically effective amount is 15 mg/kg/day. In certain embodiments, the therapeutically effective amount is administered in 2 unit doses per day.

In an embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

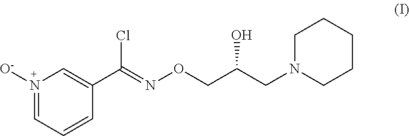

or a pharmaceutically acceptable salt thereof, wherein administering the compound having a structure of Formula I reduces or minimizes the progression of NPC disease as compared to a placebo group relative to a patient not administered a therapeutically effective amount of the compound of Formula I, or a pharmaceutically acceptable salt thereof.

“Reducing NPC disease progression,” as used herein, means any of the following: (1) slowing the rate of NPC disease progression; (2) halting NPC disease progression; or (3) reversing NPC disease progression. A non-limiting example of slowing the rate of NPC disease progression would be a patient exhibiting an overall increase in 4D-NPCCSS domain scale score that is smaller than the increase measured at a previous testing, i.e. an overall increase of one (1) point compared to an overall increase of two (2) or more points at the previous observation. A non-limiting example of halting NPC disease progression would be no overall increase in 4D-NPCCSS domain scale score that is smaller than the increase measured at a previous testing, i.e. an overall increase of zero (0) points compared to an overall increase of one (1) or more points at the previous observation. A non-limiting example of reversing NPC disease progression is an overall decrease the patient's 4D-NPCCSS domain scale score, i.e. an overall decrease of one (1) point compared to an overall increase of zero (0) or more points at the previous observation.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the NPC disease is selected from the group consisting of NPC, Type 1 (NPC-1) and NPC Type 2 (NPC2). Alternatively, the NPC disease is NPC1.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a still further embodiment, the pharmaceutically acceptable salt is citrate; alternatively, the compound of Formula I is arimoclomol citrate.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the compound having structure of formula (I) is N-[(2R,Z)-2-hydroxy-3-(1-piperidyl)propoxy]pyridine-3-carboximidoyl chloride, 1-oxide citrate.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the therapeutically effective amount of the compound having the structure of Formula I is a daily dosage of about 1 to about 30 mg/kg/day; alternatively, the daily dosage is 30 mg/kg/day. In a still further embodiment, the therapeutically effective amount ranges from about 0.1 mg to about 600 mg per day; alternatively the therapeutically effective amount ranges from about 50 mg to about 500 mg per day; alternatively, from about 100 mg to about 400 mg per day. In certain embodiments, the therapeutically effective amount is selected from about 141 mg per day. About 189 mg per day, about 279 mg per day, or about 372 mg per day. In an alternative embodiment, the therapeutically effective amount is selected from about 93 mg per day, about 141 mg per day, about 189 mg per day, about 279 mg per day, or about 372 mg per day.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the daily dosage is administered in about 1-3 unit doses per day; alternatively, the daily dosage is administered in 3 unit doses per day.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the therapeutically effective amount is selected from 47 mg t.i.d., 63 mg t.i.d., 93 mg t.i.d., or 124 mg t.i.d. “T.i.d,” as used herein, means three times per day.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is combined with soft food prior to administration; alternatively, the composition is dispersed in water prior to administration.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is an oral dosage formulation. In a further embodiment, the oral dosage formulation is a solid oral dosage formulation. In a still further embodiment, the solid oral dosage is selected from the group consisting of tablets, capsules, caplets, films, and powders. In certain embodiments, the solid oral dosage form further comprises one or more excipients.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the oral dosage formulation is a liquid oral dosage formulation. In a still further embodiment, the liquid oral dosage formulation comprises 3.1 mg/mL arimoclomol. In certain embodiments, the liquid oral dosage formulation further comprises a thickening agent; alternatively, the liquid oral dosage formulation is formulated for administration via a feeding tube or gastric tube.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is formulated for parenteral administration.

In yet another embodiment, the present disclosure provides a method for NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, wherein the administration of the compound having a structure of Formula I results in a decrease in at least one scale score selected from SARA GAIT, SARA Finger chase, SARA Nose-finger test, SARA Fast alternating hand movements, 9-HPT, and SARA Speech disturbance.

For patients with renal impairment, the method further comprises the steps of determining the Estimated Glomerular Filtration Rate (eGFR) of the subject/patient; and modifying the therapeutically effective amount based on whether the patient has mild to moderate renal impairment or moderate to severe renal impairment. “Moderate to severe renal impairment,” as used herein means a eGFR ranging from 15 mL/min to 50 mL/min. The therapeutically effective amount of the compound having a structure of Formula I, or a pharmaceutically acceptable salt thereof, for patients with moderate to severe renal impairment is about 1 to about 15 mg/kg/day. In one embodiment, the therapeutically effective amount is 15 mg/kg/day. In certain embodiments, the therapeutically effective amount is administered in 2 unit doses per day.

A method of delaying NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

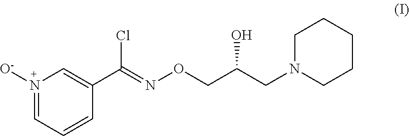

or a pharmaceutically acceptable salt thereof, wherein administering the compound having a structure of Formula I delays the progression of NPC disease as compared to a placebo group.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the NPC disease is selected from the group consisting of NPC, Type 1 (NPC-1) and NPC Type 2 (NPC2). Alternatively, the NPC disease is NPC1.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a still further embodiment, the pharmaceutically acceptable salt is citrate; alternatively, the compound of Formula I is arimoclomol citrate.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the compound having structure of formula (I) is N-[(2R,Z)-2-hydroxy-3-(1-piperidyl)propoxy]pyridine-3-carboximidoyl chloride, 1-oxide citrate.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the therapeutically effective amount of the compound having the structure of Formula I is a daily dosage of about 1 to about 30 mg/kg/day; alternatively, the daily dosage is 30 mg/kg/day. In a still further embodiment, the therapeutically effective amount ranges from about 0.1 mg to about 600 mg per day; alternatively the therapeutically effective amount ranges from about 50 mg to about 500 mg per day; alternatively, from about 100 mg to about 400 mg per day. In certain embodiments, the therapeutically effective amount is selected from about 141 mg per day. About 189 mg per day, about 279 mg per day, or about 372 mg per day. In an alternative embodiment, the therapeutically effective amount is selected from about 93 mg per day, about 141 mg per day, about 189 mg per day, about 279 mg per day, or about 372 mg per day.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the daily dosage is administered in about 1-3 unit doses per day; alternatively, the daily dosage is administered in 3 unit doses per day.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the therapeutically effective amount is selected from 47 mg t.i.d., 63 mg t.i.d., 93 mg t.i.d., or 124 mg t.i.d. “T.i.d,” as used herein, means three times per day.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is combined with soft food prior to administration; alternatively, the composition is dispersed in water prior to administration.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is an oral dosage formulation. In a further embodiment, the oral dosage formulation is a solid oral dosage formulation. In a still further embodiment, the solid oral dosage is selected from the group consisting of tablets, capsules, caplets, films, and powders. In certain embodiments, the solid oral dosage form further comprises one or more excipients.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the oral dosage formulation is a liquid oral dosage formulation. In a still further embodiment, the liquid oral dosage formulation comprises 3.1 mg/mL arimoclomol. In certain embodiments, the liquid oral dosage formulation further comprises a thickening agent; alternatively, the liquid oral dosage formulation is formulated for administration via a feeding tube or gastric tube.

In yet another embodiment, the present disclosure provides for a method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I, or a pharmaceutically acceptable salt thereof, wherein the composition is formulated for parenteral administration. For patients with renal impairment, the method further comprises the steps of determining the Estimated Glomerular Filtration Rate (eGFR) of the subject/patient; and modifying the therapeutically effective amount based on whether the patient has mild to moderate renal impairment or moderate to severe renal impairment. “Moderate to severe renal impairment,” as used herein means a eGFR ranging from 15 mL/min to 50 mL/min. The therapeutically effective amount of the compound having a structure of Formula I, or a pharmaceutically acceptable salt thereof, for patients with moderate to severe renal impairment is about 1 to about 15 mg/kg/day. In one embodiment, the therapeutically effective amount is 15 mg/kg/day. In certain embodiments, the therapeutically effective amount is administered in 2 unit doses per day.

### EXAMPLES

The presently described technology and its advantages will be better understood by reference to the following examples. These examples are provided to describe specific embodiments of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### Example 1—Effect of Arimoclomol on the Transcription Factors TFEB and TFE3

Human wild type fibroblasts and HeLa cells were incubated with various concentrations of arimoclomol (0-400 μM) in the presence of U-18666A (0.5 μM). U-18666A is an NPC1 protein inhibitor that has been shown to induce an NPC-like phenotype.

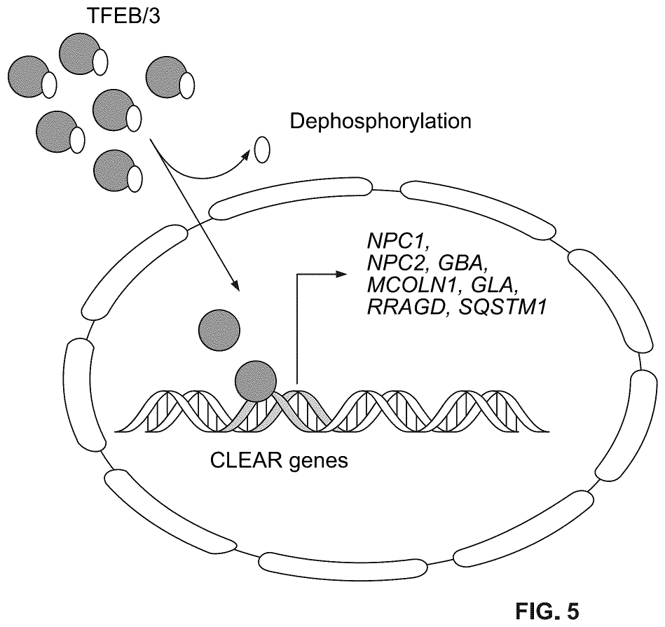

FIG. 5 shows mechanism by which arimoclomol enhances transport of TFE3 and TFEB (tested in HeLa cells only) from the cytosol to the nucleus in a dose-dependent manner. Without wishing to be bound by theory, arimoclomol enhances dephosphorylation of TFEB and TFE3, which allows for increased transport of TFEB and TFE3 through the nuclear membrane. Once inside the nucleus, the increased concentration of TFEB and TFE3 results in increased expression of the CLEAR genes. Both transcription factors, but particularly TFEB, have been identified as key components in enhancing lysosomal catabolic efficiency. An investigation of TFE3 and TFEB in healthy and NPC patient fibroblasts provided evidence that arimoclomol treatment increases the activation and translocation of these transcription factors to the nucleus and produces subsequent upregulation of the CLEAR network genes including NPC1 and HSPA1A (encoding HSP70 chaperone/heat shock protein). In addition, it has been extensively reported that TFEB governs the expression of genes critical for the autophagy process including autophagy initiation, autophagosome membrane elongation, substrate capture, and autophagosomes trafficking and fusion with lysosomes (Palmieri et al., 2011; Settembre et al., 2011; Martina et al., 2014; Sardiello, 2016). Therefore, activation of TFE3/TFEB would be expected to initiate a marked increase in autophagy to support degradation of undesirable intracellular components.

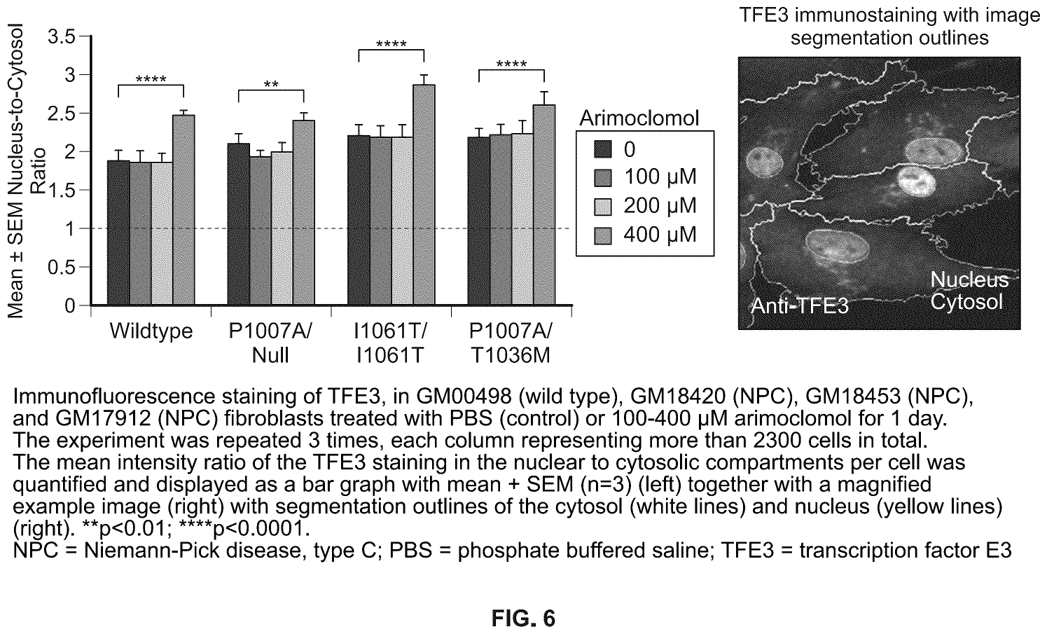

FIG. 6 shows the mean nuclear-to-cytosol ratio of TFE3 in healthy human fibroblasts and NPC human fibroblasts. Exposure to 100 μM and 200 μM concentrations of arimoclomol generally led to small changes in the nuclear-to-cytosol ratio. Exposure to 400 μM concentrations of arimoclomol led to significant increases in the nuclear-to-cytosol ratio. Immunofluorescence staining of TFE3, in GM00498 (wild type), GM18420 (NPC), GM18453 (NPC), and GM17912 (NPC) fibroblasts treated with PBS (control) or 100-400 μM arimoclomol for 1 day. The experiment was repeated 3 times, each column representing more than 2300 cells in total. The mean intensity ratio of the TFE3 staining in the nuclear to cytosolic compartments per cell was quantified and displayed as a bar graph with mean+SEM (n=3) (left) together with a magnified example image (right) with segmentation outlines of the cytosol (white lines) and nucleus (yellow lines) (right). **p<0.01; ****p<0.0001.

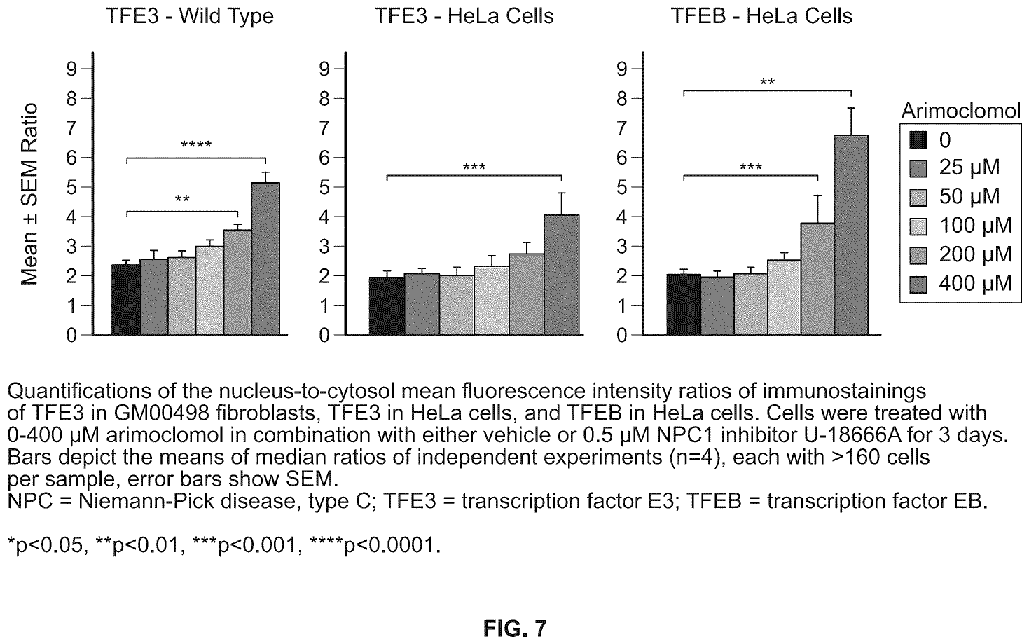

FIG. 7 shows the mean nucleus-to-cytosol ration of TFE3 and TFEB in healthy human fibroblasts compared to NPC human fibroblasts when both the healthy and NPC fibroblasts were exposed to the NPC1 inhibitor U-1866A. Generally, exposure to arimoclomol in the presence of the NPC1 inhibitor led to an increase in the nucleus-to-cytosol ratio of both TFEB and TFE3 in healthy and NPC fibroblasts. Exposure to concentrations of greater than 100 μM of arimoclomol resulted in significant increases in the nucleus-to-cytosol TFEB and TFE3 ratios.

Taken together, FIG. 6 and FIG. 7 demonstrate a key component of both the NPC1-dependent and the NPC1-independent pathway by showing how arimoclomol facilitates the mobilization of transcription factors TFE3/TFEB, paving the way for the upregulation of CLEAR genes, including NPC1.

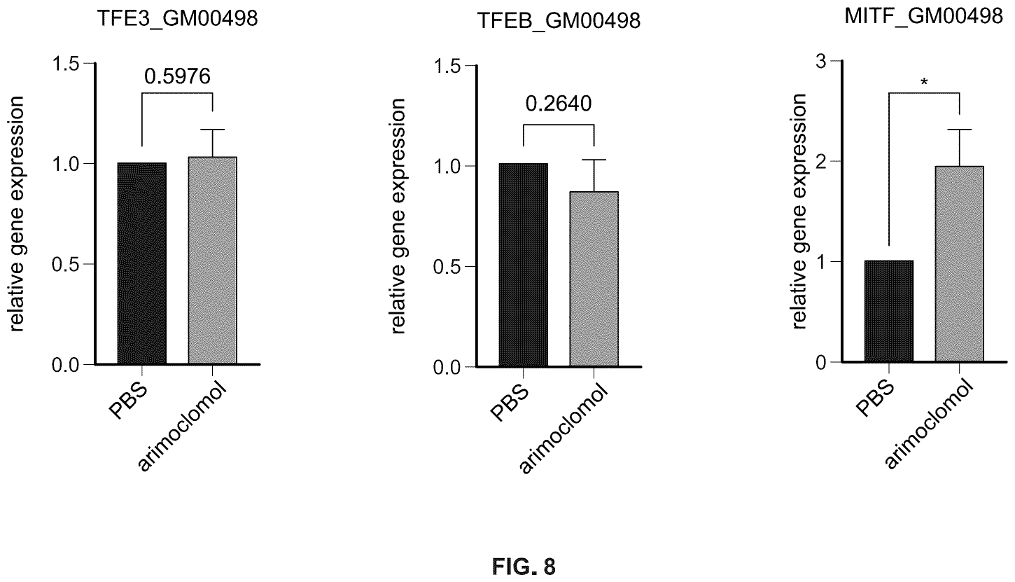

FIG. 8 shows the effect of arimoclomol on the expression of TFEB and TFE3. Expression of MITF is increased 2-fold but neither TFE3 or TFEB expression show increased expression following arimoclomol treatment. “MITF,” as used herein, means melanocyte inducing transcription factor. Increasing expression of MITF indicates but not TFEB and/or TFE3 indicates that arimoclomol increases the amount of TFEB and TFE3 translocated to the nucleus by activating the dephosphorylation reaction that allows TFEB and TFE3 to enter the nucleus, as opposed to increasing TFEB and TFE3 translocation simply by increasing the amount of TFEB and TFE3 available for dephosphorylation. Without wishing to be bound by theory, it is believed that TFEB and TFE3 expression are not enhanced with arimoclomol in this cell line, because they are regulated at the protein level instead of at the transcription level.

### Example 2—Effect of Arimoclomol on the Expression of CLEAR Network Genes

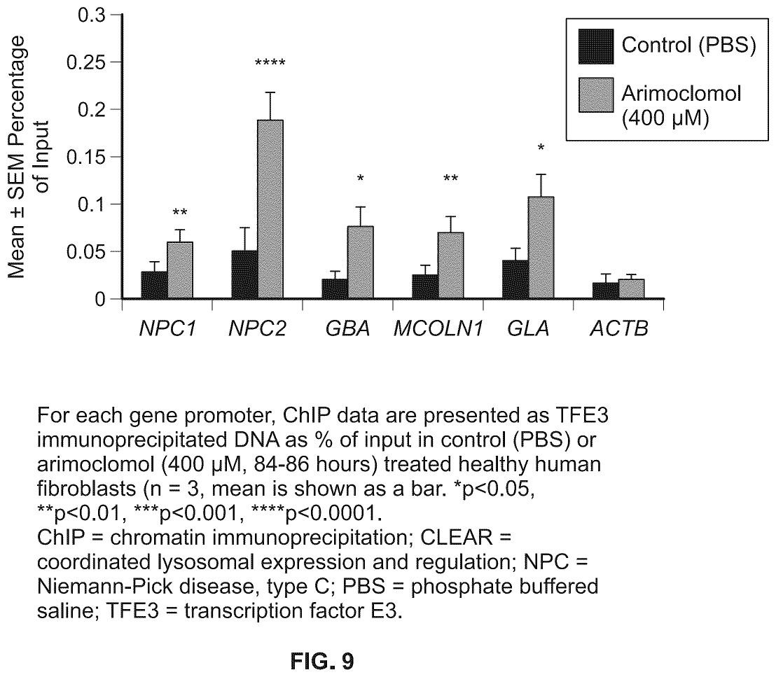

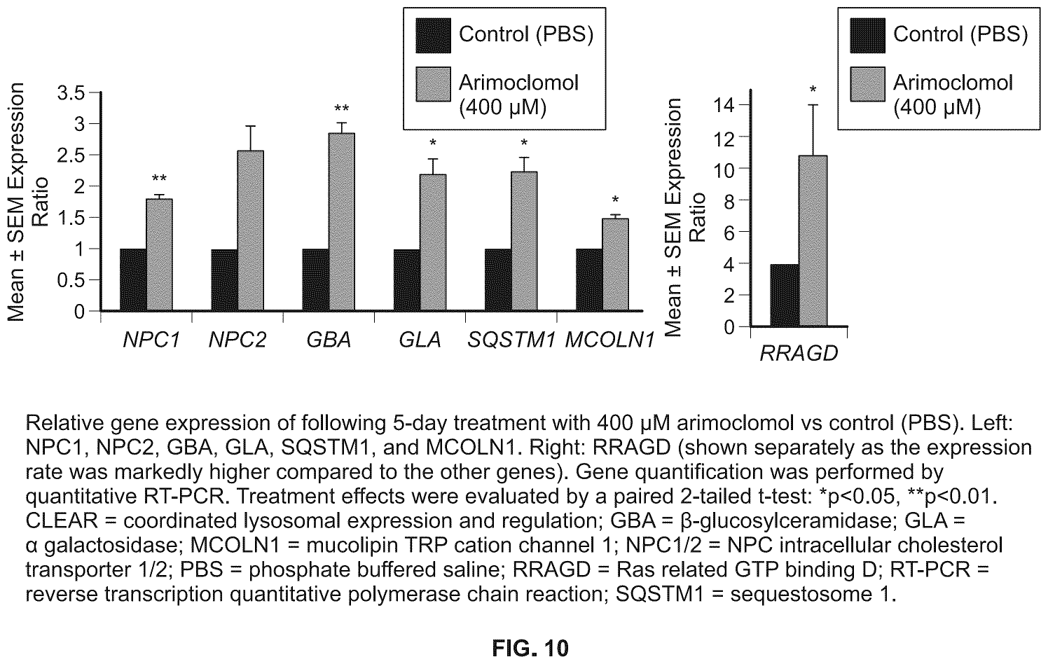

FIG. 9 and FIG. 10 show that transcriptional upregulation of all 7 tested CLEAR genes was observed with arimoclomol (400 μM). FIG. 9 shows that treatment with arimoclomol (400 M) significantly enhanced the binding of TFE3 to the CLEAR promoter elements of NPC1, NPC2, GBA, MCOLN, and GLA in human wild type fibroblasts. FIG. 10 shows that additional CLEAR genes related to lysosomal function (NPC1, NPC2, GBA, GLA, MCOLN1, RRAGD, SQSTM1) were investigated to determine whether expression of these genes was upregulated by arimoclomol in healthy human fibroblasts. The mean expression levels ranged from 1.48-fold to 10.86-fold higher vs control with most genes being expressed at approximately 2-3-fold higher rates. Mean NPC1 expression was 1.82-fold of control. These results indicate that arimoclomol stimulates a CLEAR response for various genes. Each protein expression level is normalized to the untreated expression concentration. NPC1 and NPC2 expression levels roughly tracked with results for TFEB and TFE3 binding studies, and an over all 2-3 fold increase in protein expression levels.

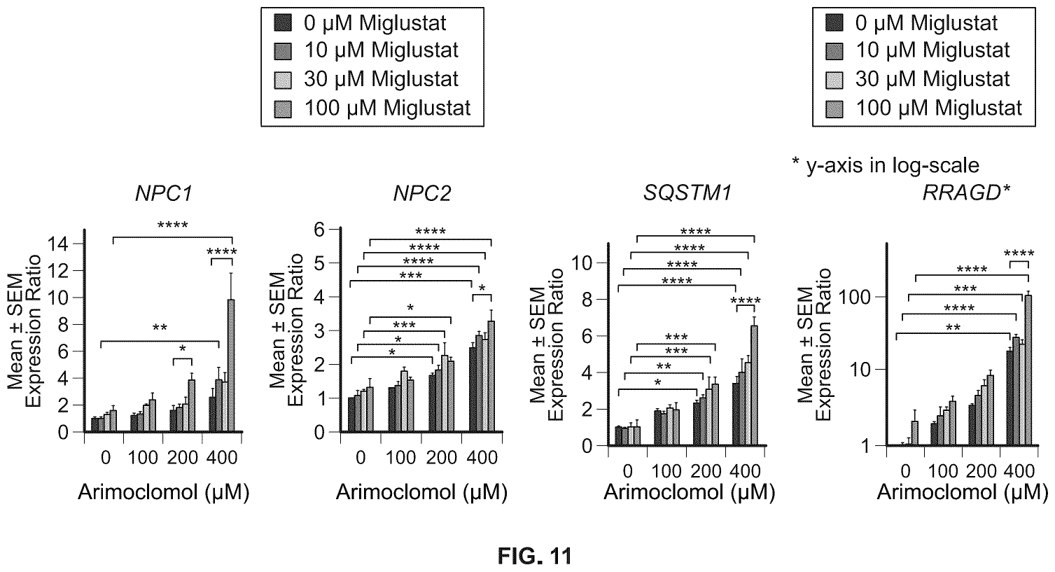

FIG. 11 shows the increased levels of CLEAR gene proteins when exposed to both arimoclomol and miglustat. The expression rates of all 7 CLEAR genes as well as HSPA1A increased in a dose-dependent manner with arimoclomol alone and miglustat alone. When either treatment was dosed alone at 100 μM, the fold-increase in expression with either compound was comparable for NPC1, NPC2, and RRAGD. At 100 μM, expression rates were higher with miglustat for GBA, GLA, MCOLN1 (encoding TRPML1, an iron channel located in endosomal/lysosomal membranes) and HSPA1A, and higher with arimoclomol for SQSTM1 (encoding sequestosome-1, an autophagosome cargo protein). At concentrations >100 M (without miglustat), arimoclomol continued to accelerate expression levels of all 7 genes reaching statistical significance for NPC2, RRAGD, and SQSTM1, and HSPA1A at 400 PM. Notably, a complementary effect of arimoclomol and miglustat was observed for the 7 CLEAR genes under nearly all conditions. This complementary effect reached statistical significance for NPC1, GLA, RRAGD, and SQSTM1 (at 400 and 100 μM for arimoclomol and miglustat, respectively), but not for NPC2 and GBA when compared to arimoclomol alone. A statistically significant increase of MCOLN1 expression over just arimoclomol was found at 200/100 μM arimoclomol/miglustat but not at 400/100 M.

A complimentary effect of arimoclomol and miglustat was observed for the 7 CLEAR genes under nearly all conditions. This complimentary effect reached statistical significance for NPC1, GLA, RRAGD, and SQSTM1 (at 400 and 100 μM for arimoclomol and miglustat, respectively), but not for NPC2 and GBA when compared to arimoclomol alone. A statistically significant increase of MCOLN1 expression over just arimoclomol was found at 200/100 M arimoclomol/miglustat but not at 400/100 M.

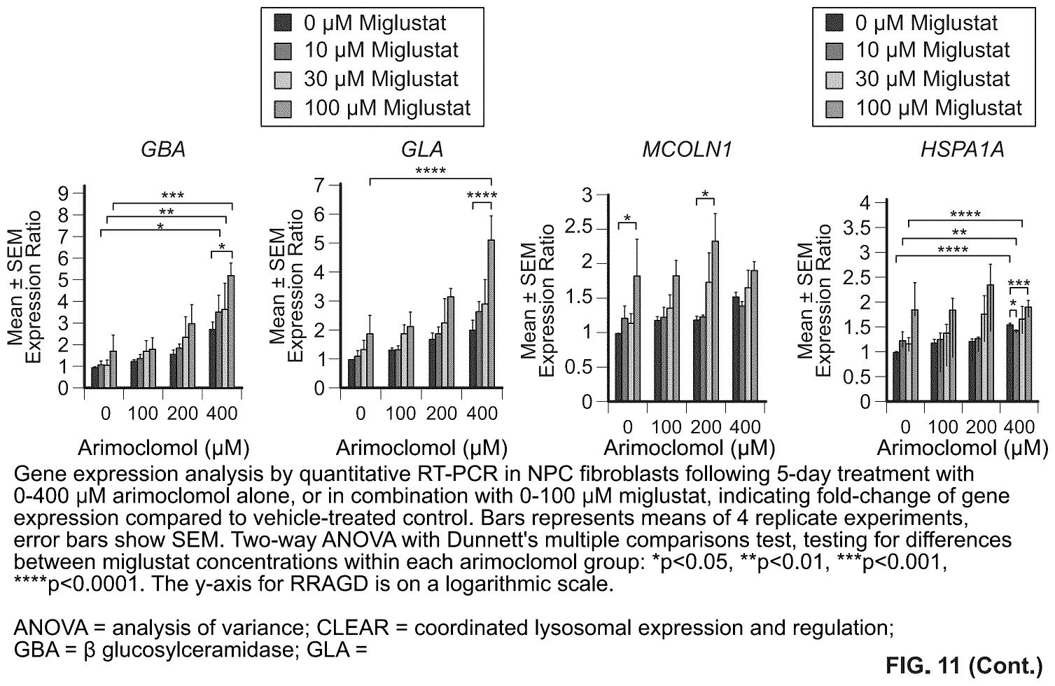

FIG. 12 shows the expression of all 3 test genes was enhanced by arimoclomol after 2 or 5 days of incubation in a generally dose-dependent manner. After 2 and 5 days of treatment, statistically significant increases over control (PBS) were reported at 400 μM arimoclomol for all 3 genes in both cell lines except for GBA in fibroblasts with the endoplasmic reticulum mutations after 5 days. A statistically significant difference relative to control was also found for HSPA1A at 200 μM arimoclomol in I1061T/I1061T fibroblasts after 2 days.

### Example 3—Effect of Arimoclomol on Gene Expression of NPC1, GBA, and HSPA1A

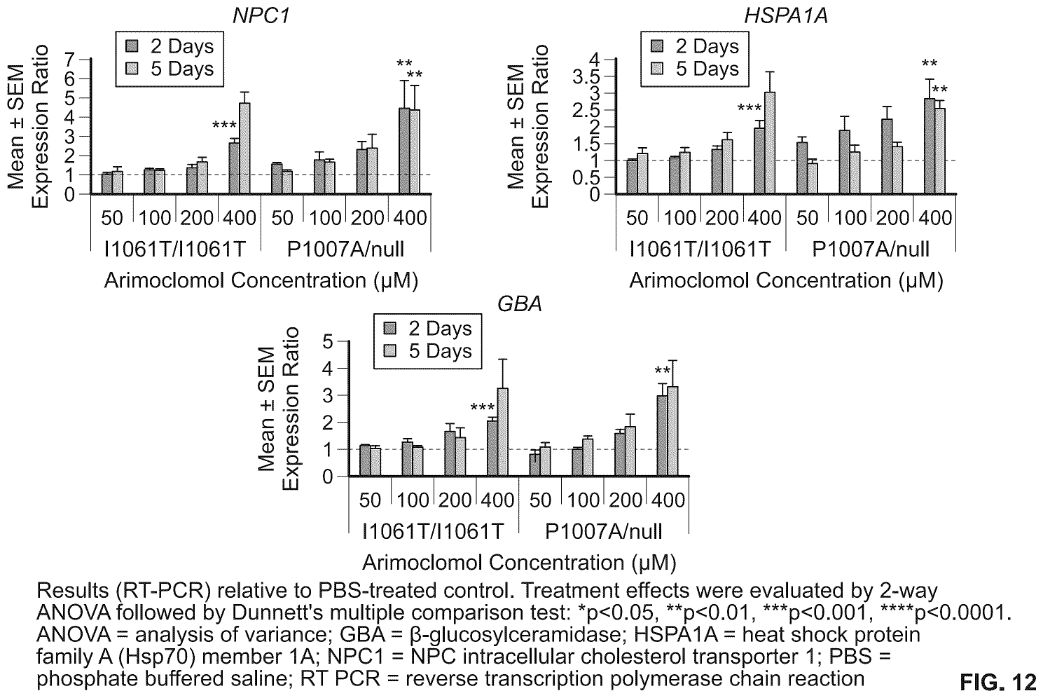

FIG. 13 shows the overall levels of NPC1 protein expression by human fibroblasts with different mutations in the NPC1 gene. Overall NPC1 protein expression depends on the type of mutation.

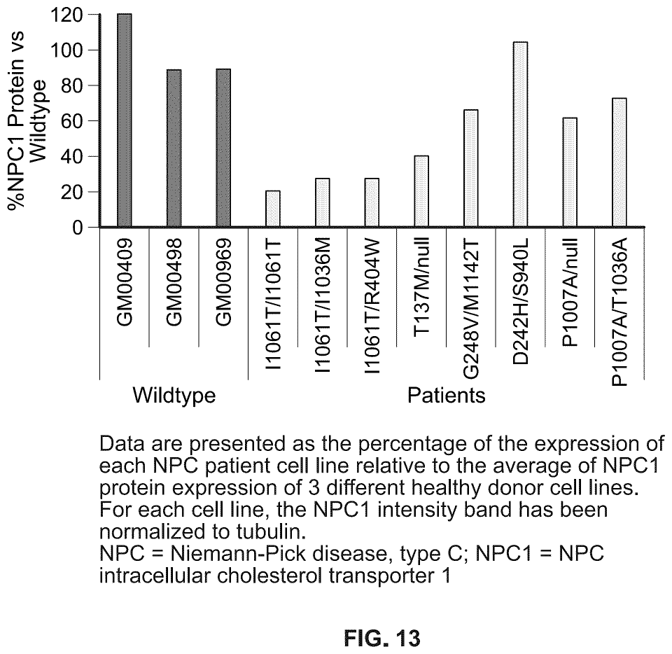

FIG. 14 shows the effects of arimoclomol treatment on fibroblasts with different NPC1 gene mutations. The NPC donor fibroblasts in this study included 3 common types of mutations that can produce functional NPC1 protein if folded properly and trafficked to the target organelles (e.g., late endosomes and lysosomes) (Shammas et al., 2019). The first type comprises ER missense mutations (e.g., the most common I1061T mutation). Mutant proteins of this type are blocked in the ER due to misfolding and early degradation. The second type consists of proteins that can reach the target late endosomes, but their transport occurs at lower rates compared to the wild type protein (“delayed”). The third type exhibits trafficking patterns similar to wild type (“WT-like”) and typically results in milder phenotypes with adult-onset of disease symptoms (e.g., P1007A mutation). At baseline, all genotypes, except for one (GM18390: D242H/S940L) showed lower NPC1 protein concentrations (21-73% of wild type) than the wild type. In the second part of the study, the various mutant fibroblasts were incubated with arimoclomol (0-400 μM). The results showed a mostly dose-dependent increase in NPC1 protein concentrations in all genotypes. The most pronounced effects were reported in the I1061T/I1061T (endoplasmic reticulum/endoplasmic reticulum) genotype followed by T137M/null (other/null), P1007A/T1063M (WT-like/delayed), and P1007A/null (WT-like/null). While arimoclomol increased NPC1 protein concentrations at nearly all concentrations in all genotypes in a generally dose-dependent fashion, the largest changes were found at 200 and 400 μM.

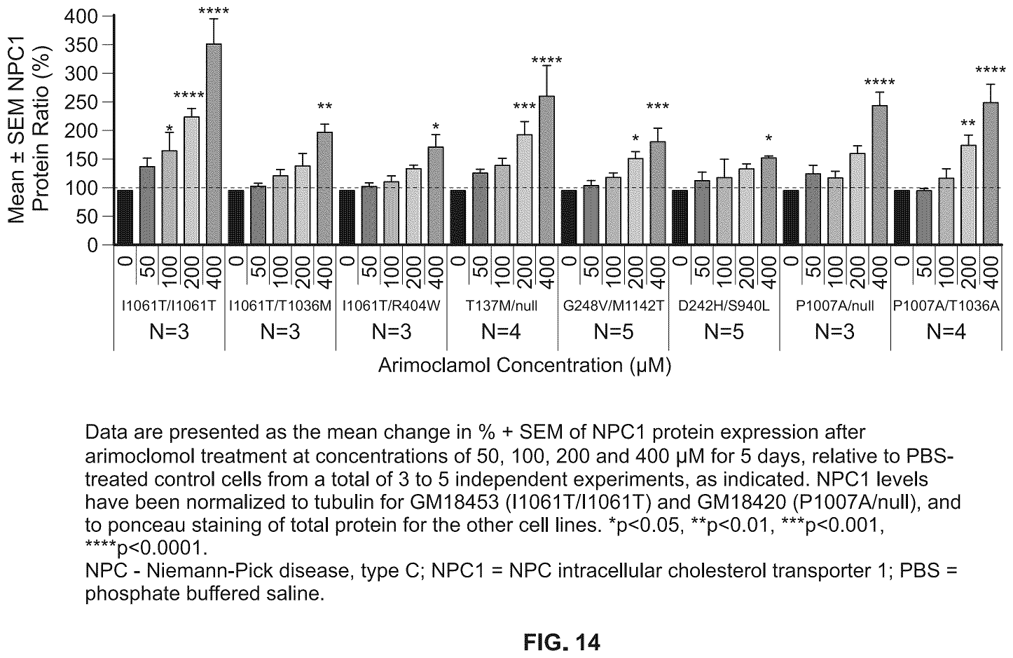

FIG. 15 shows that arimoclomol increased levels of folded and mature NPC1 mutant protein and not just overall cellular levels of NPC1 protein. Depending on the specific genotype and the functionality of the resulting mutant protein, this greater availability of trafficking competent NPC1 protein that can reach the late endosomal/lysosomal membranes results in various degrees of improved clearance of unesterified cholesterol.

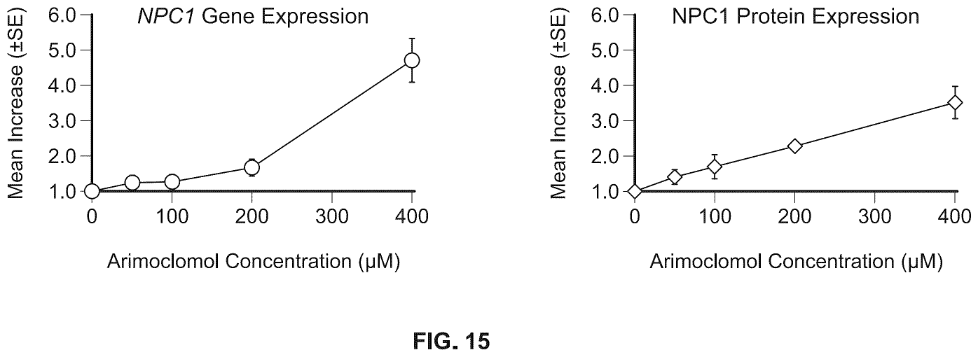

FIG. 16 shows the results for EndoH resistance assays. “EndoH resistance assay,” as used herein, means an assay technique used to study protein trafficking and glycosylation patterns within cells by cleaving specific types of N-linked glycans attached to proteins. To further the understanding of the possible clinical meaning of the arimoclomol induced increases in cellular NPC1 protein concentrations, arimoclomol was evaluated in Endo H assays (at 400 μM for 5 days) with fibroblasts of 2 different genotypes containing alleles of the most common mutations (endoplasmic reticulum missense I1061T and WT-like P1007A). Digestion of the glycan-protein complex (Endo H sensitive) indicates immature protein, while resistance to Endo H degradation implies maturation, escape from the endoplasmic reticulum, and transport through the cis-Golgi.

Expectedly, the increase of ˜1.74-fold in Endo H resistance with arimoclomol vs untreated control was higher in the genotype with the homozygous I1061T mutations (impaired endoplasmic reticulum transport) as the trafficking of NPC1P1007A protein is already comparable to the wild type and thus has a lower potential for improved transport.

Overall, the data indicate that arimoclomol does not only upregulate expression of certain CLEAR genes and specifically NPC1 at the transcriptional level, but also that this overexpression results in amplification of NPC1 protein levels and more successful NPC1 processing. This allows more protein to pass through the Golgi and reach the target late endosomes. Increased biosynthesis would also be expected for other proteins encoded by CLEAR genes due to the upregulated expression by arimoclomol.

Without wishing to be bound by theory, enhanced trafficking of NPC1 protein could be that the increased number of synthesized protein molecules improves the odds of proper folding and escape from the ER before degradation. A second or additional explanation could be that the upregulation of HSPA1A expression by arimoclomol improves the overall chaperone function of heat shock protein 70 (Hsp70) resulting in more efficient and successful protein folding (Study DOC-2201240047) (Kieran et al., 2004; Kalmar et al., 2008; Neef et al., 2011).

### Example 4—Effects of Arimoclomol on Concentrations, Maturation, and Trafficking of NPC1 Protein

To verify that arimoclomol can raise the rate of NPC1 biosynthesis and improve its maturation Study DOC-2004170028 was conducted to assess NPC1 protein levels in wild type and NPC patient fibroblasts. Additionally, sensitivity of glycosylated NPC1 protein to endoglycosidase H (Endo H) hydrolysis was explored to determine the mobility and localization of the protein complex. Nascent membrane proteins like NPC1 are glycosylated with mannose-rich N-glycans as they enter the lumen of the endoplasmic reticulum (Freeze and Kranz, 2010). These immature glycans can be cleaved by Endo H. However, as the proteins mature during migration to the Golgi, the glycan chains are heavily processed and become resistant to Endo H hydrolysis (Gelsthorpe et al., 2008).

The NPC donor fibroblasts in this study included 3 common types of mutations that can produce functional NPC1 protein if folded properly and trafficked to the target organelles (e.g., late endosomes and lysosomes) (Shammas et al., 2019). The first type comprises endoplasmic reticulum missense mutations (e.g., the most common I1061T mutation). Mutant proteins of this type are blocked in the endoplasmic reticulum due to misfolding and early degradation. The second type consists of mutants that can reach the target late endosomes, but their transport occurs at lower rates compared to the wild type protein (“delayed”). The third type exhibits trafficking patterns similar to wild type (“WT-like”) and typically results in milder phenotypes with adult-onset of disease symptoms (e.g., P1007A mutation).

### Example 5—Effects of Arimoclomol of the Clearance of Unesterified Cholesterol in Human NPC Fibroblasts

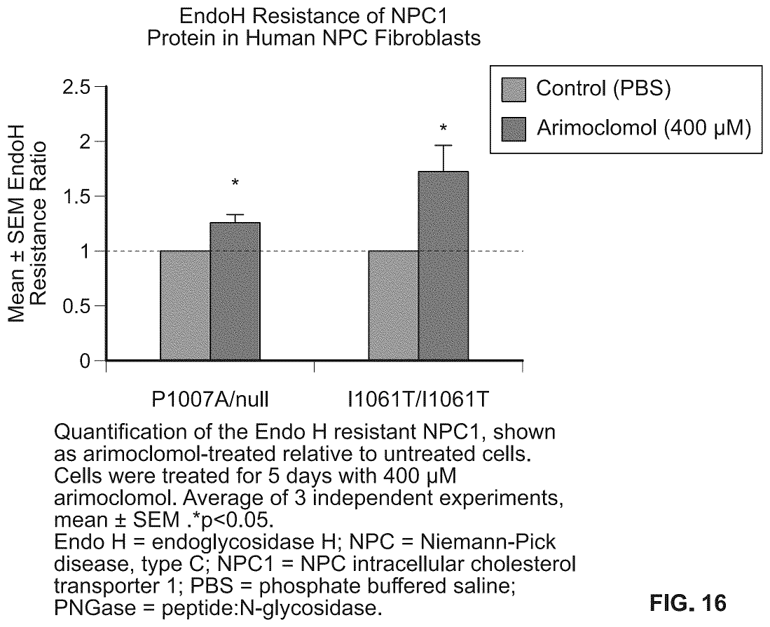

FIG. 17 shows that arimoclomol treatment increases the expression of NPC1 protein which in turn increases the cholesterol clearance from lysosomes. shown that increasing the expression of NPC1 and other CLEAR genes with arimoclomol can activate or accelerate cholesterol clearance from lysosomal compartments. In addition, to investigate the effects of combined treatment with arimoclomol (0-200 μM) and miglustat (0-100 μM) on the cholesterol clearance in NPC fibroblasts. After incubation for 7 days, a dose-dependent reduction in unesterified cholesterol in the lysosomal compartment was observed with arimoclomol alone compared to vehicle control although the percent difference in Filipin staining intensities between the lowest (50 μM) and the highest (200 μM) dose was relatively small (83% vs 79% of untreated).

The experiments were repeated with an extended treatment period of 14 days indicated that maximum effect with arimoclomol alone took 14-21 days in human fibroblast). As expected, the unesterified cholesterol content in the lysosomal compartment continued to decrease beyond 7 days of treatment relative to control. Overall, the pattern between arimoclomol/miglustat concentrations and Filipin staining intensities were comparable after 7 and 14 days of treatment. However, the apparent reductions in unesterified cholesterol were markedly larger after 14 days vs 7 days at most doses. Stain intensities were comparable at 50 and 100 μM at any given miglustat dose, but a considerable drop was found between 100 and 200 μM arimoclomol. Lysosomal cholesterol content decreased in a dose-dependent manner with miglustat from 10-100 μM. Statistically significantly reduced Filipin staining intensity was found following arimoclomol treatment at 200 μM at all prespecified time points (at 7, 14, 21, and 28 days) and at 21 and 28 days after treatment with 100 μM arimoclomol. Additionally, unesterified cholesterol levels decreased numerically between Day 7 and 14 and reached stable levels between Day 21 and 28. These data demonstrate that the arimoclomol enhanced transcription processes can indeed rescue the NPC phenotypes evaluated in this study.

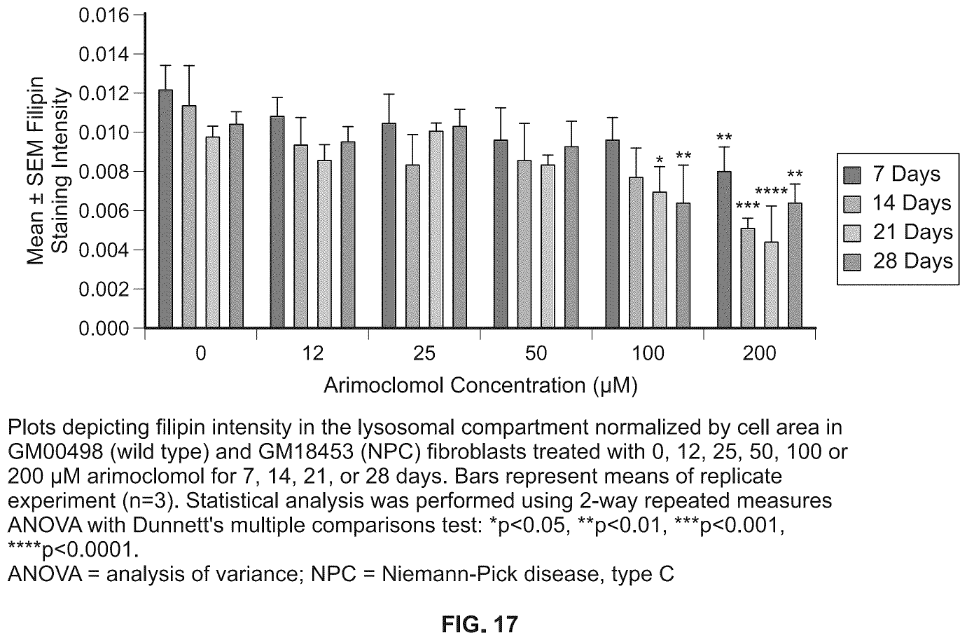

FIG. 18 shows the results of a 14-day study examining the effects of arimoclomol treatment in combination with miglustat treatment. Stain intensities were comparable at 50 and 100 μM at any given miglustat dose, but a considerable reduction was found by increasing the arimoclomol concentration from 100 to 200 μM. Lysosomal cholesterol content decreased in a dose-dependent manner with miglustat from 10-100 μM. Combining arimoclomol and miglustat resulted in a substantial complimentary effect. At 200 μM/100 μM arimoclomol/miglustat for 14 days, a reduction in Filipin staining intensity of nearly 80% was observed compared to vehicle control. Notably, treatment for 14 days with lower dose combinations of both compounds, e.g., 50 μM/30 μM arimoclomol/miglustat, already reduced lipid burden by approximately 50%.

These data are consistent with reports that miglustat can decrease GSL accumulation with downstream reduction of cholesterol found in NPC fibroblasts Therefore, it appears that arimoclomol and miglustat can act on independent pathways to provide complimentary effects with respect to reducing cholesterol burden in the lysosomal compartment of NPC patients. This complimentary effect of arimoclomol and miglustat was also observed in the rearing behavior and survival of Npc1^{-/-} mutant (discussed below), and in the slower progression rate (as measured by change in 4D-NPCCSS from baseline) in NPC patients receiving concomitant arimoclomol and miglustat when compared to the FAS.

### Example 6—NPC Mouse Models

Two different NPC mouse models, NPC null mutant mice (referred to as Npc1^{-/-} or Npc^{nih}) and homozygous NpC1^{nmf164} (or Npc1^{nmf/nmf}) missense mutant mice, were studied to potentially capture effects of arimoclomol resulting from pathways associated with NPC1 protein biosynthesis and trafficking (NpC1^{nmf164} mice), and from processes that may engender treatment effects independent of NPC1 protein related pathways (Npc1^{-/-} mice).

Npc1^{-/-} mice have 2 functional null mutations and therefore are unable to produce functional Npc1 mRNA or NPC1 protein. Consequently, any observed therapeutic effects of arimoclomol in Npc1^{-/-} mice would be due to NPC1-independent pathways. Based on the in vitro data, these key independent pathways likely include activation of the transcription factors TFEB and TFE3, their enhanced binding to relevant gene promoters, and the upregulation of CLEAR gene expression (other than NPC1). It is known that increased activation of these genes can enhance autophagosome biogenesis, autophagosome-lysosome fusion, lysosome biogenesis, and lysosomal exocytosis (Sardiello et al., 2009; Palmieri et al., 2011; Settembre et al., 2011; Martina et al., 2014; Yang and Wang, 2021). These processes may result in higher lysosomal efficiency and autophagy flux to improve overall cell health. Increased biogenesis of new lysosomes that are not yet overburdened by free cholesterol may restore some lysosomal function to facilitate clearance of cellular debris and improve cell fitness.

Npc1^{-/-} mice have an acute clinical course with an onset of disease symptoms typically around 6 weeks of age. A clear differentiation between wild type mice and NPC mice can be observed at approximately 7-8 weeks of age followed by death in Npc1^{-/-} mice at approximately 11 weeks of age.

The Npc1^{nmf164} mouse model, also referred to as the Maue NPC mouse model (Maue et al., 2012), is a point mutation model of NPC with a single base pair change (A to G) in codon 1005 of NPC1 resulting in relatively normal levels of NPC1 mRNA but substantially reduced levels of functioning NPC1^{D1005G} protein. The NPC^{1D1005G} variant does not appear to be prone to protein misfolding and ER blockage like the human NPC1^{I1061T} variant but shares more similarities with the human NPC1^{P1007A} allele that exhibits a wild type-like trafficking pattern. Since Npc1^{nmf14} mice can still produce some NPC1 protein, they may represent a more relevant mouse model than Npc1^{-/-} mice with double functional null mutations that are considerably less common in humans. However, treatment effects in Npc1^{-/-} mice would indicate that arimoclomol can act via NPC1 independent processes that benefit all patients.

In the Npc1nmf164 mouse, onset of symptoms is typically seen at approximately 9 weeks of age with a clear differentiation from wild type mice at approximately 11-13 weeks of age.

Mortality of Npc1nmf164 mice is typically reported at 16 weeks of age.

### Example 7—Comparison of Animal Doses with Clinical Doses

The PD studies in NPC mice were conducted at arimoclomol citrate doses of 1 to 500 mg/kg/day (corresponding to 0.62-310 mg/kg/day arimoclomol base). Treatment effects of arimoclomol in mutant mice were observed from 10 mg/kg/day to the highest tested dose of 500 mg/kg/day. However, consistent effectiveness was generally found at doses ≥30 mg/kg/day. Since exposure data were not collected in these PD studies, this section discusses the clinical meaningfulness of the effective dosages found in animals based on exposure data from pharmacokinetic (PK) studies of arimoclomol in mice and humans.

Arimoclomol exposure was determined in a single and multiple dose study in healthy young men receiving oral doses of 400 mg t.i.d. arimoclomol citrate (248 mg t.i.d. arimoclomol base). Mean total exposure over the first 8 hours post dose (AUC₀₋₈ₕ) was 7315 h×ng/mL at steady state. The recommended clinical dose of arimoclomol in adult NPC patients (i.e., body weight >55 kg) is 124 mg t.i.d. arimoclomol base resulting in an estimated AUC₀₋₈ₕ of 3657 ((h×ng)/mL). This also represents the approximate target exposure across all patients (recommended doses and body weight ranges in the proposed label were selected to provide comparable exposure in all patients).

In a PK study in C57Bl/6J mice receiving a single oral dose of 10 mg/kg arimoclomol citrate (6.2 mg/kg/day arimoclomol base), arimoclomol exposure as measured by AUC0-8h was 204 ((h×ng)/mL). Exposures for all doses administered in the mouse PD studies were estimated based on those data.

### Example 8—Comparison of Arimoclomol Exposure in Mice and Humans

The respective exposures to arimoclomol at all doses administered in NPC mice were then compared to the target human clinical exposure (˜3657 ((h×ng)/mL)). The resulting relative exposures in mice expressed as percentage of human target exposure are shown in Table 1:

TABLE 1
Estimated Muman-to-Animal Exposure Ratios
in Pharmacodynamic Mouse Studies.

| Mouse Dose | Relative Exposure |
|---|---|
| (mg/kg/day arimoclomol citrate) | Mouse vs Human |
| 1 | 1% |
| 3 | 2% |
| 10 | 6% |
| 30 | 17% |
| 50 | 28% |
| 100 | 56% |
| 300 | 167% |
| 500 | 279% |

### Example 9—Effects of Arimoclomol on the Survival of NPC1-Independent and NPC1-Dependent Mice

Survival was assessed in 4 of the 6 mouse PD studies: 3 in the Npc1^{-/-} (NPC1-independent) and 1 in the Npc1^{nmf164} (NPC1-dependent) mouse model. A modest but consistent positive effect on mean survival was observed with arimoclomol treatment in the dose range of 30-300 mg/kg/day in all studies. These results are summarized in Table 2.

TABLE 2
Additional Survival Time of NPC Mice Treated with Arimoclomol vs Untreated Controls.

| | | | | Additional survival | |
|---|---|---|---|---|---|
| Mouse | | Arimoclomol dose | Mean survival | vs untreated | |
| model | Study | (mg/kg/day) | (days) | Days | Percent |
| Npc1^{-/-} | CRO-1202290013 | 30 | 86.5 | 8.5 | 11 |
| | | 300 | 83.5 | 5.5 | 7.1 |
| | CRO-1402050053 | 1 | 74.9 | 2.6 | 3.6 |
| | | 3 | 71.8 | −0.5 | −0.69 |
| | | 10 | 71.8 | −0.5 | −0.69 |
| | | 30 | 74.5 | 2.3 | 3.1 |
| | | 10 b.i.d. (gavage) | 75.5 | 0.5 | 0.67 |
| | | 30/10 b.i.d. (gavage)^{b} | 75.6 | 2.3 | 3.1 |
| | DOC-2110260043 | 30 Arimoclomol | 89.0 | 5.6 | 6.7 |
| | | 30 + 600 Arimoclomol + | 141 | 57.6 | 69 |
| | | Miglustat | | | |
| Npc1^{nmf164} | DOC-2201170045 | 10 | 119 | 1.7 | 1.4 |
| | | 50 | 120 | 2.9 | 2.5 |
| | | 100 | 128 | 11.0 | 9.4 |
| | | 500 | 118 | 1.2 | 1.0 |

While the increases in mean survival age of arimoclomol-treated Npc1^{-/-} mice compared to untreated animals may appear small (+3-11%), simply looking at the group means does not provide the whole picture. Although mean survival rates are valuable, they can sometimes prompt inaccurate group inferences when being evaluated without the context of the individual survival distribution (especially at relatively low N values). To provide a better understanding of the overall data, the survival rates at various cut-off dates were compared across studies between untreated animals and animals treated at the same dose of arimoclomol (30 mg/kg/day in drinking water or approximately equivalent dose of 10 mg/kg/day b.i.d. via oral gavage).

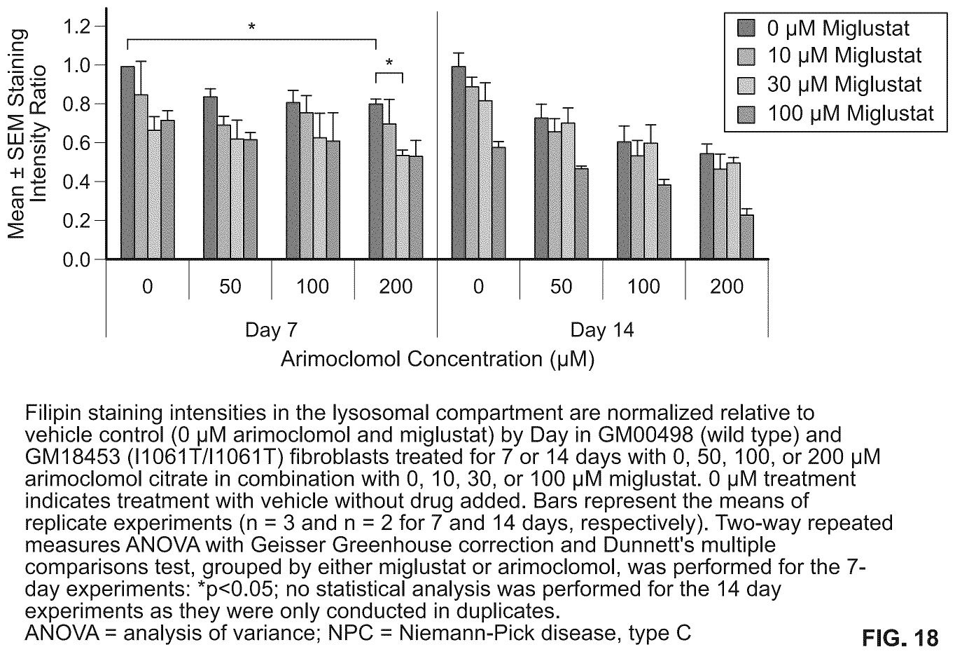

FIG. 19 shows that when treated with arimoclomol, NPC-dependent (Npc1^{nmf164}) mice demonstrated levels of mature and properly folded NPC1 protein that were comparable to levels measured in healthy, wild-type mice. In addition, the mean concentration of NPC1 protein in treated mice was approximately 50% greater than that found in untreated mice.

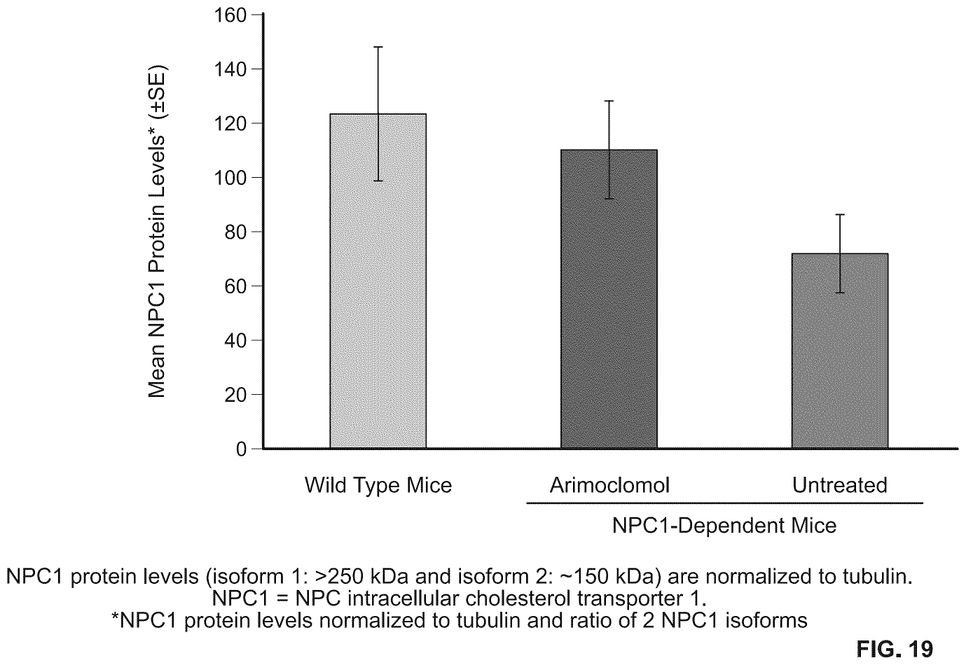

FIG. 20 shows survival of Npc1^{-/-} female mice treated with 30 mg/kg/day arimoclomol versus a control group. The pooled data across the 3 studies showed that approximately 16%, 24%, and 20% more animals treated with arimoclomol (at 30 mg/kg/day or 10 mg/kg b.i.d.) survived past 75, 80, and 85 days, respectively, when compared to the untreated animals. Overall, these data support a lengthening of survival in Npc1^{-/-} mice treated with arimoclomol. These results are summarized in Table 3.

TABLE 3
Survival Rates in Npc1^{nmf164} Mutant Mice Treated with
Arimoclomol (10-500 mg/kg/day) or Vehicle Control

| | Number of Surviving Animals (n/N [%]) | | | | | | | | |
|---|---|---|---|---|---|---|---|---|---|
| | Dose (mg/kg/day) | | | | | | | | |
| Survival Cut-off | 0 | | 10 | | 50 | 100 | | 500 | |
| >16 weeks | 5/6 | (83%) | 6/6 | (100%) | 6/7 (86%) | 6/6 | (100%) | 5/6 | (83%) |
| >17 weeks | 2/6 | (33%) | 2/6 | (33%) | 4/7 (57%) | 5/6 | (83%) | 2/6 | (33%) |
| >18 weeks | 0/6 | (0%) | 0/6 | (0%) | 1/7 (14%) | 3/6 | (50%) | 2/6 | (33%) |

| Median | (weeks) | 16.7 | 16.9 | 17.3 | 18.3** | 16.7 | |
|---|---|---|---|---|---|---|---|
| Survival | (days) | 116.8 | 118.5 | 119.7 | 127.8 | 118.0 | |
| Maximum | (weeks) | 17.7 | 17.6 | 18.1 | 19.6 | 18.3 | (n = 2) |

| | | | | | | |
|---|---|---|---|---|---|---|
| survival | (days) | 124 | 123 | 127 | 137 | 128 |

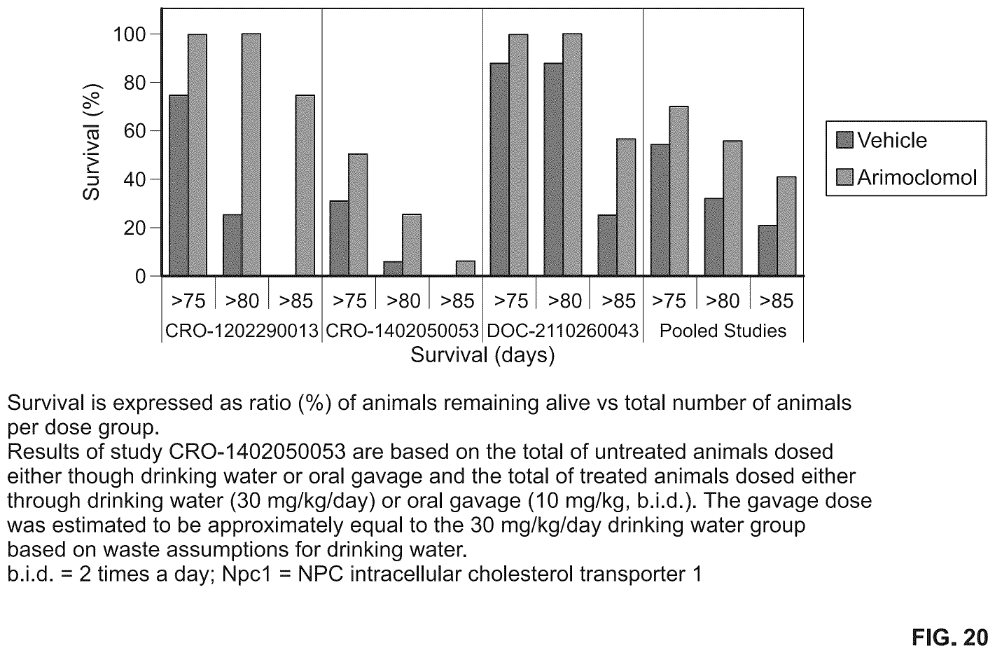

Overall, the results showed a survival benefit in animals treated with arimoclomol. At 100 mg/kg/day arimoclomol, the median survival increased by more than 11 days or approximately 10% of the total lifespan when compared to the untreated group. It was also found that Npc1^{nmf164} mutant mice lived longer compared to Npc1^{-/-} mice as expected. These results are also shown in FIG. 21. A higher percentage of animals in the 50 and 100 mg/kg/day arimoclomol group survived beyond 16, 17, and 18 weeks compared to the untreated group. At 500 mg/kg/day arimoclomol, 2 out of 6 (33%) Npc1^{nmf164} mice survived past 18 weeks, while no animal in the untreated group did. These results are supported by Filipin staining data summarized in Table 4.

TABLE 4
Filipin Staining Intensities in the Lysosomal Compartment of
Human NPC Fibroblasts as Percent of Vehicle Control After
Treatment with Arimoclomol and Miglustat for 7 and 14 days

| | Day 7 | | | | Day 14 | | | |
|---|---|---|---|---|---|---|---|---|
| Arimoclomol | | Miglustat Concentration (μM) | | | | | | |
| Concentration | 0 | 10 | 30 | 100 | 0 | 10 | 30 | 100 |
| 0 | 100 | 85 | 67 | 72 | 100 | 73 | 61 | 56 |
| 50 | 83 | 69 | 61 | 61 | 89 | 65 | 54 | 45 |
| 100 | 80 | 75 | 64 | 61 | 82 | 71 | 59 | 50 |
| 200 | 79 | 70 | 52 | 52 | 58 | 46 | 37 | 22 |

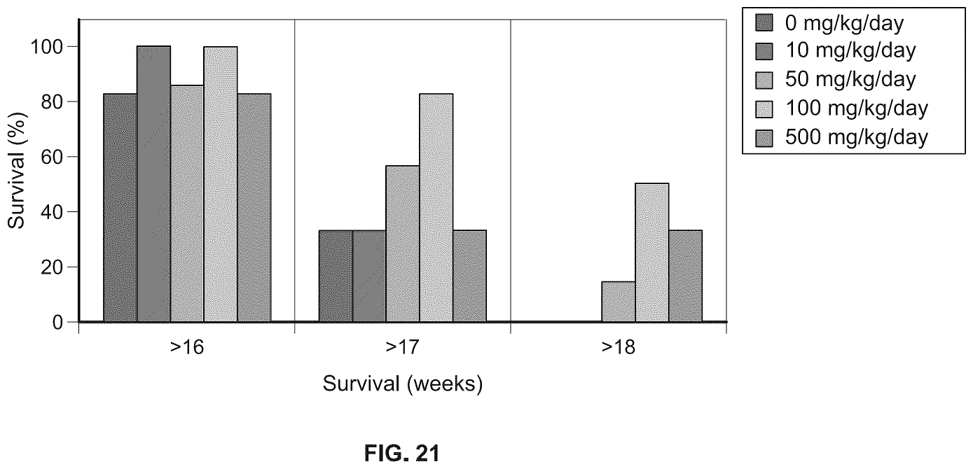

FIG. 22 depicts the change in Filipin staining intensity based on exposure to arimoclomol.

The effects of arimoclomol combined with miglustat on the survival of NPC1^{-/-} mice (at 30 mg/kg/day), miglustat (at 600 mg/kg/day), and the combination of arimoclomol and miglustat (at 30/600 mg/kg/day) were evaluated in Npc1^{-/-} mice and compared to wild type mice and untreated Npc1^{-/-} mice. All comparisons of arimoclomol (p=0.0205), miglustat (p<0.0001), and arimoclomol with miglustat (p<0.0001) showed statistically significant increases in median survival compared to untreated mice. These results are summarized in Table 5:

TABLE 5
Survival Rates in Female Npc1^{-/-} Mutant Mice Treated with Arimoclomol,
Miglustat, or a Combination of Arimoclomol and Miglustat

| | Npc1^{-/-} | Npc1^{-/-} | Npc1^{-/-} | Npc1^{-/-} |
|---|---|---|---|---|
| | untreated | arimoclomol | miglustat | arimoclomol/miglustat |
| Dose (mg/kg/day) | 0 | 30 | 600 | 30/600 |
| Dose (μmol/kg/day) | 0 | 58.8 | 2,740 | 58.8/2740 |
| Median survival (days) | 84.5 | 90 | 120 | 142 |
| % increase in survival compared to | | +6.5% | +42% | +68% |
| untreated | | | | |
| p-value compared to untreated | | 0.0205 | <0.0001 | <0.0001 |
| p-value compared to | <0.0001 | <0.0001 | 0.0007 | |
| arimoclomol/miglustat | | | | |

Miglustat alone demonstrated a larger improvement in median survival than arimoclomol alone (120 vs 90 days for miglustat and arimoclomol, respectively). It should be noted, however, that the miglustat dose was very high (a nearly 2-4 g human equivalent dose in a child or adolescent compared to a typical clinical dose of 100-300 mg miglustat) relative to the arimoclomol dose. Based on the available data, it is therefore difficult to compare the effects of arimoclomol and miglustat and make any inferences regarding survival without knowing the precise dose-response curves for either compound.

When both arimoclomol and miglustat were administered concomitantly, the survival benefit was more than additive as shown in FIG. 22. Arimoclomol alone and miglustat alone increased median survival by 5.5 and 35.5 days, respectively, when compared to untreated animals. Concomitantly administered arimoclomol and miglustat lengthened survival by 57.5 days. This represents more than 2 weeks (16.5 days) of additional survival time compared to the combined individual increases for both compounds.

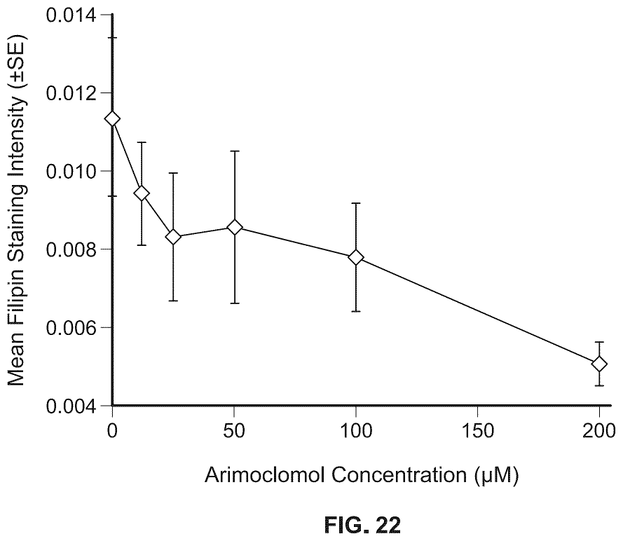

FIG. 23 shows that all mice dosed with either miglustat or with miglustat plus arimoclomol survived longer than all mice of the vehicle control group. Notably, all but 1 animal in the combination group lived longer than all animals in the miglustat group indicating a more than additive effect of arimoclomol and miglustat.

These data are consistent with the results of in vitro studies demonstrated a complimentary effect of arimoclomol and miglustat in the upregulation of the expression of 7 tested CLEAR genes and the HSPA1A gene as well as a complementary increase in cholesterol clearance from lysosomal compartments in human NPC fibroblasts. The finding of longer survival of Npc1−/− mice treated with a combination of arimoclomol and miglustat vs untreated mice established a conceptual bridge between the mechanistic in vitro data and the robust treatment effects of arimoclomol found in the miglustat subgroup of clinical Study 002.

### Example 10—Effects of Arimoclomol and Miglustat Combination Therapy on the Survival of NPC1-Independent and NPC1-Dependent Mice

Across 5 studies, arimoclomol-treated NPC mutant mice (Npc1^{-/-} and Npc1^{nmf164}) consistently outperformed the untreated controls with respect to rearing activity (particularly side rearing events). “Rearing,” as used herein, means a functional behavior in rodents that plays an important role in exploring and interacting with the environment. As such, rearing is a complex behavior that involves aspects such as locomotion, balance, exploratory drive, spatial awareness, cognitive mapping, sequence learning, and decision making. Therefore, the ability to rear is not simply an indicator of muscle function but a broader marker of brain health in rodents. The same brain regions (particularly the cerebellum, hippocampus, and midbrain) that are responsible for controlling rearing behavior in rodents are involved in recruiting muscles during movements related to fine motor function and swallow in humans. Rearing activity in NPC mice is therefore an appropriate indicator of neuronal health in brain regions relevant to functional endpoints like the Fine Motor Skills and Swallow domains of the NPCCSS in human NPC patients.

While the differences between the arimoclomol groups and the untreated groups were typically not statistically significant due to the relatively small numbers of animals and large interindividual variability, the mean differences showed clear overall trends. In 4 of the 5 studies, side or total rearing activity remained comparable to wild type for a longer time in arimoclomol-treated vs untreated mice. Moreover, in all 4 studies in which center rearing was assessed, mutant mice treated with arimoclomol retained some center rearing activity at later time points relative to untreated mice.

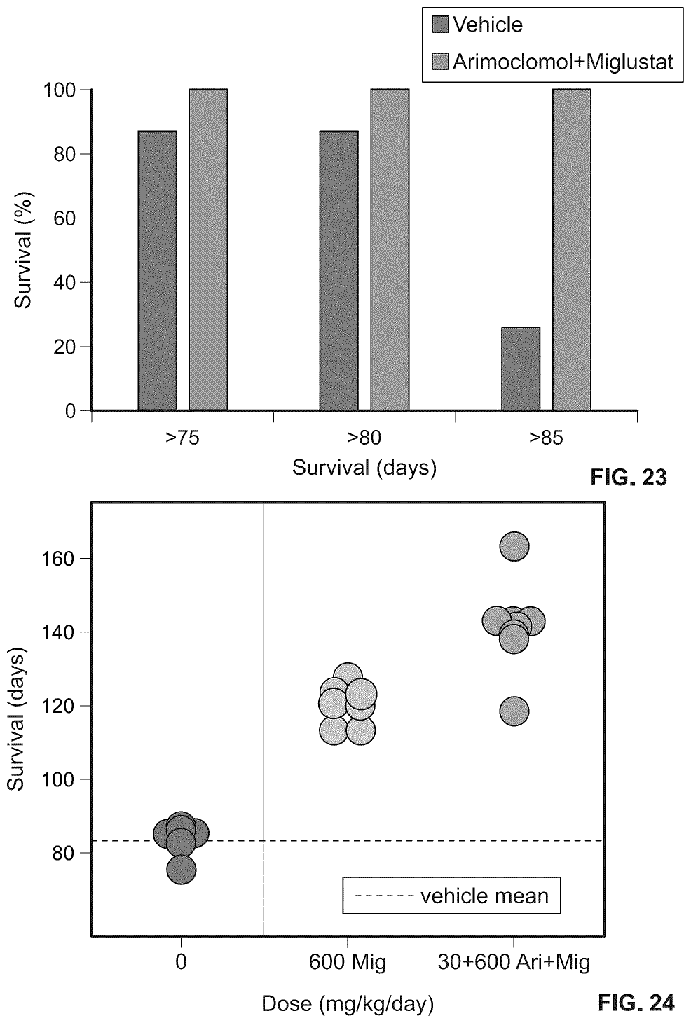

A cross-study summary of the rearing activity at comparable time points and arimoclomol dosages is provided in Table 6 and FIG. 24.

TABLE 6
Rearing Activities in Wild-Type Mice and Untreated, Arimoclomol,
or Arimoclomol/Miglustat-Treated NPC Mice

| | Arimoclomol | | | Arimoclomol | Arimoclomol/miglustat |
|---|---|---|---|---|---|
| Rearing Count, n: | 30 mg/kg/day | | | 100 mg/kg/day | 30/600 mg/kg/day |
| Mouse model | Npc1^{-/-} | Npc1^{-/-} | Npc1^{-/-} | Npc1^{nmf164} | Npc1^{-/-} |
| Study week | 8 | 7 | 9 | 11 | 10 |
| Wild-type | Side: 18.4 | Side: 17.8 | Total: 2.9 | Total: 25.8 | Side: 7.3 |
| | Center: 8.0 | Center: 4.4 | | Center: 10.0 | Center: 2.7 |
| Untreated control | Side: 8.0 | Side: 3.9 | Total: 1.4 | Total: 15.5 | Side: 0.0 |
| | Center: 0.0 | Center: 0.6 | | Center: 0.0 | Center: 0.0 |
| Test article | Side: 17.0 | Side: 7.4 | Total: 2.9 | Total: 23.3 | Side: 8.2 |
| | Center: 10.0 | Center: 2.9 | | Center: 3.2 | Center: 0.8 |

Table represents numerical trends and not statistical analysis.

Rearing activity in NPC mutant mice was typically higher at doses ≥10 mg/kg/day arimoclomol with the largest treatment effects observed between 30 and 100 mg/kg/day when compared to untreated control animals. Differences between arimoclomol-treated and untreated animals were more pronounced near the end of the studies as disease progression resulted in more noticeable effects on rearing behavior in the untreated control group and mice in the arimoclomol groups had been treated for a longer period of time.

While the rearing frequency in arimoclomol-treated mice still decreased over time, the combined data across the in vivo studies showed that this decline was slower in Npc1^{-/-} and Npc1nmf164 mutant mice treated with arimoclomol compared to the vehicle control groups.

A combination treatment of 30 mg/kg/day arimoclomol and 600 mg/kg/day miglustat showed increased rearing events and longer preservation of rearing ability when compared to untreated Npc1^{-/-} mice. The combination also outperformed animals treated only with miglustat and only with arimoclomol. This complimentary effect related to rearing behavior is consistent with the results from multiple in vitro studies that demonstrated complimentary treatment effects of arimoclomol in combination with miglustat with respect to the upregulation of CLEAR genes and reduction in cholesterol accumulation. This finding is also congruent with the increased survival observed in Npc1^{-/-} mice, and ultimately the increased treatment effect found in the miglustat subgroup of NPC patients in clinical.

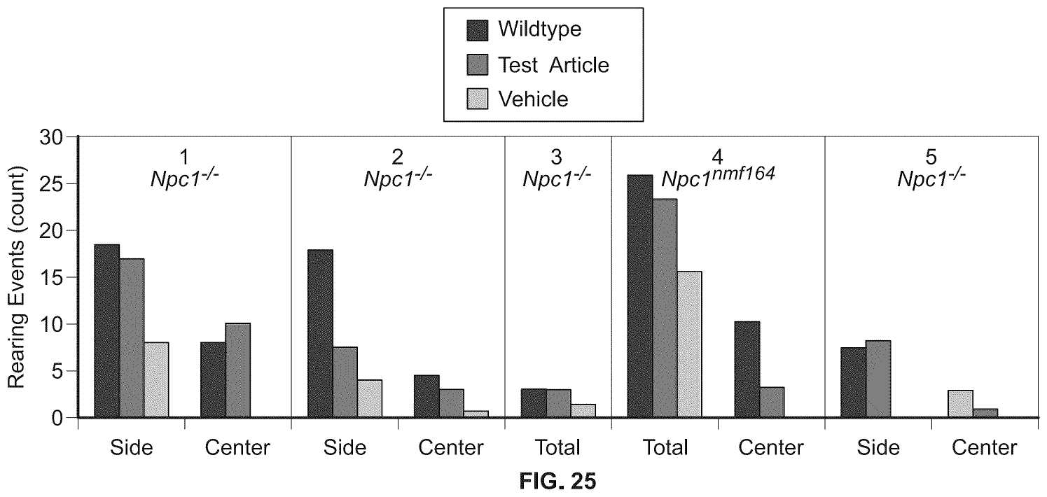

FIG. 25 shows that treatment with arimoclomol increased the number of rearing events in both NPC1-dependent and NPC1-independent mice. NPC1-independent mice treated with arimoclomol had 22% of the rearing events displayed by wild-type mice compared to 3% for NPC1-independent mice that did not receive arimoclomol. Similarly, NPC1-dependent mice treated with arimoclomol displayed 44% of the rearing events displayed by wild-type mice compared to 12%.

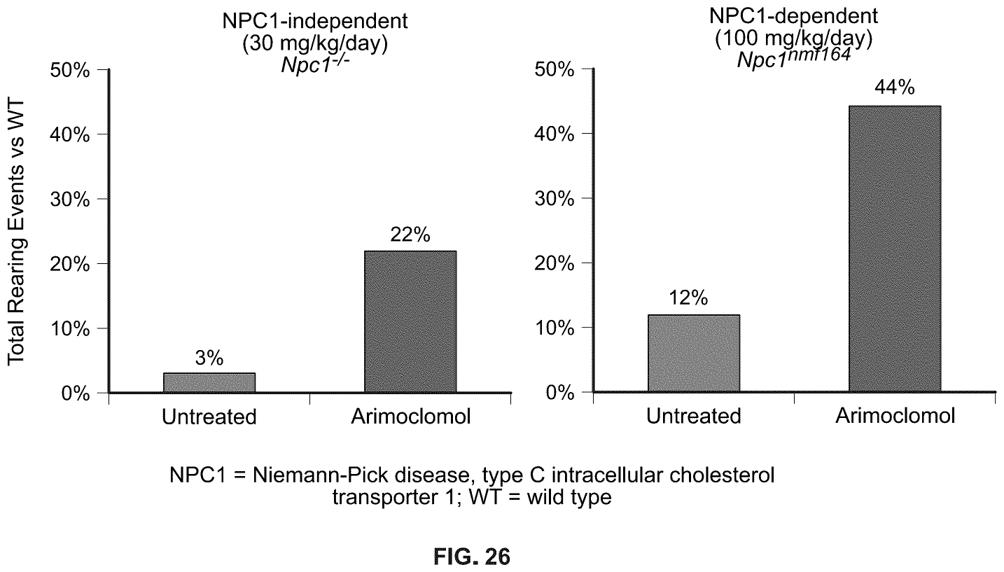

FIG. 26 shows that overall survival rates for mice significantly increased for both NPC1-dependent and NPC1-independent mice. For NPC1-independent mice, nearly twice as many mice survived greater than 85 days when treated with arimoclomol compared to the untreated population. For NPC1-dependent mice, no untreated mice survived beyond the 18 week mark while 50% of mice treated with arimoclomol survived beyond 18 weeks.

Taken together, the data in FIG. 21, through FIG. 26 support that treatment with arimoclomol increased overall neuronal health and longevity in both NPC1-independent and NPC1-dependent mice.

### Example 11—Measurement of Glycosphingolipid and Cholesterol Levels in the Liver and Brain of NPC1^{-/-} and NPC1^{nmf164} Mice

While there are no established biomarkers in humans that are proven to reliably track and correlate with NPC progression or treatment-related improvements, animal models provide the opportunity to investigate tissues and organs that are difficult or impossible to evaluate in humans (e.g., brain). Therefore, biomarkers were assessed in several in vivo studies to 1) potentially confirm certain results of the in vitro studies (e.g., upregulation of NPC1 genes in the brain), and 2) to improve the general understanding of the effects imparted by arimoclomol.

Levels of almost all tested GSLs were numerically lower in the liver and brain of Npc1nmf164 mice treated with 100 mg/kg/day arimoclomol compared to untreated mutant mice (Table 7 and Table 8). Statistically significant differences in the liver were seen for GM2gc, GA2, GM1, and GD1b (Table 9). The decreases of GSLs in the brain were not statistically significant.

TABLE 7
Statistical Analysis of Liver Glycosphingolipid Levels in Wild-type,
Untreated, and Arimoclomol-treated Npc1nmf164 Mice at Age ~12 Weeks

| Arimoclomol | | | | | | | | | | | |
|---|---|---|---|---|---|---|---|---|---|---|---|
| Dose | Glycosphingolipids (Liver) | | | | | | | | | | |
| (mg/kg/day) | GA2 | Gb3 | GD1a | GD1b | GlcCer | GM1 | GM2 | GM3 | GM2gc | GM3gc | LacCer |
| 0 | ã820 | ã93 | ã96 | ã77 | ã110 | ã117 | ã422 | ã688 | ã11051 | ã3761 | ã4155 |
| 100 | ä587 | ä74 | ä78 | ä17 | ä107 | ä76 | ä283 | ä560 | ä7926 | ä2829 | ä3832 |

TABLE 8
Glycosphingolipid Levels Changes in Npc1^{nmf164} Mice at Age ~12
Weeks after Treatment with Arimoclomol vs Untreated

| Arimoclomol | | | | | | | | | | | |
|---|---|---|---|---|---|---|---|---|---|---|---|
| Dose | Glycosphingolipids (Brain) | | | | | | | | | | |
| (mg/kg/day) | GA2 | Gb3 | GD1a | GD1b | GlcCer | GM1 | GM2 | GM3 | GM3gc | GT1b | LacCer |
| 0 | ã416 | ã65 | ã2859 | ã656 | ä41 | ã2421 | ã1964 | ã1290 | ã184 | ã952 | ã7040 |
| 100 | ä346 | ä52 | ä2510 | ä552 | ã70 | ä1903 | ä1454 | ä914 | ä161 | ä874 | ä5919 |

TABLE 9
Statistical Analysis of Liver Glycosphingolipid
Levels in Wild-type, Untreated, and Arimoclomol-treated
Npc1^{nmf164} Mice at Age ~12 Weeks

| | Wild type | Npc1^{nmf164} | Npc1^{nmf164} | p-value |
|---|---|---|---|---|
| | untreated | untreated | arimoclomol | compared |
| Dose (mg/kg/day) | 0 | 0 | 100 | to untreated |
| GM2gc | 2679 | 11051.2 | 7926.2 | 0.025 |
| GA2 | 17.5 | 820.2 | 587.1 | 0.0018 |
| GM1 | 0 | 117.5 | 76.4 | 0.001 |
| GD1b | 0 | 77.1 | 16.7 | 0.0003 |

Unesterified cholesterol levels were assessed in the liver of Npc1^{-/-} mutant mice 7 weeks of age. A small decrease in unesterified cholesterol was observed at 30 mg/kg/day arimoclomol but not at 300 mg/kg/day arimoclomol compared to untreated mice.

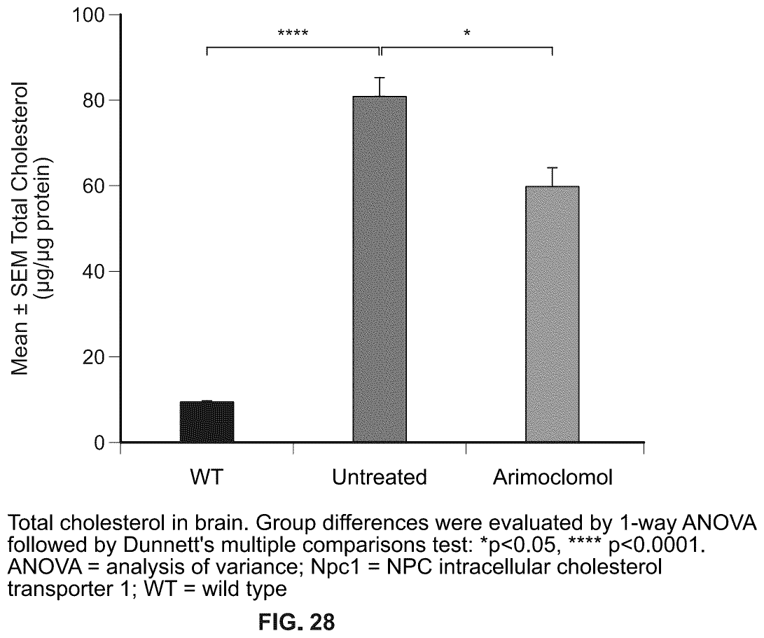

FIG. 28 shows that the total liver cholesterol levels in wild type mice (p<0.0001) and in Npc1^{nmf164} mice treated with 100 mg/kg/day arimoclomol (p=0.0163) were statistically significantly lower compared untreated Npc1^{nmf164} mice. The treatment effect in this study was likely more pronounced relative to study CRO-1202290013 because in Npc1^{nmf164} mice, arimoclomol can engender benefits not only via NPC1-independent pathways, but also by improving maturation and trafficking of the mutant protein. Additionally, the cholesterol levels in Study DOC 2203180048 were evaluated in mice 12 weeks of age vs mice 7 weeks of age in study CRO 1202290013, providing additional time for arimoclomol to accumulate treatment effects.

There was no difference in total brain cholesterol between wild type, untreated, and arimoclomol-treated Npc1^{nmf164} mice. This finding is not unexpected since the brain is mostly an independent and highly regulated closed system with respect to cholesterol metabolism and homeostasis. Indeed, total cholesterol is typically not significantly increased in the NPC1-deficient brain compared to the wild type (Bi and Liao, 2010). The reason for this observation is that the deleterious effects of NPC stem from uneven distribution and not from overall excess of cholesterol in the brain. NPC causes dysfunctional trafficking of cholesterol that becomes sequestered in the cell bodies but is deficient in the axon of neurons (Karten et al., 2003).

### Example 12—NPC1 Protein Concentrations in the Brain and Liver of NPC1^{nmf164} Mice

Analysis of NPC1 protein concentrations in the brain of wild type and Npc^{nmf164} mice showed the presence of 2 different isomers of NPC1. Isoform 1 (>250 kDa) appeared to be a protein dimer (2 protein units) while isoform 2 (˜150 kDa) was assumed to be a monomer (1 protein unit) of NPC1.

The NPC1 dimer is a more complex, mature, and highly glycosylated form of NPC1 that unlike the monomer was resistant to Endoglycosidase (Endo) H degradation (Endo H resistance indicates that newly formed protein has moved out of the endoplasmic reticulum into the Golgi complex) (Brogden et al., 2020). This dimer seemed to be more thermodynamically stable and trafficking competent than the monomer. Consequently, this isoform of NPC1 was able to migrate out of the ER to the late endosomes.

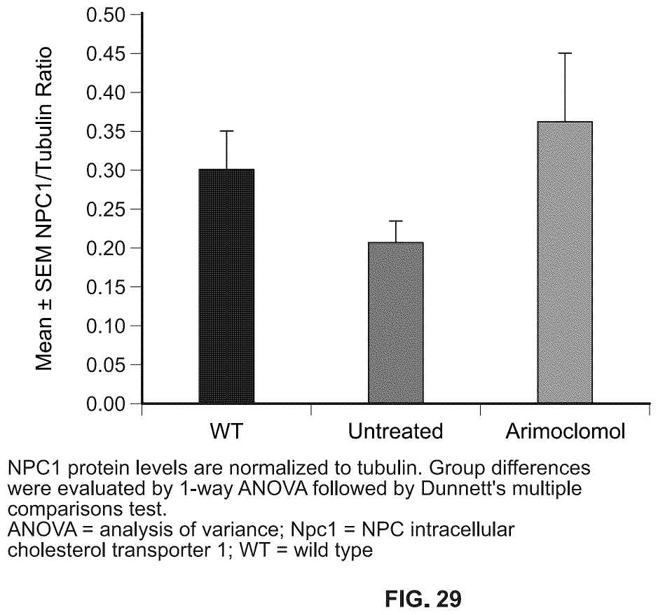

The data indicated a numerically higher mean concentration of the dimeric isoform 1 of NPC1 protein in mutant mice treated with arimoclomol at 100 mg/kg/day vs untreated animals. Moreover, the isoform 1 levels in the arimoclomol group were comparable to concentrations found in the wild type mice. NPC1 protein levels were highly variable in the liver of Npc1^{nmf164} mice treated with arimoclomol at 100 mg/kg/day. While the mean concentrations were numerically higher in the arimoclomol group compared to the untreated group and the wild type group (mean NPC1 to tubulin ratio of 0.36, 0.21 and 0.30, respectively), this difference was not statistically significant (FIG. 29).

### Example 13—Myelin Basic Protein Concentrations in the Brain of NPC1^{nmf164} Mice

As mentioned above, cholesterol plays an important role in the membranes of brain cells. Approximately 80% of the brain cholesterol can be found in myelin that forms a protective sheath around neuronal axons to provide electrical insulation (Bernardo et al., 2021). Another important component of myelin is MBP that interacts with cholesterol to maintain its structural integrity (Deber and Reynolds, 1991). There is evidence that NPC results in myelination defects that leads to neuronal degradation. Decreased expression of MBP has been reported in the Npc1^{nmf164} mouse model which can lead to hypomyelination (Muller et al., 2013; Bernardo et al., 2021).

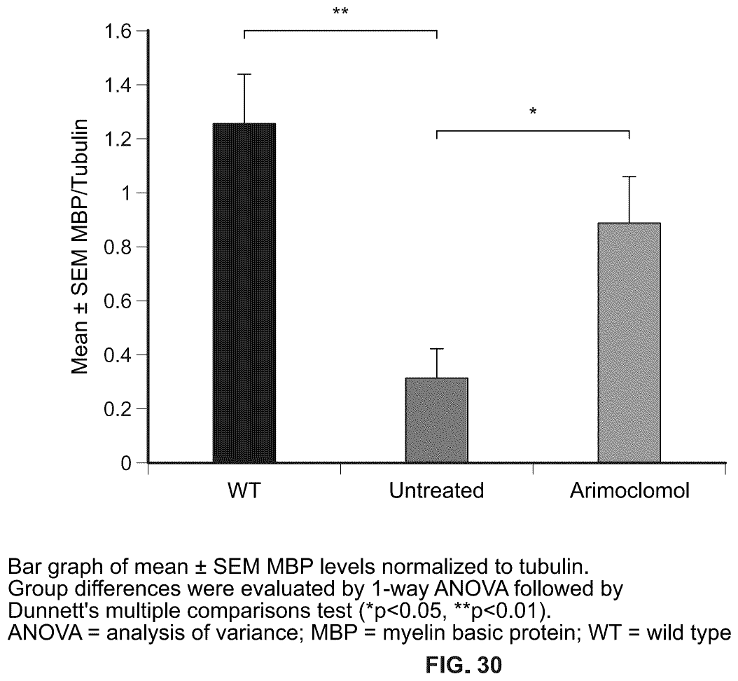

FIG. 30 shows that concentrations of MBP were statistically significantly higher in the brain of Npc^{1nmf164} mutant mice treated with arimoclomol at 100 mg/kg/day compared to untreated mice (FIG. 30). The increased brain levels of MBP in Npc1^{nmf164} mice suggest a slowing effect of arimoclomol on demyelination that would improve neuronal health. Overall, it adds to the body of evidence that arimoclomol can slow disease progression and provide a degree of phenotype stabilization.

Overall, the data generated in the 6 in vivo PD studies show a clear beneficial effect of arimoclomol on survival, the endpoint of most importance. Among the functional endpoints, rearing activity was most consistently improved or maintained in arimoclomol-treated mutant mice when compared to vehicle control animals. Notably, rearing frequency in NPC mutant mice is likely a direct indicator of neuronal health of the cerebellum, hippocampus, and midbrain that are involved in governing fine motor movements including swallowing and chewing in humans. Therefore, the consistent results of improved rearing function across multiple studies and in 2 mouse models provide confirmatory evidence of effectiveness in support of the improvement found in fine motor skills and swallow function in NPC patients treated with arimoclomol vs placebo.

With respect to biomarkers, arimoclomol reduced total cholesterol levels and certain GSLs in the liver of Npc^{nmf164} mice compared to untreated animals. Moreover, the concentration of a mature and trafficking competent isoform of NPC1 protein was numerically elevated in the brain of Npc1^{nmf164} mice treated with arimoclomol. Statistically significantly increased levels of MBP in the brain of Npc1^{nmf164} mice suggest arimoclomol may slow axonal demyelination and neuronal degradation compared to untreated animals.

Consistent with the mechanistic findings of the in vitro studies, the totality of evidence from the in vivo studies showed that arimoclomol produces treatment benefits in animals that are predictive of therapeutic effects in NPC patients. This is consistent with the results of clinical Study 002 that demonstrated symptom stabilization and slowing of disease progression in NPC patients. The arimoclomol effect was amplified in both animals and humans when administered in combination with miglustat.

### Example 14—NPC Disease Progression in Humans

Study 001 was an observational natural history study of 36 patients with NPC, which provided information on NPC disease progression over 6-14 months. Patients completing Study 001 were eligible for enrollment into Study 002. A total of 27 (75.0%) patients from Study 001 continued into Study 002.

Study 002 was the pivotal study of 50 patients with NPC conducted to evaluate efficacy and safety of arimoclomol for the treatment of NPC. The Phase 2/3 study consisted of a 1-year placebo controlled, DB treatment period followed by a 4-year OLE phase where all patients received arimoclomol. In the DB phase of Study 002, patients were randomized 2:1 to arimoclomol or placebo. Thirty-four (34; 68.0%) patients received arimoclomol based on body weight with an estimated equivalence of 372 mg/day for adults, and 16 patients received placebo. A total of 41 patients received arimoclomol in the OLE phase.

Results from the arimoclomol clinical development program for the treatment of NPC demonstrate clinically meaningful slowing of NPC disease progression. And safety data show that arimoclomol is well-tolerated with an acceptable safety profile through 5 years.

Study 002 demonstrated a statistically significant treatment difference in change in 4D-NPCCSS between arimoclomol and placebo in the primary analysis based on the FDA-recommended while-on-treatment estimand. Patients randomized into the arimoclomol group experienced a slower rate of disease progression during the DB phase compared to placebo (FIG. 31 top). In addition, a majority of patients who received arimoclomol did not reach the MCID for disease worsening. Also, the arimoclomol group included more patients with severe disease at baseline, including 3 patients with more rapidly progressing double null mutations.

### Example 15—Placebo-Controlled, Double-Blind Study of the Effectiveness of Arimoclomol on Treating NPC

The study consisted of a 1-year placebo-controlled, DB treatment period followed by a 4-year OLE phase where all patients received arimoclomol. In the DB phase of Study 002, patients were randomized 2:1 to arimoclomol or placebo. Thirty-four (34; 68.0%) patients received arimoclomol based on body weight with an estimated equivalence of 372 mg/day for adults, and 16 patients received placebo. A total of 41 patients received arimoclomol in the OLE phase.

As most patients (39/49 [79.6%]) were also on concomitant miglustat, a separate analysis was carried out to determine the independent treatment effect of arimoclomol in the presence of miglustat. In this analysis all patients in both arms were on concomitant miglustat. The treatment effect of 2.4 points on the 4D-NPCCSS demonstrated that arimoclomol provides a significant benefit for disease progression and symptom scores independent of miglustat

FIG. 31 shows changes in the 4D-NPCCSS scores used to evaluate individual differences in disease trajectory. The top pane of FIG. 1 shows that for patients treated with arimoclomol alone, after 1 year of treatment, 65% of patients stabilized on arimoclomol (10 patients improved, 12 patients had no change) compared with 38% of patients stabilized on placebo (0 patients improved, 6 patients had no change). The bottom pane of FIG. 31 shows that concomitant administration of miglustat resulted in about a 0.5 point decrease (improvement) in the 4D-NPCCSS score over 1 year.

### Example 16—Survival of Npc1^{-/-} Mice after Treatment with Arimoclomol and Miglustat

In this study, the effects of arimoclomol (at 30 mg/kg/day), miglustat (at 600 mg/kg/day), and the combination of arimoclomol and miglustat (at 30/600 mg/kg/day) were evaluated in Npc1^{-/-} mice and compared to wild type mice and untreated Npc1^{-/-} mice. These results are summarized in Table 10.

TABLE 10
Survival Rates in Female Npc1^{nmf164} Mutant Mice Treated
with Arimoclomol (10-500 mg/kg/day) or Vehicle Control

| | | | | Npc1^{-/-} |
|---|---|---|---|---|
| | Npc1^{-/-} | Npc1^{-/-} | Npc1^{-/-} | arimoclomol/ |
| | untreated | arimoclomol | miglustat | miglustat |
| Dose (mg/kg/day): | 0 | 30 | 600 | 30/600 |
| Dose (μmol/kg/day) | 0 | 58.8 | 2,740 | 58.8/2740 |
| Median survival (days) | 84.5 | 90 | 120 | 142 |
| % increase in survival compared to untreated | — | +6.5% | +42% | +68% |
| p-value compared to untreated | — | 0.0205 | <0.0001 | <0.0001 |
| p-value compared to arimoclomol/miglustat | <0.0001 | <0.0001 | 0.0007 | — |

Treatment effect was evaluated by survival curve comparisons using the Log-rank (Mantel-Cox) test.

Miglustat alone demonstrated a larger improvement in median survival than arimoclomol alone (120 vs 90 days for miglustat and arimoclomol, respectively). It should be noted, however, that the miglustat dose was very high (a nearly 2-4 g human equivalent dose in a child or adolescent compared to a typical clinical dose of 100-300 mg miglustat) relative to the arimoclomol dose. Based on the available data, it is therefore difficult to compare the effects of arimoclomol and miglustat and make any inferences regarding survival without knowing the precise dose-response curves for either compound.

When both arimoclomol and miglustat were administered concomitantly, the survival benefit was more than additive (Table 10). Arimoclomol alone and miglustat alone increased median survival by 5.5 and 35.5 days, respectively, when compared to untreated animals. Concomitantly administered arimoclomol and miglustat lengthened survival by 57.5 days. This represents more than 2 weeks (16.5 days) of additional survival time compared to the combined individual increases for both compounds.

All comparisons of arimoclomol (p=0.0205), miglustat (p<0.0001), and arimoclomol with miglustat (p<0.0001) showed statistically significant increases in median survival compared to untreated mice.

### EMBODIMENTS

1. A method of increasing removal of lysosomal cholesterol from cells of a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

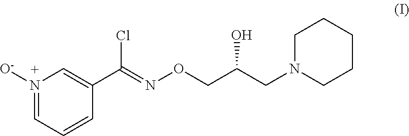

or a pharmaceutically acceptable salt thereof, where increasing the activation of the at least one transcription factor increases the removal of lysosomal cholesterol from the cells of the patient relative to a patient not administered a therapeutically effective amount of the compound of Formula I, or a pharmaceutically acceptable salt thereof.
2. The method of 1, wherein the lysosomal cholesterol is unesterified cholesterol.
3. The method of 1 or 2, wherein the patient has a disease associated with impaired cholesterol trafficking.
4. The method of 3 wherein the disease is NPC (Niemann-Pick, Type C disease).
5. The method of 4, wherein the NPC disease is selected from the group consisting of NPC, Type 1 (NPC-1) and NPC Type 2 (NPC2).
6. The method of 5, wherein the NPC disease is NPC1.
7. The method of 1-6, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
8. The method of 7, wherein the pharmaceutically acceptable salt is citrate.
9. The method of 8, wherein the compound of Formula I is arimoclomol citrate.
10. The method of 9, wherein the compound having structure of formula (I) is N-[(2R,Z)-2-hydroxy-3-(1-piperidyl)propoxy]pyridine-3-carboximidoyl chloride, 1-oxide citrate.
11. The method of 1-10, wherein the therapeutically effective amount of the compound having the structure of Formula I is a daily dosage of about 1 to about 30 mg/kg/day.
12. The method of 11, wherein the daily dosage ranges from about 0.1 mg to about 400 mg.
13. The method of 12, wherein the daily dosage is administered in about 1-3 unit doses per day.
14. The method of 13, wherein the daily dosage is administered in 3 unit doses per day.
15. The method of 13 or 14, wherein the unit doses are selected from the group consisting of 31 mg, 47 mg, 62 mg, 93 mg, and 124 mg.
16. The method of 1, wherein the composition is combined with soft food prior to administration.
17. The method of 1, wherein the composition is dispersed in water prior to administration.
18. The method of 1, wherein the composition is an oral dosage formulation.
19. The method of 18, wherein the oral dosage formulation is a solid oral dosage formulation.
20. The method of 19, wherein the solid oral dosage is selected from the group consisting of tablets, capsules, caplets, films, and powders.
21. The method of 19, wherein the solid oral dosage form further comprises one or more excipients.
22. The method of 18, wherein the oral dosage formulation is a liquid oral dosage formulation.
23. The method of 22, wherein the liquid oral dosage formulation further comprises a thickening agent.
24. The method of 22, wherein the liquid oral dosage formulation is formulated for administration via a feeding tube or gastric tube.
25. The method of 1, wherein the composition is formulated for parenteral administration.
26. A method of increasing removal of lysosomal cholesterol from cells of a patient/subject with moderate to severe renal impairment, the method comprising: determining the Estimated Glomerular Filtration Rate (eGFR) of the subject/patient; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

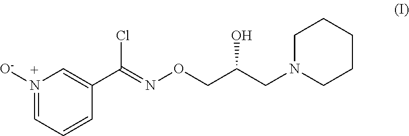

or a pharmaceutically acceptable salt thereof, where increasing the activation of the at least one transcription factor increases the removal of lysosomal cholesterol from the cells of the patient relative to a patient not being administered a therapeutically effective amount of the compound having a structure of Formula I, or a pharmaceutically acceptable salt thereof.
27. The method of 26, wherein the Estimated Glomerular Filtration Rate (eGFR) of the subject/patient with moderate to severe renal impairment ranges from 15 mL/min to 50 mL/min.
28. The method of 26, wherein the lysosomal cholesterol is unesterified cholesterol.
29. The method of 26-28, wherein the patient has a disease associated with impaired cholesterol trafficking.
30. The method of 29, wherein the disease is NPC (Niemann-Pick, Type C disease).
31. The method of 30, wherein the NPC disease is selected from the group consisting of NPC, Type 1 (NPC-1) and NPC Type 2 (NPC2).
32. The method of 31, wherein the NPC disease is NPC1.
33. The method of 26-32, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
34. The method of 33, wherein the pharmaceutically acceptable salt is citrate.
35. The method of 34, wherein the compound of Formula I is arimoclomol citrate.
36. The method of 35, wherein the compound having structure of formula (I) is N-[(2R,Z)-2-hydroxy-3-(1-piperidyl)propoxy]pyridine-3-carboximidoyl chloride, 1-oxide citrate.
37. The method of 26-36, wherein the therapeutically effective amount of the compound having the structure of Formula I is a daily dosage of about 1 to about 15 mg/kg/day.
38. The method of 37, wherein the daily dosage ranges from about 0.1 mg to about 250 mg per day.
39. The method of 38, wherein the daily dosage is administered in about 1-3 unit doses per day.
40. The method of 39, wherein the daily dosage is administered in 2 unit doses per day.
41. The method of 40, wherein the unit doses are selected from the group consisting of 31 mg, 47 mg, 62 mg, 93 mg, and 124 mg
42. The method of 26, wherein the composition is combined with soft food prior to administration.
43. The method of 26, wherein the composition is dispersed in water prior to administration.
44. The method of 26, wherein the composition is an oral dosage formulation.
45. The method of 44, wherein the oral dosage formulation is a solid oral dosage formulation.
46. The method of 45, wherein the solid oral dosage is selected from the group consisting of tablets, capsules, caplets, films, and powders.
47. The method of 45, wherein the solid oral dosage form further comprises one or more excipients.
48. The method of 44, wherein the oral dosage formulation is a liquid oral dosage formulation.
49. The method of 48, wherein the liquid oral dosage formulation further comprises a thickening agent.
50. The method of 48, wherein the liquid oral dosage formulation is formulated for administration via a feeding tube or gastric tube.
51. The method of 26, wherein the composition is formulated for parenteral administration.
52. A method of increasing removal of endosomal cholesterol from cells of a patient in need thereof, the method comprising: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

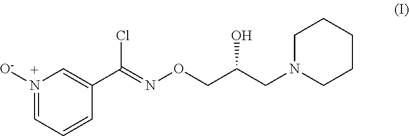

or a pharmaceutically acceptable salt thereof, where increasing the activation of the at least one transcription factor increases the removal of endosomal cholesterol from the cells of the patient relative to a patient not being administered a therapeutically effective amount of the compound having a structure of Formula I, or a pharmaceutically acceptable salt thereof.
53. The method of 52, wherein the endosomal cholesterol is unesterified cholesterol.
54. The method of 52 or 53, wherein the patient has a disease associated with impaired cholesterol trafficking.
55. The method of 54 wherein the disease is NPC (Niemann-Pick, Type C disease).
56. The method of 55, wherein the NPC disease is selected from the group consisting of NPC, Type 1 (NPC-1) and NPC Type 2 (NPC2).
57. The method of 56, wherein the NPC disease is NPC1.
58. The method of 52-57, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
59. The method of 58, wherein the pharmaceutically acceptable salt is citrate.
60. The method of 59, wherein the compound of Formula I is arimoclomol citrate.
61. The method of 60, wherein the compound having structure of formula (I) is N-[(2R,Z)-2-hydroxy-3-(1-piperidyl)propoxy]pyridine-3-carboximidoyl chloride, 1-oxide citrate.
62. The method of 52-61, wherein the therapeutically effective amount of the compound having the structure of Formula I is a daily dosage of about 1 mg/kg/day to about 30 mg/kg/day.
63. The method of 62, wherein the daily dosage ranges from about 0.1 mg to about 400 mg.
64. The method of 63, wherein the daily dosage is administered in about 1-3 unit doses per day.
65. The method of 64, wherein the daily dosage is administered in 3 unit doses per day.
66. The method of 65, wherein the unit dosage is selected from the group consisting of 31 mg, 47 mg, 62 mg, 93 mg, and 124 mg.
67. The method of 52, wherein the composition is combined with soft food prior to administration.
68. The method of 52, wherein the composition is dispersed in water prior to administration.
69. The method of 52, wherein the composition is an oral dosage formulation.
70. The method of 69, wherein the oral dosage formulation is a solid oral dosage formulation.
71. The method of 70, wherein the solid oral dosage is selected from the group consisting of tablets, capsules, caplets, films, and powders.
72. The method of 70, wherein the solid oral dosage form further comprises one or more excipients.
73. The method of 69, wherein the oral dosage formulation is a liquid oral dosage formulation.
74. The method of 73, wherein the liquid oral dosage formulation further comprises a thickening agent.
75. The method of 73, wherein the liquid oral dosage formulation is formulated for administration via a feeding tube or gastric tube.
76. The method of 52, wherein the composition is formulated for parenteral administration.
77. A method of removing endosomal cholesterol from cells of a patient/subject with moderate to severe renal impairment, the method comprising the steps of: determining the Estimated Glomerular Filtration Rate (eGFR) of the subject/patient; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

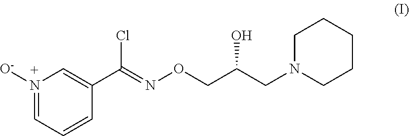

or a pharmaceutically acceptable salt thereof, where increasing the activation of the at least one transcription factor increases the removal of endosomal cholesterol from the cells of the patient relative to a patient not being administered a therapeutically effective amount of the compound having a structure of Formula I, or a pharmaceutically acceptable salt thereof.
78. The method of 77, wherein the Estimated Glomerular Filtration Rate (eGFR) of the subject/patient with moderate to severe renal impairment ranges from 15 mL/min to 50 mL/min.
79. The method of 77, wherein the endosomal cholesterol is unesterified cholesterol.
80. The method of 77-79, wherein the patient has a disease associated with impaired cholesterol trafficking.
81. The method of 80, wherein the disease is NPC (Niemann-Pick, Type C disease).
82. The method of 81, wherein the NPC disease is selected from the group consisting of NPC, Type 1 (NPC-1) and NPC Type 2 (NPC2).
83. The method of 82, wherein the NPC disease is NPC1.
84. The method of 77-83, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
85. The method of 84, wherein the pharmaceutically acceptable salt is citrate.
86. The method of 85, wherein the compound of Formula I is arimoclomol citrate.
87. The method of 86, wherein the compound having structure of formula (I) is N-[(2R,Z)-2-hydroxy-3-(1-piperidyl)propoxy]pyridine-3-carboximidoyl chloride, 1-oxide citrate.
88. The method of 77-87, wherein the therapeutically effective amount of the compound having the structure of Formula I is a daily dosage of about 1 mg/kg/day to about 15 mg/kg/day.
89. The method of 88, wherein the daily dosage ranges from about 0.1 mg to about 250 mg per day.
90. The method of 89, wherein the daily dosage is administered in about 1-3 unit doses per day.
91. The method of 90, wherein the daily dosage is administered in 2 unit doses per day.
92. The method of 91, wherein the unit dosage is selected from the group consisting of 31 mg, 47 mg, 62 mg, 93 mg, and 124 mg.
93. The method of 77, wherein the composition is combined with soft food prior to administration.
94. The method of 77, wherein the composition is dispersed in water prior to administration.
95. The method of 77, wherein the composition is an oral dosage formulation.
96. The method of 95, wherein the oral dosage formulation is a solid oral dosage formulation.
97. The method of 96, wherein the solid oral dosage is selected from the group consisting of tablets, capsules, caplets, films, and powders.
98. The method of 96, wherein the solid oral dosage form further comprises one or more excipients.
99. The method of 95, wherein the oral dosage formulation is a liquid oral dosage formulation.
100. The method of 99, wherein the liquid oral dosage formulation further comprises a thickening agent.
101. The method of 99, wherein the liquid oral dosage formulation is formulated for administration via a feeding tube or gastric tube.
102. The method of 77, wherein the composition is formulated for parenteral administration.
103. A method of increasing cellular production of NPC1 protein in a patient with a mutant Niemann-Pick type-C Protein 1 (NPC1) with decreased functionality compared to wild-type NPC1 protein, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

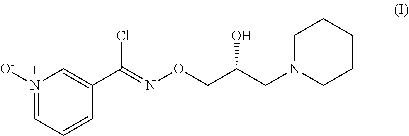

or a pharmaceutically acceptable salt thereof, where increasing the activation of the at least one transcription factor increases cellular production of the mutant NPC1 protein having reduced functionality compared to wild-type NPC1 protein relative to a patient not being administered a therapeutically effective amount of the compound having a structure of Formula I, or a pharmaceutically acceptable salt thereof.
104. The method of 103, wherein the patient with a mutant Niemann-Pick type-C Protein 1 with decreased functionality compared to wild-type NPC1 protein has Niemann-Pick disease.
105. The method of 104, wherein the Niemann-Pick disease is Nieman-Pick disease, type-C and the Nieman-Pick disease, type-C, is selected from the group consisting of NPC-1 and NPC-2.
106. The method of 105, wherein the NPC disease is NPC1.
107. The method of 103-106, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
108. The method of 107, wherein the pharmaceutically acceptable salt is citrate.
109. The method of 108, wherein the compound of Formula I is arimoclomol citrate.
110. The method of 109, wherein the compound having structure of Formula (I) is N-[(2R,Z)-2-hydroxy-3-(1-piperidyl)propoxy]pyridine-3-carboximidoyl chloride, 1-oxide citrate.
111. The method of 103-110, wherein the therapeutically effective amount of the compound having the structure of Formula I is a daily dosage of about 1 mg/kg/day to about 30 mg/kg/day.
112. The method of 111, wherein the daily dosage ranges from about 0.1 mg to about 400 mg.
113. The method of 112, wherein the daily dosage is administered in about 1-3 unit doses per day.
114. The method of 113, wherein the daily dosage is administered in 3 unit doses per day.
115. The method of 114, wherein the unit dosage is selected from the group consisting of 31 mg, 47 mg, 62 mg, 93 mg, and 124 mg.
116. The method of 103, wherein the composition is combined with soft food prior to administration.
117. The method of 103, wherein the composition is dispersed in water prior to administration.
118. The method of 103, wherein the composition is an oral dosage formulation.
119. The method of 118, wherein the oral dosage formulation is a solid oral dosage formulation.
120. The method of 119, wherein the solid oral dosage is selected from the group consisting of tablets, capsules, caplets, films, and powders.
121. The method of 119, wherein the solid oral dosage form further comprises one or more excipients.
122. The method of 118, wherein the oral dosage formulation is a liquid oral dosage formulation.
123. The method of 122, wherein the liquid oral dosage formulation further comprises a thickening agent.
124. The method of 122, wherein the liquid oral dosage formulation is formulated for administration via a feeding tube or gastric tube.
125. The method of 103, wherein the composition is formulated for parenteral administration.
126. A method of increasing cellular production of NPC1 protein in a patient with a mutant Niemann-Pick type-C Protein 1 (NPC1) with decreased functionality, and moderate to severe renal impairment, the method comprising the steps of: determining the Estimated Glomerular Filtration Rate (eGFR) of the patient with a mutant Niemann-Pick type-C Protein 1 (NPC1) with decreased functionality; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

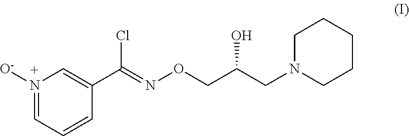

or a pharmaceutically acceptable salt thereof; where increasing the activation of the at least one transcription factor increases cellular production of the mutant NPC1 protein having reduced functionality relative to a patient not being administered a therapeutically effective amount of the compound having a structure of Formula I, or a pharmaceutically acceptable salt thereof.
127. The method of 126, wherein the Estimated Glomerular Filtration Rate (eGFR) of the subject/patient with moderate to severe renal impairment ranges from 15 mL/min to 50 mL/min.
128. The method of 127, wherein the disease or disorder resulting in mutant Niemann-Pick type-C Protein 1 (NPC1) with deceased functionality is Niemann-Pick disease.
129. The method of 128, wherein the Niemann-Pick disease is selected from the group consisting of NPC-1 and NPC-2
130. The method of 129, wherein the NPC disease is NPC1.
131. The method of 126-130, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
132. The method of 131, wherein the pharmaceutically acceptable salt is citrate.
133. The method of 132, wherein the compound of Formula I is arimoclomol citrate.
134. The method of 133, wherein the compound having structure of formula (I) is N-[(2R,Z)-2-hydroxy-3-(1-piperidyl)propoxy]pyridine-3-carboximidoyl chloride, 1-oxide citrate.
135. The method of 126-134, wherein the therapeutically effective amount of the compound having the structure of Formula I is a daily dosage of about 1 mg/kg/day to about 15 mg/kg/day.
136. The method of 135, wherein the daily dosage ranges from about 0.1 mg to about 250 mg per day.
137. The method of 136, wherein the daily dosage is administered in about 1-3 unit doses per day.
138. The method of 137, wherein the daily dosage is administered in 2 unit doses per day.
139. The method of 138, wherein the unit dosage is selected from the group consisting of 31 mg, 47 mg, 62 mg, 93 mg, and 124 mg.
140. The method of 126, wherein the composition is combined with soft food prior to administration.
141. The method of 126, wherein the composition is dispersed in water prior to administration.
142. The method of 126, wherein the composition is an oral dosage formulation.
143. The method of 142, wherein the oral dosage formulation is a solid oral dosage formulation.
144. The method of 143, wherein the solid oral dosage is selected from the group consisting of tablets, capsules, caplets, films, and powders.
145. The method of 143, wherein the solid oral dosage form further comprises one or more excipients.
146. The method of 142, wherein the oral dosage formulation is a liquid oral dosage formulation.
147. The method of 146, wherein the liquid oral dosage formulation further comprises a thickening agent.
148. The method of 146, wherein the liquid oral dosage formulation is formulated for administration via a feeding tube or gastric tube.
149. The method of 126, wherein the composition is formulated for parenteral administration.
150. A method for improving at least one 4D-NPCCSS scale domain score in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

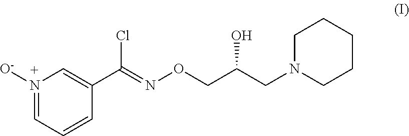

or a pharmaceutically acceptable salt thereof, where increasing the activation of the at least one transcription factor decreases at least one 4D-NPCCSS domain score, wherein the at least one 4D-NPCCSS domain is selected from ambulation, fine motor skills, and speech relative to a patient not being administered a therapeutically effective amount of the compound having a structure of Formula I, or a pharmaceutically acceptable salt thereof.
151. The method of 150, wherein the patient has a disease associated with impaired cholesterol trafficking.
152. The method of 151 wherein the disease is NPC (Niemann-Pick, Type C disease).
153. The method of 152, wherein the NPC disease is selected from the group consisting of NPC, Type 1 (NPC-1) and NPC Type 2 (NPC2).
154. The method of 153, wherein the NPC disease is NPC1.
155. The method of 150-154, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
156. The method of 155, wherein the pharmaceutically acceptable salt is citrate.
157. The method of 156, wherein the compound of Formula I is arimoclomol citrate.
158. The method of 159, wherein the compound having structure of formula (I) is N-[(2R,Z)-2-hydroxy-3-(1-piperidyl)propoxy]pyridine-3-carboximidoyl chloride, 1-oxide citrate.
159. The method of 150-158, wherein the therapeutically effective amount of the compound having the structure of Formula I is a daily dosage of about 1 mg/kg/day to about 30 mg/kg/day.
160. The method of 159, wherein the daily dosage ranges from about 0.1 mg to about 400 mg.
161. The method of 160, wherein the daily dosage is administered in about 1-3 unit doses per day.
162. The method of 161, wherein the daily dosage is administered in 3 unit doses per day.
163. The method of 162, wherein the unit dosage is selected from the group consisting of 31 mg, 47 mg, 62 mg, 93 mg, and 124 mg.
164. The method of 150, wherein the composition is combined with soft food prior to administration.
165. The method of 150, wherein the composition is dispersed in water prior to administration.
166. The method of 150, wherein the composition is an oral dosage formulation.
167. The method of 166, wherein the oral dosage formulation is a solid oral dosage formulation.
168. The method of 167, wherein the solid oral dosage is selected from the group consisting of tablets, capsules, caplets, films, and powders.
169. The method of 167, wherein the solid oral dosage form further comprises one or more excipients.
170. The method of 166, wherein the oral dosage formulation is a liquid oral dosage formulation.
171. The method of 170, wherein the liquid oral dosage formulation further comprises a thickening agent.
172. The method of 170, wherein the liquid oral dosage formulation is formulated for administration via a feeding tube or gastric tube.
173. The method of 150, wherein the composition is formulated for parenteral administration.
174. The method according to 150-173, wherein the administration of the compound having a structure of Formula I results in a decrease in at least one scale score selected from SARA GAIT, SARA Finger chase, SARA Nose-finger test, SARA Fast alternating hand movements, 9-HPT, and SARA Speech disturbance.
175. A method for improving at least one 4D-NPCCSS scale domain score in a patient in need thereof with moderate to severe renal impairment, the method comprising the steps of: determining the Estimated Glomerular Filtration Rate (eGFR) of the patient with a mutant Niemann-Pick type-C Protein 1 (NPC1) with decreased functionality; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

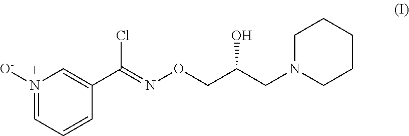

or a pharmaceutically acceptable salt thereof, where increasing the activation of the at least one transcription factor decreases at least one 4D-NPCCSS domain score, wherein the at least one 4D-NPCCSS domain is selected from ambulation, fine motor skills, and speech relative to a patient not being administered a therapeutically effective amount of the compound having a structure of Formula I, or a pharmaceutically acceptable salt thereof.
176. The method of 175, wherein the Estimated Glomerular Filtration Rate (eGFR) of the subject/patient with moderate to severe renal impairment ranges from 15 mL/min to 50 mL/min.
177. The method of 176, wherein the patient has a disease associated with impaired cholesterol trafficking.
178. The method of 177, wherein the disease is NPC (Niemann-Pick, Type C disease).
179. The method of 178, wherein the NPC disease is selected from the group consisting of NPC, Type 1 (NPC-1) and NPC Type 2 (NPC2).
180. The method of 179, wherein the NPC disease is NPC1.
181. The method of 175-180, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
182. The method of 181, wherein the pharmaceutically acceptable salt is citrate.
183. The method of 182, wherein the compound of Formula I is arimoclomol citrate.
184. The method of 183, wherein the compound having structure of formula (I) is N-[(2R,Z)-2-hydroxy-3-(1-piperidyl)propoxy]pyridine-3-carboximidoyl chloride, 1-oxide citrate.
185. The method of 175-184, wherein the therapeutically effective amount of the compound having the structure of Formula I is a daily dosage of about 1 mg/kg/day to about 30 mg/kg/day.
186. The method of 185, wherein the daily dosage ranges from about 0.1 mg to about 250 mg.
187. The method of 186, wherein the daily dosage is administered in about 1-3 unit doses per day.
188. The method of 187, wherein the daily dosage is administered in 2 unit doses per day.
189. The method of 188, wherein the unit dosage is selected from the group consisting of 31 mg, 47 mg, 62 mg, 93 mg, and 124 mg.
190. The method of 175, wherein the composition is combined with soft food prior to administration.
191. The method of 175, wherein the composition is dispersed in water prior to administration.
192. The method of 175, wherein the composition is an oral dosage formulation.
193. The method of 192, wherein the oral dosage formulation is a solid oral dosage formulation.
194. The method of 193, wherein the solid oral dosage is selected from the group consisting of tablets, capsules, caplets, films, and powders.
195. The method of 193, wherein the solid oral dosage form further comprises one or more excipients.
196. The method of 192, wherein the oral dosage formulation is a liquid oral dosage formulation.
197. The method of 196, wherein the liquid oral dosage formulation further comprises a thickening agent.
198. The method of 196, wherein the liquid oral dosage formulation is formulated for administration via a feeding tube or gastric tube.
199. The method of 175, wherein the composition is formulated for parenteral administration.
200. The method according to 175-199, wherein the administration of the compound having a structure of Formula I results in a decrease in at least one scale score selected from SARA GAIT, SARA Finger chase, SARA Nose-finger test, SARA Fast alternating hand movements, 9-HPT, and SARA Speech disturbance.
201. A method for reducing NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

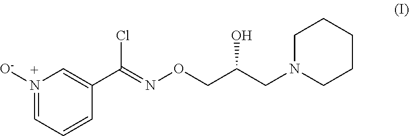

or a pharmaceutically acceptable salt thereof, wherein administering the compound having a structure of Formula I reduces the progression of NPC disease as compared to a placebo group.
202. The method of 201, wherein the NPC disease is selected from the group consisting of NPC, Type 1 (NPC-1) and NPC Type 2 (NPC2).
203. The method of 202, wherein the NPC disease is NPC1.
204. The method of 201-203, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
205. The method of 204, wherein the pharmaceutically acceptable salt is citrate.
206. The method of 205, wherein the compound of Formula I is arimoclomol citrate.
207. The method of 206, wherein the compound having structure of formula (I) is N-[(2R,Z)-2-hydroxy-3-(1-piperidyl)propoxy]pyridine-3-carboximidoyl chloride, 1-oxide citrate.
208. The method of 201-207, wherein the therapeutically effective amount of the compound having the structure of Formula I is a daily dosage of about 1 mg/kg/day to about 30 mg/kg/day.
209. The method of 208, wherein the daily dosage ranges from about 0.1 mg to about 400 mg.
210. The method of 209, wherein the daily dosage is administered in about 1-3 unit doses per day.
211. The method of 210, wherein the daily dosage is administered in 3 unit doses per day.
212. The method of 211, wherein the unit dosage is selected from the group consisting of 31 mg, 47 mg, 62 mg, 93 mg, and 124 mg.
213. The method of 201, wherein the composition is combined with soft food prior to administration.
214. The method of 201, wherein the composition is dispersed in water prior to administration.
215. The method of claim 201, wherein the composition is an oral dosage formulation.
216. The method of 215, wherein the oral dosage formulation is a solid oral dosage formulation.
217. The method of 216, wherein the solid oral dosage is selected from the group consisting of tablets, capsules, caplets, films, and powders.
218. The method of 216, wherein the solid oral dosage form further comprises one or more excipients.
219. The method of 215, wherein the oral dosage formulation is a liquid oral dosage formulation
220. The method of 219, wherein the liquid oral dosage formulation further comprises a thickening agent.
221. The method of 219, wherein the liquid oral dosage formulation is formulated for administration via a feeding tube or gastric tube.
222. The method of 201, wherein the composition is formulated for parenteral administration.
223. The method according to 201-222, wherein the administration of the compound having a structure of Formula I results in a decrease in at least one scale score selected from SARA GAIT, SARA Finger chase, SARA Nose-finger test, SARA Fast alternating hand movements, 9-HPT, and SARA Speech disturbance.
224. The method according to 201-223, wherein the reduction in NPC disease progression is measured by a decrease in at least one scale score selected from SARA GAIT, SARA Finger chase, SARA Nose-finger test, SARA Fast alternating hand movements, 9-HPT, and SARA Speech disturbance.
225. A method for reducing NPC disease progression in a patient in need thereof with moderate to severe renal impairment, the method comprising the steps of: determining the Estimated Glomerular Filtration Rate (eGFR) of the patient with a mutant Niemann-Pick type-C Protein 1 (NPC1) with decreased functionality; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

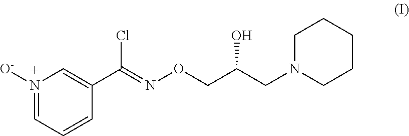

or a pharmaceutically acceptable salt thereof, wherein administering the compound having a structure of Formula I reduces the progression of NPC disease as compared to a placebo group.
226. The method of 225, wherein the Estimated Glomerular Filtration Rate (eGFR) of the subject/patient with moderate to severe renal impairment ranges from 15 mL/min to 50 mL/min.
227. The method of 226, wherein the NPC disease is NPC1.
228. The method of 225-227, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
229. The method of 228, wherein the pharmaceutically acceptable salt is citrate.
230. The method of 229, wherein the compound of Formula I is arimoclomol citrate.
231. The method of 230, wherein the compound having structure of formula (I) is N-[(2R,Z)-2-hydroxy-3-(1-piperidyl)propoxy]pyridine-3-carboximidoyl chloride, 1-oxide citrate.
232. The method of 225-231, wherein the therapeutically effective amount of the compound having the structure of Formula I is a daily dosage of about 1 mg/kg/day to about 30 mg/kg/day.
233. The method of 232, wherein the daily dosage ranges from about 0.1 mg to about 250 mg.
234. The method of 233, wherein the daily dosage is administered in about 1-3 unit doses per day.
235. The method of 234, wherein the daily dosage is administered in 2 unit doses per day.
236. The method of 235, wherein the unit dosage is selected from the group consisting of 31 mg, 47 mg, 62 mg, 93 mg, and 124 mg.
237. The method of 225, wherein the composition is combined with soft food prior to administration.
238. The method of 225, wherein the composition is dispersed in water prior to administration.
239. The method of 225, wherein the composition is an oral dosage formulation.
240. The method of 239, wherein the oral dosage formulation is a solid oral dosage formulation.
241. The method of 240, wherein the solid oral dosage is selected from the group consisting of tablets, capsules, caplets, films, and powders.
242. The method of 240, wherein the solid oral dosage form further comprises one or more excipients.
243. The method of 239, wherein the oral dosage formulation is a liquid oral dosage formulation.
244. The method of 243, wherein the liquid oral dosage formulation further comprises a thickening agent.
245. The method of 243, wherein the liquid oral dosage formulation is formulated for administration via a feeding tube or gastric tube.
246. The method of 225, wherein the composition is formulated for parenteral administration
247. The method according to 225-246, wherein the administration of the compound having a structure of Formula I results in a decrease in at least one scale score selected from SARA GAIT, SARA Finger chase, SARA Nose-finger test, SARA Fast alternating hand movements, 9-HPT, and SARA Speech disturbance.
248. The method according to 225-247, wherein the reduction in NPC disease progression is measured by a decrease in at least one scale score selected from SARA GAIT, SARA Finger chase, SARA Nose-finger test, SARA Fast alternating hand movements, 9-HPT, and SARA Speech disturbance.
249. A method of delaying NPC disease progression in a patient in need thereof, the method comprising the steps of: increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

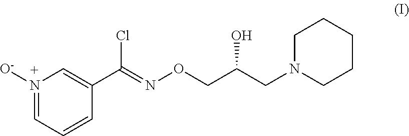

or a pharmaceutically acceptable salt thereof, wherein administering the compound having a structure of Formula I delays the progression of NPC disease as compared to a placebo group.
250. The method of 249, wherein the NPC disease is selected from the group consisting of NPC, Type 1 (NPC-1) and NPC Type 2 (NPC2).
251. The method of 250, wherein the NPC disease is NPC1.
252. The method of 249-251, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
253. The method of 252, wherein the pharmaceutically acceptable salt is citrate.
254. The method of 253, wherein the compound of Formula I is arimoclomol citrate.
255. The method of 254, wherein the compound having structure of formula (I) is N-[(2R,Z)-2-hydroxy-3-(1-piperidyl)propoxy]pyridine-3-carboximidoyl chloride, 1-oxide citrate.
256. The method of 249-255, wherein the therapeutically effective amount of the compound having the structure of Formula I is a daily dosage of about 1 mg/kg/day to about 30 mg/kg/day.
257. The method of 256, wherein the daily dosage ranges from about 0.1 mg to about 400 mg.
258. The method of 257, wherein the daily dosage is administered in about 1-3 unit doses per day.
259. The method of 258, wherein the daily dosage is administered in 3 unit doses per day.
260. The method of 259, wherein the unit dosage is selected from the group consisting of 31 mg, 47 mg, 62 mg, 93 mg, and 124 mg.
261. The method of 249, wherein the composition is combined with soft food prior to administration.
262. The method of 249, wherein the composition is dispersed in water prior to administration.
263. The method of 249, wherein the composition is an oral dosage formulation.
264. The method of 263, wherein the oral dosage formulation is a solid oral dosage formulation.
265. The method of 264, wherein the solid oral dosage is selected from the group consisting of tablets, capsules, caplets, films, and powders.
266. The method of 264, wherein the solid oral dosage form further comprises one or more excipients.
267. The method of 263, wherein the oral dosage formulation is a liquid oral dosage formulation.
268. The method of 267, wherein the liquid oral dosage formulation further comprises a thickening agent.
269. The method of 267, wherein the liquid oral dosage formulation is formulated for administration via a feeding tube or gastric tube.
270. The method of 249, wherein the composition is formulated for parenteral administration.
271. The method according to 249-270, wherein the administration of the compound having a structure of Formula I results in a decrease in at least one scale score selected from SARA GAIT, SARA Finger chase, SARA Nose-finger test, SARA Fast alternating hand movements, 9-HPT, and SARA Speech disturbance.
272. The method according to 249-271, wherein the reduction in NPC disease progression is measured by a decrease in at least one scale score selected from SARA GAIT, SARA Finger chase, SARA Nose-finger test, SARA Fast alternating hand movements, 9-HPT, and SARA Speech disturbance.
273. A method of delaying NPC disease progression in a patient in need thereof, with moderate to severe renal impairment, the method comprising the steps of: determining the Estimated Glomerular Filtration Rate (eGFR) of the patient with a mutant Niemann-Pick type-C Protein 1 (NPC1) with decreased functionality; increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I:

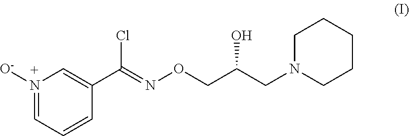

or a pharmaceutically acceptable salt thereof, wherein administering the compound having a structure of Formula I reduces the progression of NPC disease as compared to a placebo group.
274. The method of 273, wherein the Estimated Glomerular Filtration Rate (eGFR) of the subject/patient with moderate to severe renal impairment ranges from 15 mL/min to 50 mL/min.
275. The method of 273 or 274, wherein the NPC disease is selected from the group consisting of NPC, Type 1 (NPC-1) and NPC Type 2 (NPC2).
276. The method of 275, wherein the NPC disease is NPC1.
277. The method of 273-276, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
278. The method of 277, wherein the pharmaceutically acceptable salt is citrate.
279. The method of 278, wherein the compound of Formula I is arimoclomol citrate.
280. The method of 279, wherein the compound having structure of formula (I) is N-[(2R,Z)-2-hydroxy-3-(1-piperidyl)propoxy]pyridine-3-carboximidoyl chloride, 1-oxide citrate.
281. The method of 273-280, wherein the therapeutically effective amount of the compound having the structure of Formula I is a daily dosage of about 1 mg/kg/day to about 15 mg/kg/day.
282. The method of 281, wherein the daily dosage is about 250 mg.
283. The method of 282, wherein the daily dosage is administered in about 1-3 unit doses per day.
284. The method of 283, wherein the daily dosage is administered in 2 unit doses per day.
285. The method of 284, wherein the unit dosage is selected from the group consisting of 31 mg, 47 mg, 62 mg, 93 mg, and 124 mg.
286. The method of 273, wherein the composition is combined with soft food prior to administration.
287. The method of 273, wherein the composition is dispersed in water prior to administration.
288. The method of 273, wherein the composition is an oral dosage formulation.
289. The method of 288, wherein the oral dosage formulation is a solid oral dosage formulation.
290. The method of 289, wherein the solid oral dosage is selected from the group consisting of tablets, capsules, caplets, films, and powders.
291. The method of 289, wherein the solid oral dosage form further comprises one or more excipients.
292. The method of 288, wherein the oral dosage formulation is a liquid oral dosage formulation.
293. The method of 292, wherein the liquid oral dosage formulation further comprises a thickening agent.
294. The method of 292, wherein the liquid oral dosage formulation is formulated for administration via a feeding tube or gastric tube.
295. The method of 273, wherein the composition is formulated for parenteral administration.
296. The method according to 273-295, wherein the administration of the compound having a structure of Formula I results in a decrease in at least one scale score selected from SARA GAIT, SARA Finger chase, SARA Nose-finger test, SARA Fast alternating hand movements, 9-HPT, and SARA Speech disturbance.
297. The method according to 273-296, wherein the delay in NPC disease progression is measured by a decrease in at least one scale score selected from SARA GAIT, SARA Finger chase, SARA Nose-finger test, SARA Fast alternating hand movements, 9-HPT, and SARA Speech disturbance. The presently described technology is now described in such full, clear, concise and exact terms as to enable any person skilled in the art to which it pertains, to practice the same. It is to be understood that the foregoing describes preferred embodiments of the technology and that modifications may be made therein without departing from the spirit or scope of the invention as set forth in the appended claims.

1. A method of increasing removal of lysosomal cholesterol from cells of a patient in need thereof, the method comprising the steps of:
increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I: 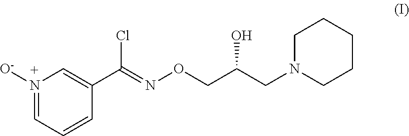
or a pharmaceutically acceptable salt thereof,
where increasing the activation of the at least one transcription factor increases the removal of lysosomal cholesterol from the cells of the patient relative to a patient not administered a therapeutically effective amount of the compound of Formula I, or a pharmaceutically acceptable salt thereof.

2. (canceled)

3. The method of claim 1, wherein the patient has a disease associated with impaired cholesterol trafficking.

4. The method of claim 3 wherein the disease is NPC (Niemann-Pick, Type C disease).

5. The method of claim 4, wherein the NPC disease is selected from the group consisting of NPC, Type 1 (NPC-1) and NPC Type 2 (NPC2).

6. The method of claim 5, wherein the NPC disease is NPC1.

7. The method of claim 1, wherein the pharmaceutically acceptable salt is selected from the group consisting of sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

8. The method of claim 7, wherein the pharmaceutically acceptable salt is citrate.

9. The method of claim 8, wherein the compound of Formula I is arimoclomol citrate.

10. The method of claim 9, wherein the compound having structure of formula (I) is N-[(2R,Z)-2-hydroxy-3-(1-piperidyl)propoxy]pyridine-3-carboximidoyl chloride, 1-oxide citrate.

11. The method of claims 1-10, wherein the therapeutically effective amount of the compound having the structure of Formula I is a daily dosage of about 1 to about 30 mg/kg/day.

12. The method of claim 11, wherein the daily dosage ranges from about 0.1 mg to about 400 mg.

13. The method of claim 12, wherein the daily dosage is administered in about 1-3 unit doses per day.

14. The method of claim 13, wherein the daily dosage is administered in 3 unit doses per day.

15. The method of claim 13, wherein the unit doses are selected from the group consisting of 31 mg, 47 mg, 62 mg, 93 mg, and 124 mg.

16. The method of claim 1, wherein the composition is combined with soft food prior to administration.

17. The method of claim 1, wherein the composition is dispersed in water prior to administration.

18. The method of claim 1, wherein the composition is an oral dosage formulation.

19. The method of claim 18, wherein the oral dosage formulation is a solid oral dosage formulation.

20. The method of claim 19, wherein the solid oral dosage is selected from the group consisting of tablets, capsules, caplets, films, and powders.

21. (canceled)

22. The method of claim 18, wherein the oral dosage formulation is a liquid oral dosage formulation.

23. The method of claim 22, wherein the liquid oral dosage formulation further comprises a thickening agent.

24. The method of claim 22, wherein the liquid oral dosage formulation is formulated for administration via a feeding tube or gastric tube.

25. The method of claim 1, wherein the composition is formulated for parenteral administration.

26.-297. (canceled)

298. A method of increasing removal of endosomal cholesterol from cells of a patient in need thereof, the method comprising:
increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I: 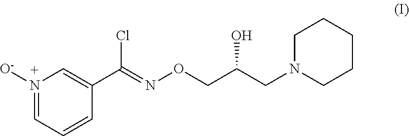
or a pharmaceutically acceptable salt thereof,
where increasing the activation of the at least one transcription factor increases the removal of endosomal cholesterol from the cells of the patient relative to a patient not being administered a therapeutically effective amount of the compound having a structure of Formula I, or a pharmaceutically acceptable salt thereof.

299. A method of increasing cellular production of NPC1 protein in a patient with a mutant Niemann-Pick type-C Protein 1 (NPC1) with decreased functionality compared to wild-type NPC1 protein, the method comprising the steps of. increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I: 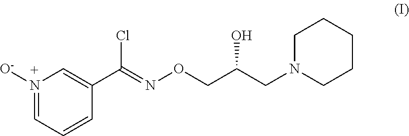
or a pharmaceutically acceptable salt thereof,
where increasing the activation of the at least one transcription factor increases cellular production of the mutant NPC1 protein having reduced functionality compared to wild-type NPC1 protein relative to a patient not being administered a therapeutically effective amount of the compound having a structure of Formula I, or a pharmaceutically acceptable salt thereof.

300. A method for improving at least one 4D-NPCCSS scale domain score in a patient in need thereof, the method comprising the steps of:
increasing activation of at least one transcription factor selected from the group consisting of Transcription Factor EB (TFEB) and Transcription Factor E3 (TFE3), by administering to the patient in need thereof a composition comprising a therapeutically effective amount of a compound having a structure of Formula I: 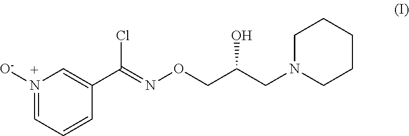
or a pharmaceutically acceptable salt thereof,
where increasing the activation of the at least one transcription factor decreases at least one 4D-NPCCSS domain score, wherein the at least one 4D-NPCCSS domain is selected from ambulation, fine motor skills, and speech relative to a patient not being administered a therapeutically effective amount of the compound having a structure of Formula I, or a pharmaceutically acceptable salt thereof.
